(19) United States
(12) Patent Application Publication
(10) Pub. No.: US 2007/0191371 A1
(43) Pub. Date: Aug. 16, 2007

(54) HETEROCYCLIC MODULATORS OF PPAR

(75) Inventors: Dennis A. Bennett, San Diego, CA (US); Daniel L. Severance, San Diego, CA (US); J. Edward Semple, San Diego, CA (US)

Correspondence Address: GLOBAL PATENT GROUP;ATTN: MS LAVERN HALL, P.O. BOX 38100, ST. LOUIS, MO, 63138, US

(73) Assignee: KALYPSYS, INC., San Diego, CA (US)

(21) Appl. No.: 11/675,067

(22) Filed: Feb. 14, 2007

Related U.S. Application Data
(60) Provisional application No. 60/773,289, filed on Feb. 14, 2006.

(51) Int. Cl. A61K 31/53; A61K 31/501; A61K 31/50; A61K 31/4965; A61K 31/497; A61K 31/4164; A61K 31/4192; A61K 31/4439; A61K 31/4152; A61K 31/381

(52) U.S. Cl. 514/242; 514/341; 514/343; 514/396; 514/359; 514/406; 514/408; 514/372; 514/378; 514/438; 514/252.1; 514/255.05; 514/247; 514/252.03; 514/332; 544/182; 544/224; 544/238; 544/333; 544/405; 544/336; 546/268.4; 546/273.1; 546/275.1; 548/254; 548/344.1; 548/376.1; 548/571; 549/78

(57) ABSTRACT

The present invention relates to compounds and methods useful as modulators of Peroxisome Proliferator-Activated Receptors (PPARs) for treatment or prevention of disease.

This application claims the benefit of priority of U.S. provisional application No. 60/773,289, filed Feb. 14, 2006, the disclosure of which is hereby incorporated by reference as if written herein in its entirety.

### FIELD OF THE INVENTION

The present invention is directed to new heteroaryl and aryl compounds and compositions and their application as pharmaceuticals for the treatment of disease. Methods of modulation of PPAR activity in a human or animal subject are also provided for the treatment diseases such as dyslipidemia, hyperlipidemia, hypercholesteremia, metabolic syndrome X, atherosclerosis, atherogenesis, heart failure, myocardial infarction, vascular diseases, cardiovascular disease, type II diabetes mellitus, type I diabetes, insulin resistance, hypertension, obesity, anorexia bulimia, anorexia nervosa, hair growth abnormalities, skin disorders, inflammation, arthritis, cancer, Alzheimer's disease, respiratory diseases, ophthalmic disorders, IBD (irritable bowel disease), ulcerative colitis and Crohn's disease.

### BACKGROUND OF THE INVENTION

Peroxisome proliferators are a structurally diverse group of compounds which, when administered to mammals, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, Ann. Rev. Cell Biol. 1:489-530 (1985); Vamecq and Draye, Essays Biochem. 24:1115-225 (1989); and Nelali et al., Cancer Res. 48:5316-5324 (1988)). Compounds that activate or otherwise interact with one or more of the PPARs have been implicated in the regulation of triglyceride and cholesterol levels in animal models. Compounds included in this group are the fibrate class of hypolipidemic drugs, herbicides, and phthalate plasticizers (Reddy and Lalwani, Crit. Rev. Toxicol. 12:1-58 (1983)). Peroxisome proliferation can also be elicited by dietary or physiological factors such as a high-fat diet and cold acclimatization.

Biological processes modulated by PPAR are those modulated by receptors, or receptor combinations, which are responsive to the PPAR receptor ligands. These processes include, for example, plasma lipid transport and fatty acid catabolism, regulation of insulin sensitivity and blood glucose levels, which are involved in hypoglycemia/hyperinsulinemia (resulting from, for example, abnormal pancreatic beta cell function, insulin secreting tumors and/or autoimmune hypoglycemia due to autoantibodies to insulin, the insulin receptor, or autoantibodies that are stimulatory to pancreatic beta cells), macrophage differentiation which lead to the formation of atherosclerotic plaques, inflammatory response, carcinogenesis, hyperplasia, and adipocyte differentiation.

Subtypes of PPAR include PPAR-alpha, PPAR-delta (also known as NUC1, PPAR-beta and FAAR) and two isoforms of PPAR-gamma. These PPARs can regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endoodn. Met. 291-296, 4 (1993)).

Insight into the mechanism whereby peroxisome proliferators exert their pleiotropic effects was provided by the identification of a member of the nuclear hormone receptor superfamily activated by these chemicals (Isseman and Green, Nature 347-645-650 (1990)). The receptor, termed PPAR-alpha (or alternatively, PPARα), was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid ω-hydroxylase (Gottlicher et al., Proc. Natl. Acad. Sci. USA 89:4653-4657 (1992); Tugwood et al., EMBO J 11:433-439 (1992); Bardot et al., Biochem. Biophys. Res. Comm. 192:37-45 (1993); Muerhoff et al., J Biol. Chem. 267:19051-19053 (1992); and Marcus et al., Proc. Natl. Acad. Sci. USA 90(12):5723-5727 (1993).

Activators of the nuclear receptor PPAR-gamma (or alternatively, PPARγ), for example troglitazone, have been clinically shown to enhance insulin-action, to reduce serum glucose and to have small but significant effects on reducing serum triglyceride levels in patients with Type 2 diabetes. See, for example, D. E. Kelly et al., Curr. Opin. Endocrinol. Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997).

PPAR-delta (or alternatively, PPARδ) initially received much less attention than the other PPARs because of its ubiquitous expression and the unavailability of selective ligands. However, genetic studies and recently developed synthetic PPAR-δ agonists have helped reveal its role as a powerful regulator of fatty acid catabolism and energy homeostasis. Studies in adipose tissue and muscle have begun to uncover the metabolic functions of PPAR-δ. Transgenic expression of an activated form of PPAR-δ in adipose tissue produces lean mice that are resistant to obesity, hyperlipidemia and tissue steatosis induced genetically or by a high-fat diet. The activated receptor induces genes required for fatty acid catabolism and adaptive thermogenesis. Interestingly, the transcription of PPAR-δ target genes for lipid storage and lipogenesis remain unchanged. In parallel, PPAR-δ-deficient mice challenged with a high-fat diet show reduced energy uncoupling and are prone to obesity. Together, these data identify PPAR-δ as a key regulator of fat-burning, a role that opposes the fat-storing function of PPAR-γ. Thus, despite their close evolutionary and structural kinship, PPAR-y and PPAR-δ regulate distinct genetic networks. In skeletal muscle, PPAR-δ likewise upregulates fatty oxidation and energy expenditure, to a far greater extent than does the lesser-expressed PPAR-α. (Evans R M et al 2004 Nature Med 1-7, 10 (4), 2004)

PPAR-δ is broadly expressed in the body and has been shown to be a valuable molecular target for treatment of dyslipidemia and other diseases. For example, in a recent study in insulin-resistant obese rhesus monkeys, a potent and selective PPAR-delta compound was shown to decrease VLDL and increase HDL in a dose response manner (Oliver et al., Proc. Natl. Acad. Sci. U.S.A. 98: 5305, 2001).

Because there are three isoforms of PPAR and all of them have been shown to play important roles in energy homeostasis and other important biological processes in human body and have been shown to be important molecular targets for treatment of metabolic and other diseases (see Willson, et al. J. Med. Chem. 43: 527-550 (2000)), it is desired in the art to identify compounds which are capable of interacting with multiple PPAR isoforms or compounds which are capable of selectively interacting with only one of the PPAR isoforms, preferably PPARδ. Such compounds would find a wide variety of uses, such as, for example, in the treatment or prevention of obesity, for the treatment or prevention of diabetes, dyslipidemia, metabolic syndrome X and other uses.

Several PPAR-modulating drugs have been approved for use in humans. Fenofibrate and gemfibrozil are PPARγ modulators; pioglitazone (Actos, Takeda Pharmaceuticals and Eli Lilly) and rosiglitazone (Avandia, GlaxcoSmithKline) are PPARα modulators. All of these compounds have liabilitits as potential carcinogens, however, having been demonstrated to have proliferative effects leading to cancers of various types (colon; bladder with PPARα modulators and liver with PPARγ modulators) in rodent studies. Therefore, a need exists to identify modulators of PPARs that lack these liabilities.

### SUMMARY OF THE INVENTION

Novel compounds and pharmaceutical compositions that modulate PPAR have been found, together with methods of synthesizing and using the compounds including methods for modulating PPAR in a patient by administering the compounds.

The present invention discloses a class of compounds, useful in treating PPAR-mediated disorders and conditions, defined by structural Formula I: 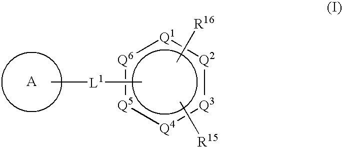
or a salt, ester, or prodrug thereof, wherein:

A is selected from the group consisting of aryl, heteroaryl, cycloalkyl, and heterocycloalkyl, any of which may be optionally substituted;

L¹ is selected from the group consisting of —X—, —XOX—, —XS(O)₀₋₂X— and —XS(O)₀₋₂XO—, —XOXOX— —XN(R¹)X—, —XS(O)₂N(R¹)X—, —XC(O)N(R¹)X—, —X(CF₂)₁₋₃X—, —XC(═O)X—, —XC(═O)OX—, —XN(R¹)C(═O)X—, —XN(R¹)C(═O)N(R²)X—, —XN(R¹)SO₂N(R²)X—, —XN(R¹)C(═O)OX, —XP(═O)(OR¹)X—, —XP(═O)(NR¹)X—, —XP(═S)(OR¹)X—, —XP(═S)(NR¹)X—, and —XS(═O)(═NR¹)X—;

R¹ and R² are hydrogen or are independently selected from the group consisting of lower alkyl, lower alkoxy, lower perhaloalkyl, lower alkenyl, lower alkynyl, lower heteroalkyl, lower alkoxyalkyl, alkylamino, alkylaminoalkyl, alkylcarbonyl, amido, aminoalkyl, aryl, arylalkyl, arylalkenyl, arylalkynyl, lower cycloalkyl, lower cycloalkyl alkyl, lower haloalkyl, heteroaryl, heteroarylalkyl, heteroarylalkenyl, and heterocycloalkyl, any of which may be optionally substituted; or R¹ and R², together with the atoms to which they are attached, may be joined to form an optionally substituted heterocycloalkyl or optionally substituted cycloalkyl moiety;

X is a bond, or is selected from lower alkyl and lower alkenyl, which may be optionally substituted;

Q¹-Q⁶ are independently selected from the group consisting of a bond, C, NR³, S, and O;

R³ is hydrogen or is selected from the group consisting of lower alkyl, lower alkenyl, aryl, arylalkyl, lower cycloalkyl, lower cycloalkyl alkyl, lower haloalkyl, heteroaryl, heteroarylalkyl, heterocycloalkyl, R¹⁵ and R¹⁶, any of which may be optionally substituted;

R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted;

Y is selected from the group consisting of lower alkyl, lower alkenyl, lower alkynyl, —C(O)N(R¹)— and —OX—, any of which may be optionally substituted;

R¹⁸ is selected from the group consisting of —XOXOR⁵, —XOR⁵, —XC(O)OR⁵, —XC(O)NR⁴R⁴, —XC(O)NR⁴OR⁴, —XC(O)OXOR⁵, —XC(O)XOR⁵, —XC(O)N(R⁴)XOR⁵, —XC(O)N(R⁴)(R⁵) and —XC(O)N(R⁴)XR⁵; or R¹⁸ may be further selected from the group consisting of lower cycloalkyl, heterocycloalkyl, aryl and heteroaryl, any of which may be further optionally substituted with —XS(O)₀₋₂R¹, —XS(O)₀₋₂XR¹⁹, —XN(R¹)(R²), —XN(R¹)S(O)₀₋₂R¹, —XN(R¹)C(O)R², —XC(O)N(R¹)(R²), —XN(R¹)(R¹⁹), —XC(O)N(R¹)(R¹⁹), —XC(O)R¹⁹, —XC(O)OR¹, —XC(O)OR¹, —XN(R¹)XR¹⁹ and —XOXR;

R⁴ and R⁵ are hydrogen or are independently selected from the group consisting of lower alkyl, lower cycloalkyl, heterocycloalkyl, aryl and heteroaryl, any of which may be optionally substituted; or R⁴ and R⁵, together with the atom to which they are attached, may be joined to form an optionally substituted heterocycloalkyl or optionally substituted heteroaryl moiety; and

R¹⁹ is selected from the group consisting of lower cycloalkyl, heterocycloalkyl, aryl and heteroaryl, any of which may be optionally substituted.

The present invention further provides compounds of the Formula I,

wherein the cyclic moiety represented by the group: 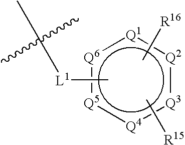
is selected from the heteroaromatic and aromatic groups consisting of:

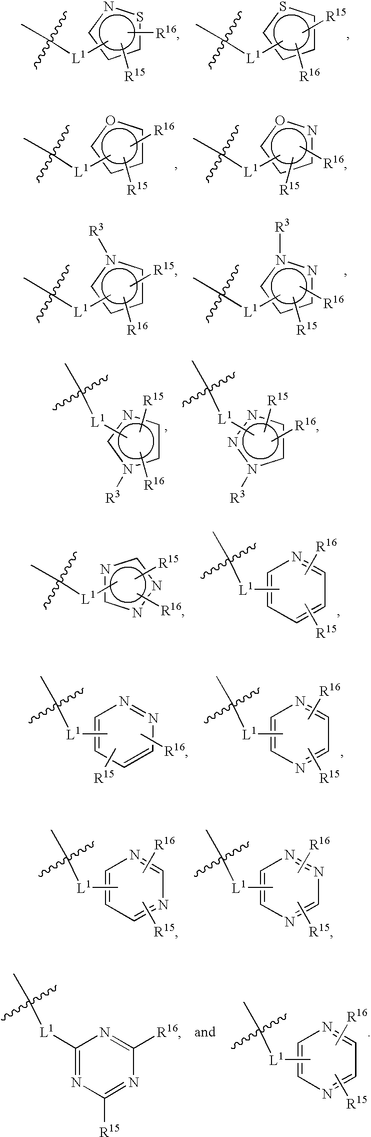

The present invention further provides compounds of the Formula I wherein:

A has a structural formula selected from the group consisting of

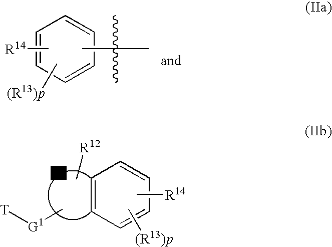

B is an optionally substituted five- to eight-membered carbocycle or heterocyle;

T is selected from the group consisting of hydrogen, —C(O)OH, —C(O)OR¹, —C(O)NH₂, —C(O)N(R¹)(R²), tetrazoly-, and thiazolidine-2,4-dione-5-yl-;

G¹ is selected from the group consisting of —(CR¹R²)ₙ—, -Z(CR¹R²)ₙ—, —(CR¹R²)ₙZ-, and —(CR¹R²)ᵣZ(CR¹R²)ₛ—,

Z is O, S or NR³;

n is an integer from 0 to 2;

r and s are independently integers from 0 to 1;

R¹² is selected from the group consisting of hydrogen, halogen, hydroxy, optionally substituted lower alkyl, optionally substituted lower cycloalkyl, optionally substituted heteroalkyl, optionally substituted cycloheteroalkyl, optionally substituted lower alkynyl, lower perhaloalkyl, lower perhaloalkoxy, optionally substituted lower alkoxy, nitro, cyano, and NH₂;

R¹³ is selected from hydrogen and the group consisting of halogen, lower alkyl lower alkoxy, lower haloalkyl, lower haloalkoxy, aryl, heteroaryl, lower cycloalkyl and heterocycloalkyl, any of which may be optionally substituted;

p is an integer from 0 to 3; and

R¹⁴ is selected from the group consisting of hydrogen, —XOXC(O)OR¹, —XC(O)OH, —XC(O)OR¹, —XC(O)NH₂, —C(O)N(R¹)(R²), —X-tetrazole, and —X-[(thiazolidine-2,4-dione)-5-yl]-.

The present invention also provides pharmaceutical compositions comprising one or more compounds of the present invention together with a pharmaceutically acceptable carrier. In another aspect, the present invention also provides methods for modulating PPAR activity. In a broad aspect, the present invention also provides methods for treating a PPAR-mediated disorder in a human or animal subject in need of such treatment comprising administering to said subject an amount of a compound of the present invention effective to reduce or prevent said disorder in said subject.

In a broad aspect, the subject invention provides for novel compounds, pharmaceutical compositions and methods of making and using the compounds and compositions. These compounds possess useful PPAR modulating activity, and may be used in the treatment or prophylaxis of a disease or condition in which PPAR plays an active role.

### DETAILED DESCRIPTION OF THE INVENTION

In certain embodiments, the compounds of the present invention have structural Formula III:

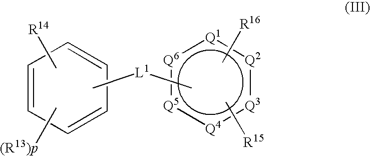

wherein:

L¹ is selected from the group consisting of —X—, —XOX—, —XS(O)₀₋₂X— and —XS(O)₀₋₂XO—, —XOXOX—, —XS(O)₂N(R¹)X—, —XC(O)N(R¹)X—, —X(CF₂)₁₋₃X—, —XN(R¹)C(═O)X—, —XN(R¹)C(═O)N(R²)X—, —XN(R¹)SO₂N(R²)X—, and —XN(R¹)C(═O)OX—;

R¹ and R² are hydrogen or are independently selected from the group consisting of lower alkyl, lower alkoxy, lower alkenyl, lower alkynyl, lower heteroalkyl, lower alkoxyalkyl, alkylaminoalkyl, alkylcarbonyl, aryl, arylalkyl, lower cycloalkyl, lower cycloalkyl alkyl, lower haloalkyl, heteroaryl, heteroarylalkyl, and heterocycloalkyl, any of which may be optionally substituted; or R¹ and R², together with the atoms to which they are attached, may be joined to form an optionally substituted heterocycloalkyl or optionally substituted cycloalkyl moiety;

X is a bond, or is selected from lower alkyl and lower alkenyl, which may be optionally substituted;

Q¹-Q⁶ are independently selected from the group consisting of a bond, C, NR³, S, and O;

R¹³ is selected from hydrogen and from the group consisting of halogen, lower alkyl, lower alkoxy, lower haloalkyl, lower haloalkoxy, lower cycloalkyl and heterocycloalkyl, any of which may be optionally substituted;

p is an integer from 0 to 3;

R¹⁴ is selected from the group consisting of hydrogen, —XOXC(O)OR¹, —XC(O)OH, —XC(O)OR¹, —X-tetrazole, and —X-[(thiazolidine-2,4-dione)-5-yl]-;

R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted;

Y is selected from the group consisting of lower alkyl, lower alkenyl, lower alkynyl, —C(O)N(R¹)— and —OX—, any of which may be optionally substituted;

R¹⁸ is selected from the group consisting of —XOXOR⁵, —XOR⁵, —XC(O)OR⁵, —XC(O)NR⁴R⁴, —XC(O)NR⁴OR⁴, —XC(O)OXOR⁵, —XC(O)XOR⁵, —XC(O)N(R⁴)XOR⁵, —XC(O)N(R⁴)(R⁵) and —XC(O)N(R⁴)XR⁵; or R¹⁸ may be further selected from the group consisting of lower cycloalkyl, heterocycloalkyl, aryl and heteroaryl, any of which may be further optionally substituted with —XS(O)₀₋₂R¹, —XS(O)₀₋₂XR¹⁹, —XN(R¹)(R²), —XN(R¹)S(O)₀₋₂R¹, —XN(R¹)C(O)R², —XC(O)N(R¹)(R²), —XN(R¹)(R¹⁹), —XC(O)N(R¹)(R¹⁹), —XC(O)R¹⁹, —XC(O)OR¹, —XN(R¹)XR¹⁹ and —XOXR;

R⁴ and R⁵ are selected from hydrogen or are independently selected from a group consisting of lower alkyl, lower cycloalkyl, heterocycloalkyl, aryl and heteroaryl, any of which may be optionally substituted; or R⁴ and R⁵, together with the atom to which they are attached, may be joined to form an optionally substituted heterocycloalkyl or optionally substituted heteroaryl moiety; and

R¹⁹ is selected from the group consisting of lower cycloalkyl, heterocycloalkyl, aryl and heteroaryl, any of which may be optionally substituted.

In other embodiments, the compounds of the present invention have structural Formula IV:

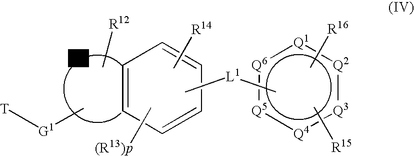

wherein:

B is an optionally substituted five- to eight-membered carbocycle or heterocycle;

T is selected from the group consisting of hydrogen, —C(O)OH, —C(O)OR¹, —C(O)NH₂, —C(O)N(R¹)(R²), tetrazolyl-, and thiazolidine-2,4-dione-5-yl-;

G¹ is selected from the group consisting of —(CR¹R²)ₙ—, -Z(CR¹R²)ₙ—, —(CR¹R²)ₙZ-, and —(CR¹R²)ᵣZ(CR¹R²)ₛ—;

R¹ and R² are hydrogen or are independently selected from the group consisting of lower alkyl, lower alkoxy, lower alkenyl, lower alkynyl, lower heteroalkyl, lower alkoxyalkyl, alkylaminoalkyl, alkylcarbonyl, aryl, arylalkyl, lower cycloalkyl, lower cycloalkyl alkyl, lower haloalkyl, heteroaryl, heteroarylalkyl, and heterocycloalkyl, any of which may be optionally substituted; or R¹ and R², together with the atoms to which they are attached, may be joined to form an optionally substituted heterocycloalkyl or optionally substituted cycloalkyl moiety;

n is an integer from 0 to 2;

r and s are independently integers from 0 to 1;

Z is O, S or NR³;

R³ is hydrogen or is selected from the group consisting of lower alkyl lower alkenyl, aryl, arylalkyl, lower cycloalkyl, lower cycloalkyl alkyl, lower haloalkyl, heteroaryl, heteroarylalkyl, heterocycloalkyl, R¹⁵ and R¹⁶, any of which may be optionally substituted;

L¹ is selected from the group consisting of —X—, —XOX—, —XS(O)₀₋₂X—, —XS(O)₀₋₂XO—, —XOXO—, —XS(O)₂N(R¹)X—, —XC(O)N(R¹)X—, —X(CF₂)₁₋₃X—, —XN(R¹)C(═O)X—, —XN(R¹)C(═O)N(R²)X—, —XN(R¹)SO₂N(R²)X—, and —XN(R¹)C(═O)OX—,

X is a bond, or is selected from lower alkyl and lower alkenyl, which may be optionally substituted;

Q¹-Q⁶ are independently selected from the group consisting of a bond (direct link), C, NR³, S, and O;

R¹² is selected from the group consisting of hydrogen, halogen, optionally substituted lower alkyl optionally substituted lower cycloalkyl, optionally substituted cycloheteroalkyl, lower perhaloalkyl, lower perhaloalkoxy, optionally substituted lower alkoxy, nitro, and cyano;

R¹³ is selected from hydrogen and the group consisting of halogen, lower alkyl, lower alkoxy, lower haloalkyl, lower haloalkoxy, lower cycloalkyl and heterocycloalkyl, any of which may be optionally substituted;

p is an integer from 0 to 3;

R¹⁴ is selected from the group consisting of hydrogen, —XOXC(O)OR¹, —XC(O)OH, —XC(O)OR¹, —X-tetrazole, and —X-[(thiazolidine-2,4-dione)-5-yl]-;

R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted;

Y is selected from the group consisting of lower alkyl, lower alkenyl, lower alkynyl, —C(O)N(R¹)— and —OX—, any of which may be optionally substituted;

R¹⁸ is selected from the group consisting of —XOXOR⁵, —XOR⁵, —XC(O)OR⁵, —XC(O)NR⁴R⁴, —XC(O)NR⁴OR⁴, —XC(O)OXOR⁵, —XC(O)XOR⁵, —XC(O)N(R⁴)XOR⁵, —XC(O)N(R⁴)(R⁵) and —XC(O)N(R⁴)XR⁵; or R¹⁸ may be further selected from the group consisting of lower cycloalkyl, heterocycloalkyl, aryl and heteroaryl, any of which may be further optionally substituted with —XS(O)₀₋₂R¹, —XS(O)₀₋₂XR¹⁹, —XN(R¹)(R²), —XN(R¹)S(O)₀₋₂R¹, —XN(R¹)C(O)R², —XC(O)N(R¹)(R²), —XN(R¹)(R¹⁹), —XC(O)N(R¹)(R¹⁹), —XC(O)R¹⁹, —XC(O)OR¹, —XN(R¹)XR¹⁹ and —XOXR;

R⁴ and R⁵ are independently selected from hydrogen or are selected from a group consisting of lower alkyl, lower cycloalkyl, heterocycloalkyl, aryl and heteroaryl, any of which may be optionally substituted; or R⁴ and R⁵, together with the atom to which they are attached, may be joined to form an optionally substituted heterocycloalkyl or optionally substituted heteroaryl moiety; and

R¹⁹ is selected from the group consisting of lower cycloalkyl, heterocycloalkyl, aryl and heteroaryl, any of which may be optionally substituted.

In other embodiments, the compounds of the present invention have the structural Formula III wherein:

L¹ is selected from the group consisting of —X—, —XOX—, —XS(O)₀₋₂X—, —XOXO—, and —XS(O)₀₋₂XO—;

X is a bond or optionally substituted alkyl;

R¹³ is selected from the group consisting of hydrogen, optionally substituted lower alkyl, optionally substituted lower alkoxy and halogen;

R¹⁴ is selected from the group consisting of hydrogen, —XOXC(O)OR¹ and —XC(O)OR¹;

R¹ is selected from hydrogen and optionally substituted lower alkyl; and

R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted.

In other embodiments, the compounds of the present invention have the structural Formula IIIa:

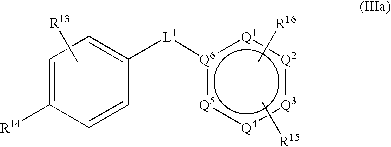

wherein:

L¹ is selected from the group consisting of —S(O)₀₋₂(CH₂)₁₋₄O—, —O(CH₂)₁₋₄S(O)₀₋₂—CH₂S(O)₀₋₂—, —S(O)₀₋₂CH₂—, —S(O)₀₋₂—, —OCH₂CH₂O—, —CH₂O— and —OCH₂—;

R¹³ is selected from is selected from the group consisting of hydrogen, optionally substituted lower alkyl, optionally substituted lower alkoxy and halogen;

R¹⁴ is selected from is selected from the group consisting of hydrogen, —OCH₂C(O)OH, —CH₂C(O)OH, —OC(CH₃)₂C(O)OH, —CH═CH—C(O)OH, —(CH₂)₂C(O)OH, and —OCH₂-tetrazolyl;

R¹⁵ and R¹⁶ are independently selected from —R¹⁸ and —YR¹⁸; or R¹⁵ and R¹⁶ together with the atoms to which R¹⁵ and R¹⁶ are attached form 4,5-dihydro-naphtho[1,2-d]naphthal-2-yl, 4H-chromeno[4,3-d]naphthal-2-yl, 5,6-dihydro-4H-3-thia-1-aza-benzo[e]azulen-2-yl, benzthiazolyl, benzoxazolyl and 1-oxa-3-aza-cyclopenta[a]naphthalene-2-yl, any of which may be optionally substituted;

Y is selected from the group consisting of optionally substituted lower alkyl, optionally substituted lower alkenyl, —C(O)NH— and —O(CH₂)₁₋₃—; and

R¹⁸ is selected from the group consisting of —XOR⁴, —XC(O)OR⁴, —XC(O)OXOR⁴, —XC(O)N(R⁴)XOR⁵, —XC(O)N(R⁴)(R⁵), —XC(O)N(R⁴)XR⁵, phenyl, biphenyl, cyclohexyl, naphthyl, benzo[1,3]dioxol-5-yl, benzo[b]furanyl, pyridinyl, pyrimidinyl, dibenzo-furan-2-yl, furanyl, benzo[b]thiophene, thiophenyl, phenoxathiin-4-yl, benzoxazolyl, 3-oxo-3,4-dihydro-2H-benzo[1,4]oxazin-6-yl, 2-oxo-2,3-dihydro-benzooxazol-6-yl, 2,3-dihydro-benzo[1,4]dioxin-6-yl, benzoxazolyl, 3,4-dihydro-2H-benzo[b][1,4]-dioxepin-7-yl and quinolinyl; any of which may be optionally substituted.

In certain embodiments, the compounds of the present invention have structural Formula Ia selected from the group consisting of:

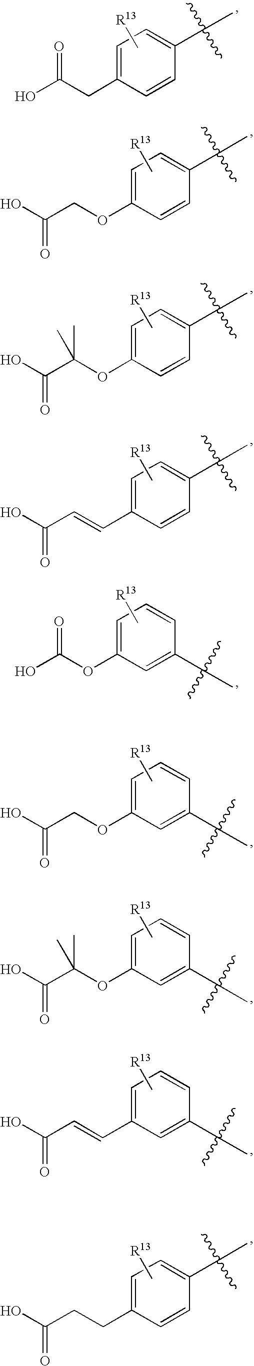

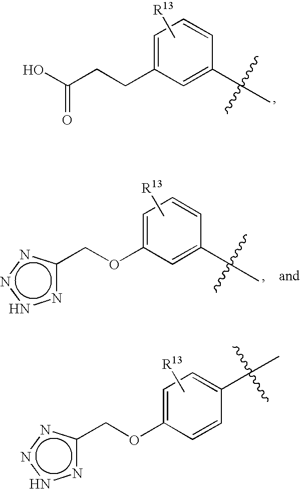

wherein:

R¹³ is selected from the group consisting of hydrogen, halogen, lower alkyl and lower alkoxy.

In certain embodiments, the compounds of the present invention have structural Formula V:

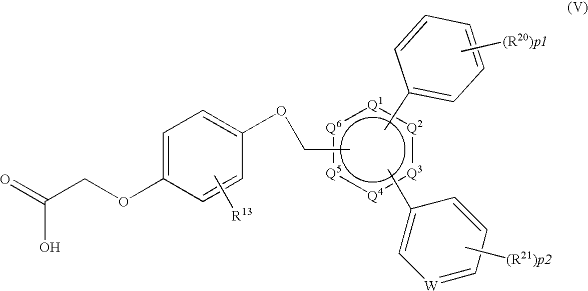

wherein:

W is selected from N and CH;

R¹³ is selected from the group consisting of hydrogen, optionally substituted lower alkyl optionally substituted lower alkoxy, and halogen;

R²⁰ is selected from trifluoromethyl and trifluoromethoxy;

R²¹ is selected from isopropyloxy and methoxy; and

p1 and p2 are independently selected from an integer from 0 to 2.

In preferred embodiments, the Formula V compounds of the present invention have the structural formula selected from the group consisting of:

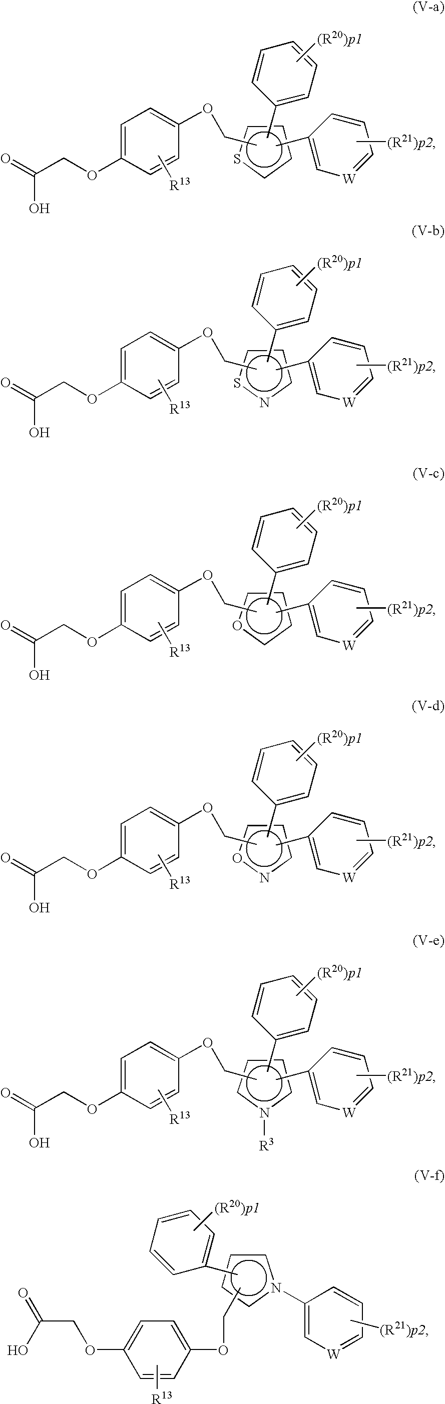

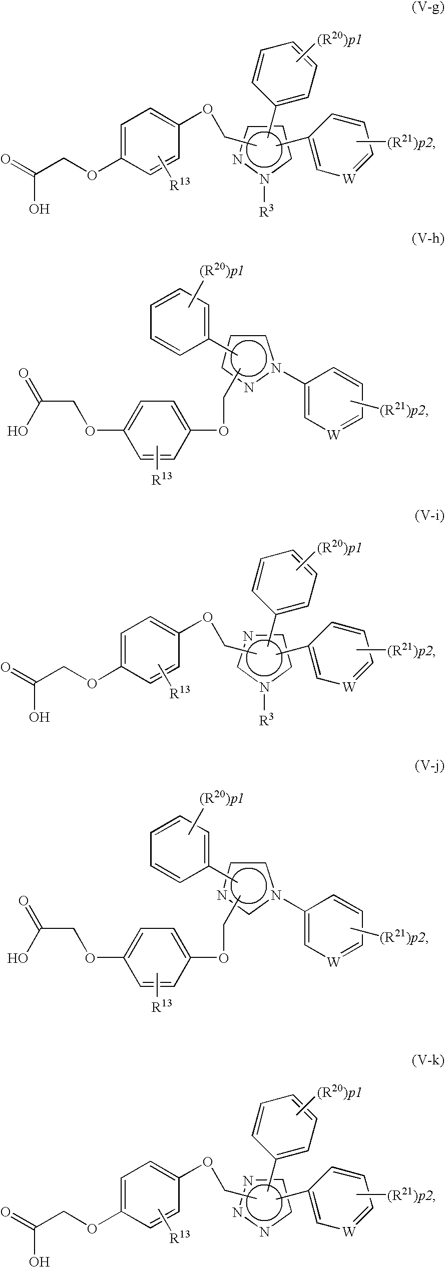

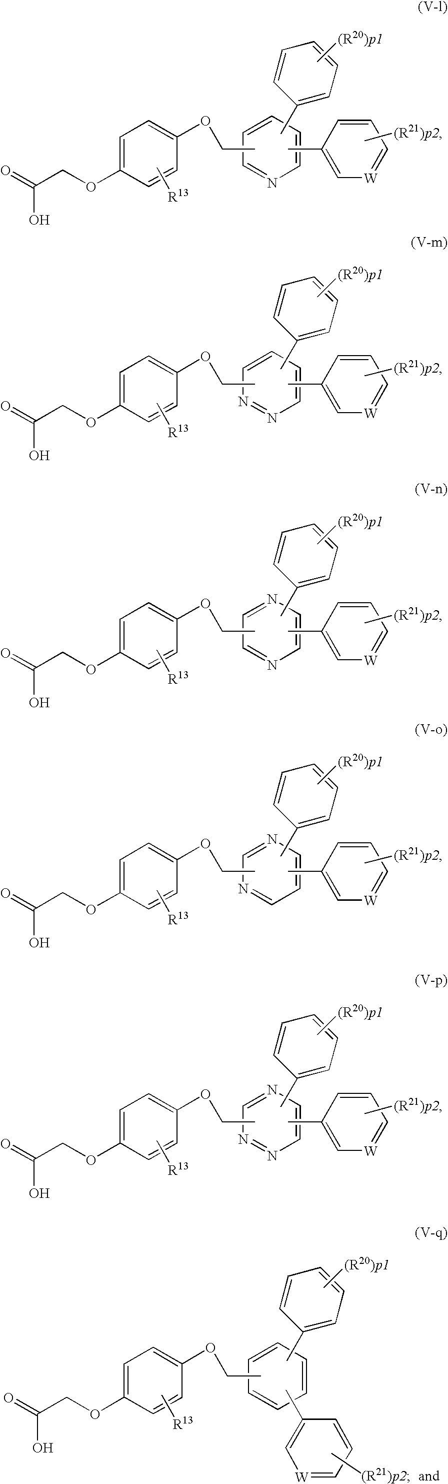

wherein the (R²⁰)ₚ₁-aryl and (R²¹)ₚ₂-(hetero)aryl moieties are attached to two contiguous central ring atoms.

In certain embodiments, the compounds of the present invention have structural Formula VI:

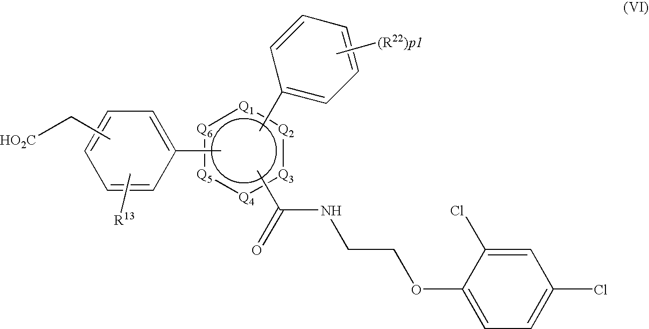

wherein:

R¹³ is selected from the group consisting of hydrogen, optionally substituted lower alkyl optionally substituted lower alkoxy and halogen;

R²² is selected from the group consisting of hydrogen, methoxy, isopropyloxy, trifluoromethoxy, fluoro, chloro, trifluoromethyl, methyl, and nitro; and

p1 is an integer from 0 to 2.

In preferred embodiments, the compounds of the present invention have structural Formula VI, selected from the group consisting of:

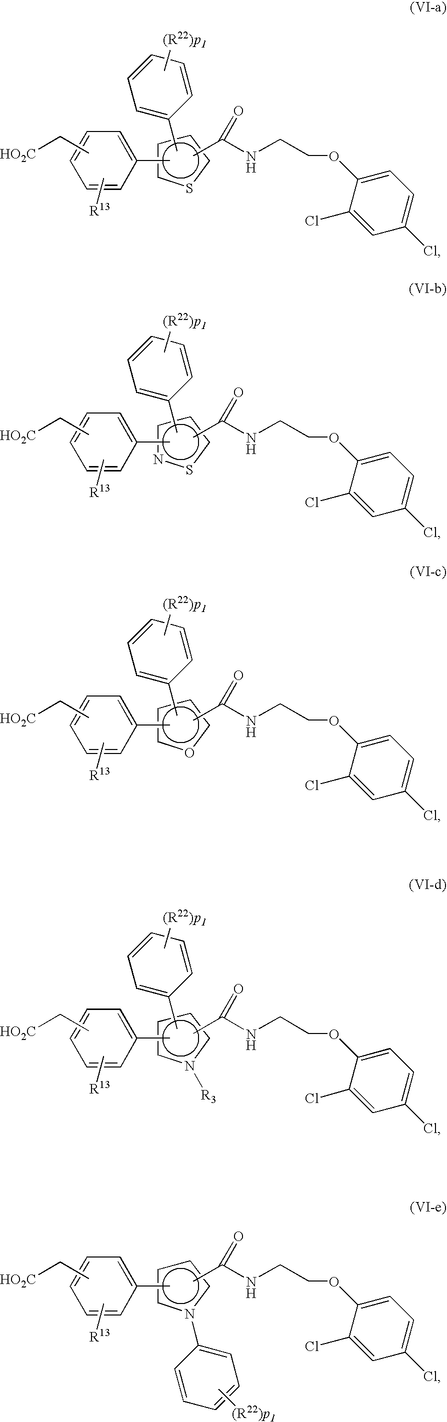

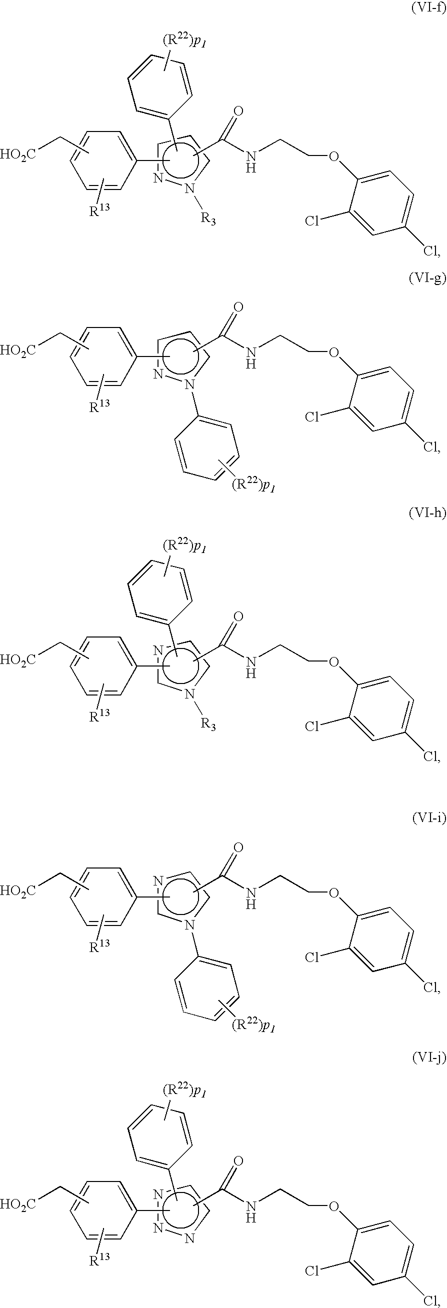

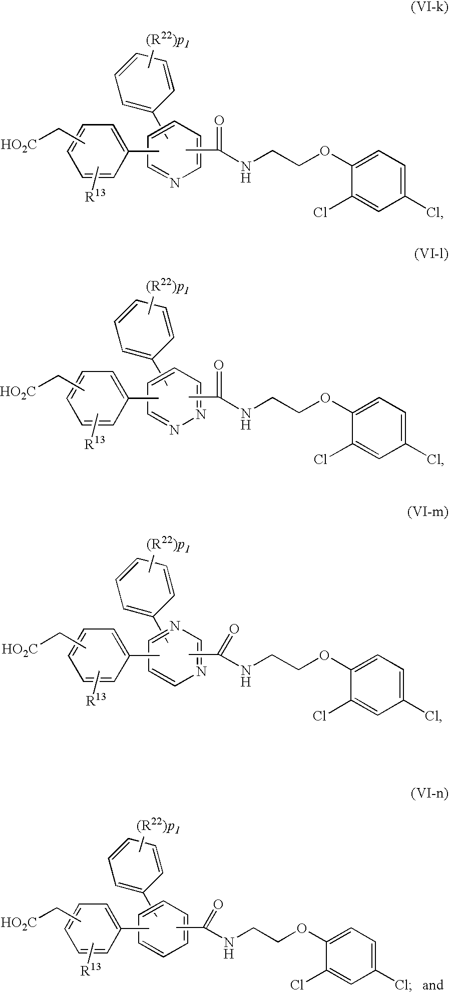

wherein the three substituents are attached to three contiguous ring atoms of the central ring moiety.

In certain embodiments, the compounds of the present invention have structural Formula IV wherein:

T is selected from the group consisting of —C(O)OH, —C(O)OR¹, and tetrazolyl;

G¹ is selected from the group consisting of —(CR¹R²)ₙ—, —(CR¹R²)ₙZ-, and —(CR¹R²)ᵣZ(CR¹R²)ₛ—;

R¹ and R² are hydrogen or optionally substituted lower alkyl;

Z is O, S or NR³;

R³ is hydrogen or is selected from the group consisting of lower alkyl, R¹⁵, and R¹⁶, any of which may be optionally substituted;

L¹ is selected from the group consisting of —X—, —XOX—, —XS(O)₀₋₂X—, —XOXO—, and —XS(O)₀₋₂XO—;

R¹² is selected from the group consisting of hydrogen, halogen, optionally substituted lower alkyl, lower perhaloalkyl, lower perhaloalkoxy, optionally substituted lower alkoxy, nitro, and cyano;

R¹³ is selected from the group consisting of hydrogen, optionally substituted lower alkyl, optionally substituted lower alkoxy and halogen;

R¹⁴ is selected from the group consisting of hydrogen, —XOXC(O)OR¹ and —XC(O)OR¹; and

R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted.

In certain embodiments, the compounds of the present invention have structural Formula IV wherein:

T is —C(O)OH, and tetrazolyl;

G¹ is —(CR¹R²)ₙ;

L¹ is selected from the group consisting of a bond, —S(O)₀₋₂(CH₂)₁₋₄O—, —O(CH₂)₁₋₄S(O)₀₋₂—, —CH₂S(O)₀₋₂—, —O(CH₂)₁₋₄O—, —S(O)₀₋₂CH₂—, —S(O)₀₋₂—, —CH₂O— and —OCH₂—;

R¹⁴ is hydrogen;

R¹⁵ and R¹⁶ are independently selected from —R¹⁸ and —YR¹⁸; or R¹⁵ and R¹⁶ together with the atoms to which R¹⁵ and R¹⁶ are attached form 4,5-dihydro-naphtho[1,2-d]naphthal-2-yl, 4H-chromeno[4,3-d]naphthal-2-yl, 5,6-dihydro-4H-3-thia-1-aza-benzo[e]azulen-2-yl, benzthiazolyl, benzoxazolyl and 1-oxa-3-aza-cyclopenta[a]naphthalene-2-yl, any of which may be optionally substituted;

Y is selected from the group consisting of optionally substituted lower alkyl, optionally substituted lower alkenyl, —C(O)NH— and —O(CH₂)₁₋₃—; and

R¹⁸ is selected from the group consisting of —XOR⁴, —XC(O)OR⁴, —XC(O)OXOR⁴, —XC(O)N(R⁴)XOR⁵, —XC(O)N(R⁴)(R⁵), —XC(O)N(R⁴)XR⁵, phenyl, biphenyl, cyclohexyl, naphthyl, benzo[1,3]dioxol-5-yl, benzo[b]furanyl, pyridinyl, pyrimidinyl, dibenzo-furan-2-yl, furanyl, benzo[b]thiophene, thiophenyl, phenoxathiin-4-yl, benzoxazolyl, 3-oxo-3,4-dihydro-2H-benzo[1,4]oxazin-6-yl, 2-oxo-2,3-dihydro-benzooxazol-6-yl, 2,3-dihydro-benzo[1,4]dioxin-6-yl, benzoxazolyl, 3,4-dihydro-2H-benzo[b][1,4]-dioxepin-7-yl and quinolinyl; any of which may be optionally substituted.

In certain embodiments, the compounds of the present invention have structural Formula (IIb) selected from the group consisting of:

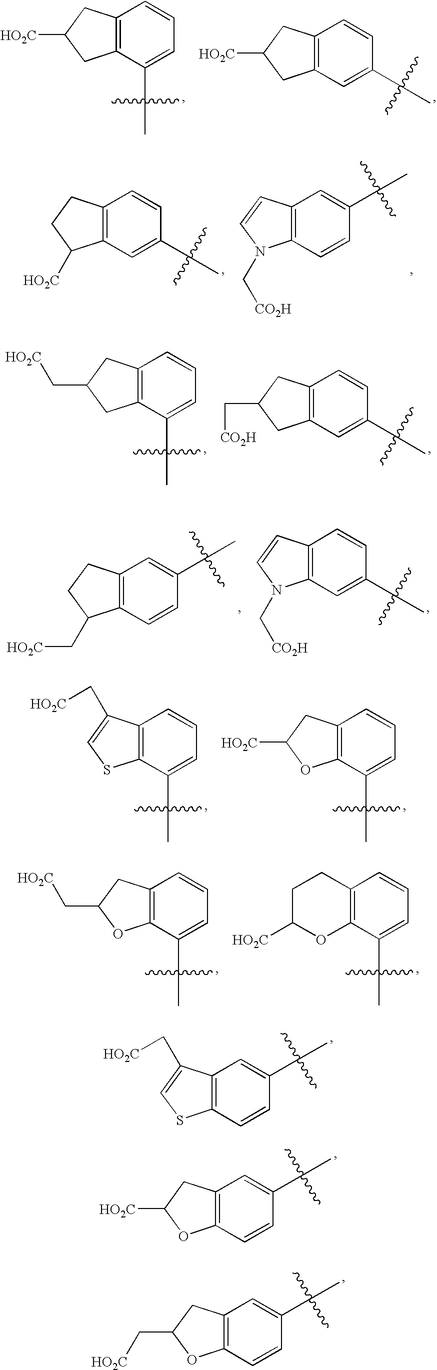

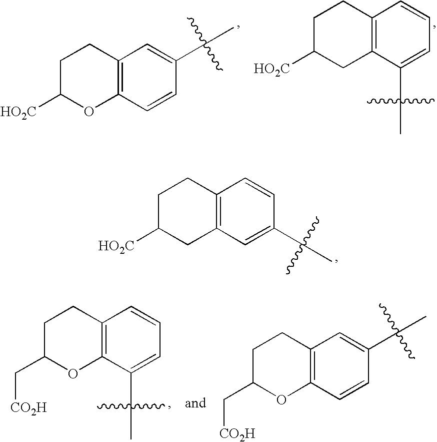

In preferred embodiments, the compounds of the present invention have structural Formula VII:

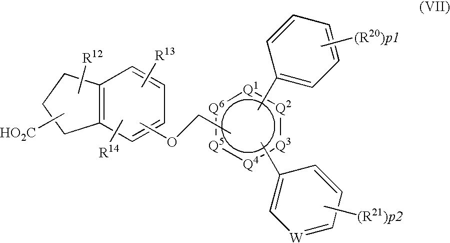

wherein:

W is selected from N and CH;

R¹², R¹³ and R¹⁴ are independently selected from the group consisting of hydrogen, optionally substituted lower alkyl optionally substituted lower alkoxy and halogen;

R²⁰ is selected from trifluoromethyl and trifluoromethoxy;

R²¹ is selected from isopropyloxy and methoxy; and

p1 and p2 are independently selected from an integer from 0 to 2.

In preferred embodiments, the compounds of the present invention have structural Formula VII selected from the group consisting of:

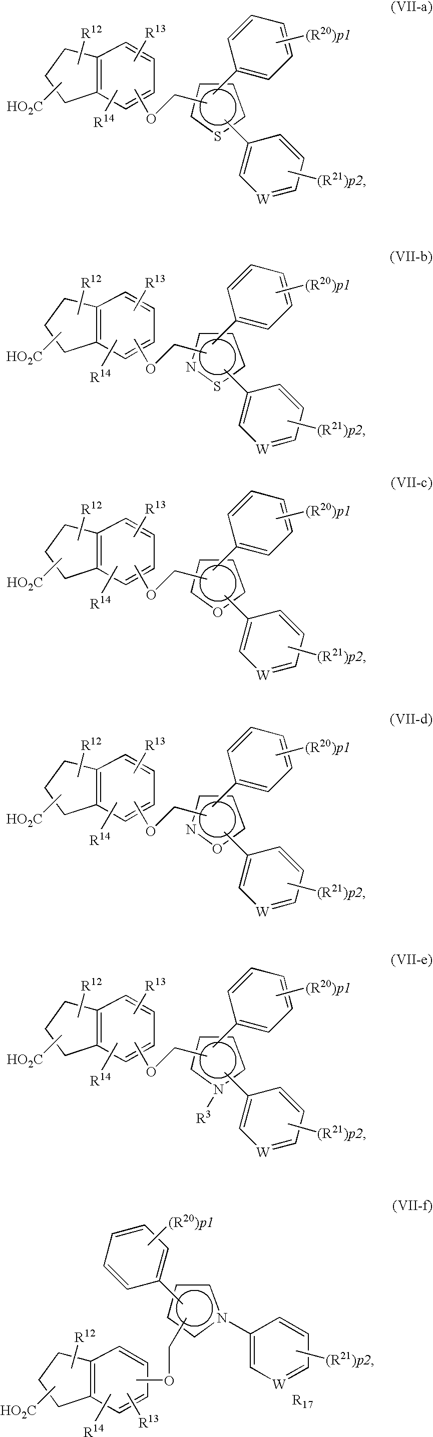

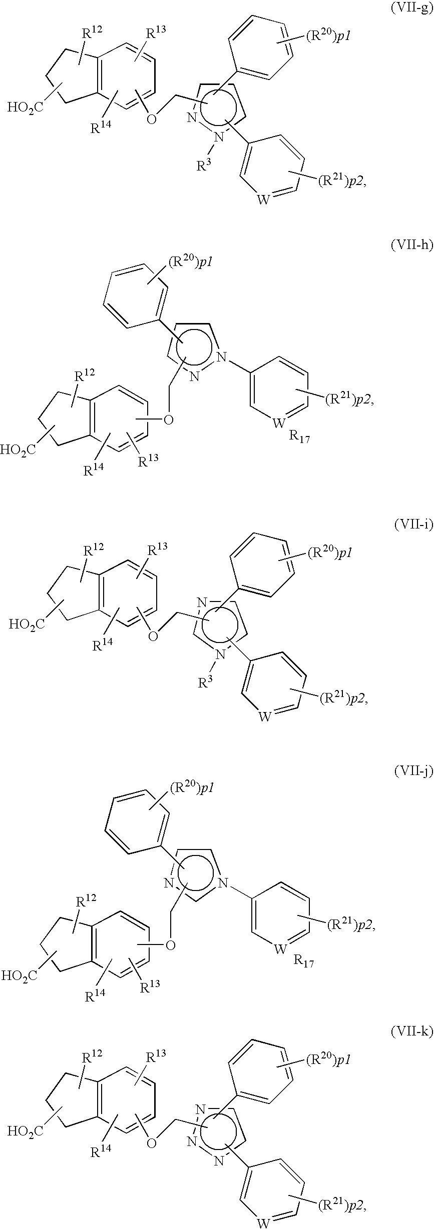

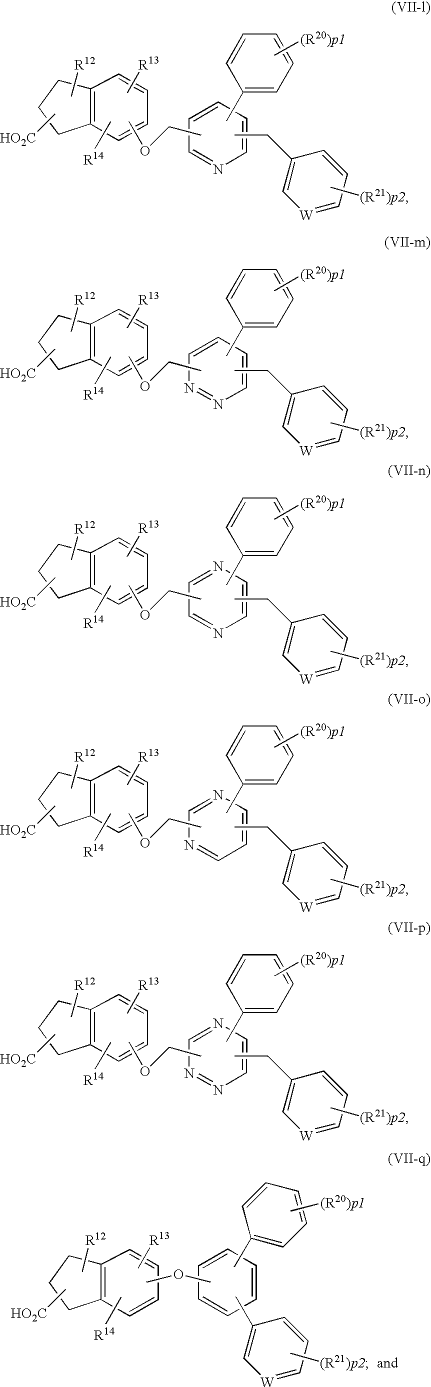

wherein the (R²⁰)ₚ₁-aryl and (R²¹)ₚ₂-(hetero)aryl moieties are attached to two contiguous central ring atoms.

In preferred embodiments, the compounds of the present invention have structural Formula (VIII):

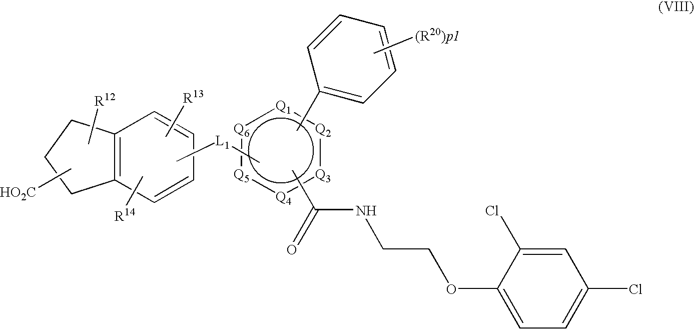

wherein:

L¹ from the group consisting of a bond, —CH₂O—, and —OCH₂—;

R¹², R¹³ and R¹⁴ are independently selected from the group consisting of hydrogen, lower alkyl, lower alkoxy and halogen;

R²² is selected from the group consisting of hydrogen, methoxy, isopropyloxy, trifluoromethoxy, fluoro, chloro, trifluoromethyl, methyl, and nitro; and

p1 is an integer from 0 to 2.

In preferred embodiments, the compounds of the present invention have structural Formula VIII selected from the group consisting of:

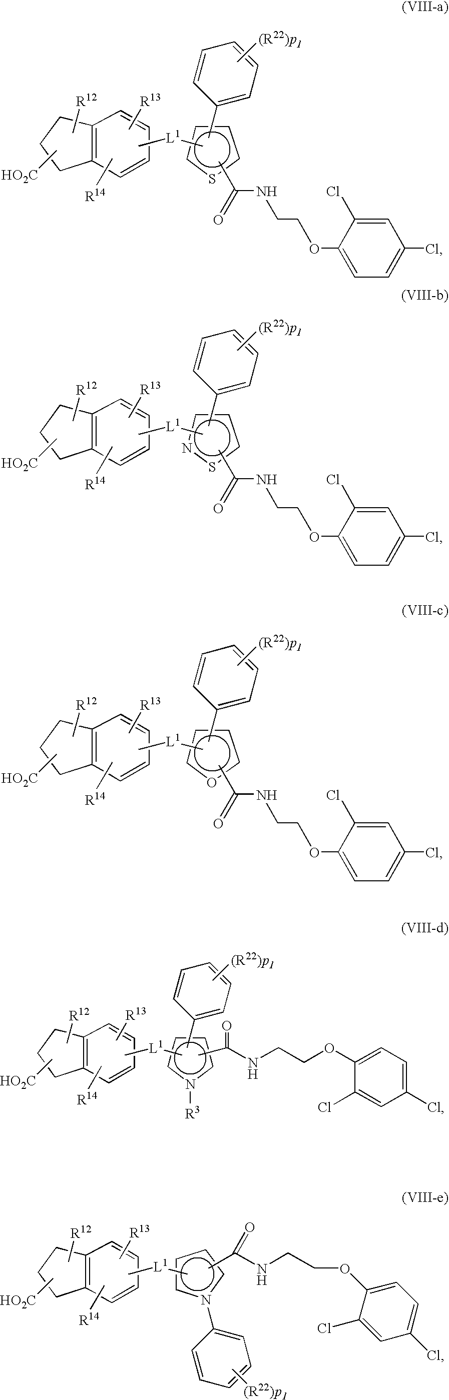

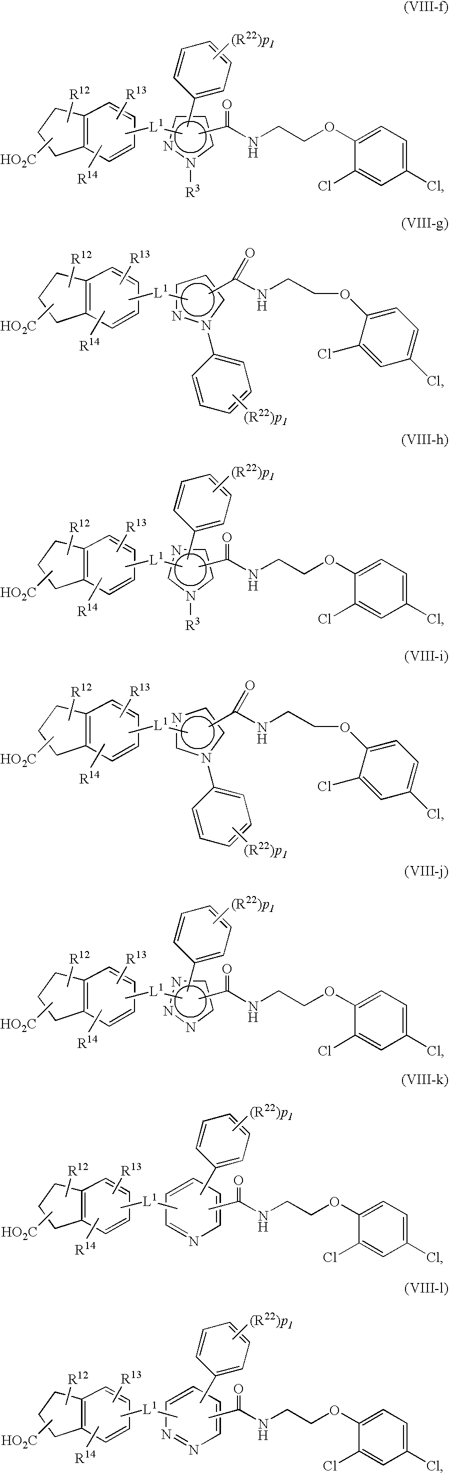

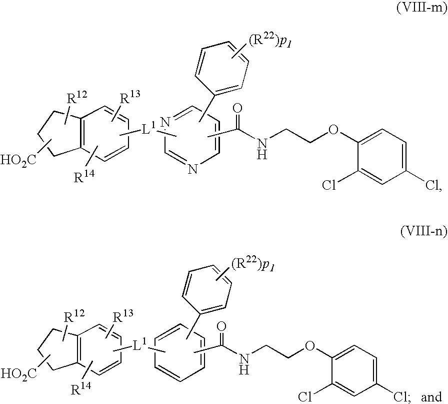

wherein the three substituents are attached to three contiguous ring atoms of the central ring moiety.

As used herein, the terms below have the meanings indicated.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. Examples of acyl groups include formyl, alkanoyl and aroyl radicals.

The term “acylamino” embraces an amino radical substituted with an acyl group. An example of an “acylamino” radical is acetylamino (CH₃C(O)NH—).

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkoxyalkoxy,” as used herein, alone or in combination, refers to one or more alkoxy groups attached to the parent molecular moiety through another alkoxy group. Examples include ethoxyethoxy, methoxypropoxyethoxy, ethoxypentoxyethoxyethoxy and the like.

The term “alkoxyalkyl,” as used herein, alone or in combination, refers to an alkoxy group attached to the parent molecular moiety through an alkyl group. The term “alkoxyalkyl” also embraces alkoxyalkyl groups having one or more alkoxy groups attached to the alkyl group, that is, to form monoalkoxyalkyl and dialkoxyalkyl groups.

The term “alkoxycarbonyl,” as used herein, alone or in combination, refers to an alkoxy group attached to the parent molecular moiety through a carbonyl group. Examples of such “alkoxycarbonyl” groups include methoxycarbonyl, ethoxycarbonyl, propoxycarbonyl, butoxycarbonyl and hexyloxycarbonyl.

The term “alkoxycarbonylalkyl” embraces radicals having “alkoxycarbonyl”, as defined above substituted to an alkyl radical. More preferred alkoxycarbonylalkyl radicals are “lower alkoxycarbonylalkyl” having lower alkoxycarbonyl radicals as defined above attached to one to six carbon atoms. Examples of such lower alkoxycarbonylalkyl radicals include methoxycarbonylmethyl.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an amino group attached to the parent molecular moiety through an alkyl group.

The term “alkylaminocarbonyl” as used herein, alone or in combination, refers to an alkylamino group attached to the parent molecular moiety through a carbonyl group. Examples of such radicals include N-methylaminocarbonyl and N,N-dimethylcarbonyl.

The term “alkylcarbonyl” and “alkanoyl,” as used herein, alone or in combination, refers to an alkyl group attached to the parent molecular moiety through a carbonyl group. Examples of such groups include methylcarbonyl and ethylcarbonyl.

The term “alkylidene,” as used herein, alone or in combination, refers to an alkenyl group in which one carbon atom of the carbon-carbon double bond belongs to the moiety to which the alkenyl group is attached.

The term “alkylsulfinyl,” as used herein, alone or in combination, refers to an alkyl group attached to the parent molecular moiety through a sulfinyl group. Examples of alkylsulfinyl groups include methylsulfinyl, ethylsulfinyl, butylsulfinyl and hexylsulfinyl.

The term “alkylsulfonyl,” as used herein, alone or in combination, refers to an alkyl group attached to the parent molecular moiety through a sulfonyl group. Examples of alkylsulfinyl groups include methanesulfonyl, ethanesulfonyl, tert-butanesulfonyl, and the like.

The term “alkylthio,” as used herein, alone or in combination, refers to an alkyl thioether (R—S—) radical wherein the term alkyl is as defined above. Examples of suitable alkyl thioether radicals include methylthio, ethylthio, n-propylthio, isopropylthio, n-butylthio, iso-butylthio, sec-butylthio, tert-butylthio, ethoxyethylthio, methoxypropoxyethylthio, ethoxypentoxyethoxyethylthio and the like.

The term “alkylthioalkyl” embraces alkylthio radicals attached to an alkyl radical. Alkylthioalkyl radicals include “lower alkylthioalkyl” radicals having alkyl radicals of one to six carbon atoms and an alkylthio radical as described above. Examples of such radicals include methylthiomethyl.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl, and the like.

The term “amido,” as used herein, alone or in combination, refers to an amino group as described below attached to the parent molecular moiety through a carbonyl group. The term “C-amido” as used herein, alone or in combination, refers to a —C(═O)—NR₂ group with R as defined herein. The term “N-amido” as used herein, alone or in combination, refers to a RC(═O)NH— group, with R as defined herein.

The term “amino,” as used herein, alone or in combination, refers to —NRR′, wherein R and R′ are independently selected from the group consisting of hydrogen, alkenyl, alkoxy, alkoxyalkyl, alkoxycarbonyl, alkyl, alkylcarbonyl, aryl, arylalkenyl, arylalkyl, cycloalkyl, haloalkylcarbonyl, heteroaryl, heteroarylalkenyl, heteroarylalkyl, heterocycle, heterocycloalkenyl, and heterocycloalkyl, wherein the aryl, the aryl part of the arylalkenyl, the arylalkyl, the heteroaryl, the heteroaryl part of the heteroarylalkenyl and the heteroarylalkyl, the heterocycle, and the heterocycle part of the heterocycloalkenyl and the heterocycloalkyl can be optionally substituted with one, two, three, four, or five substituents independently selected from the group consisting of alkenyl, alkoxy, alkoxyalkyl, alkyl, cyano, halo, haloalkoxy, haloalkyl, hydroxy, hydroxy-alkyl, nitro, and oxo.

The term “aminoalkyl,” as used herein, alone or in combination, refers to an amino group attached to the parent molecular moiety through an alkyl group. Examples include aminomethyl, aminoethyl and aminobutyl. The term “alkylamino” denotes amino groups which have been substituted with one or two alkyl radicals. Suitable “alkylamino” groups may be mono- or dialkylated, forming groups such as, for example, N-methylamino, N-ethylamino, N,N-dimethylamino, N,N-diethylamino and the like.

The terms “aminocarbonyl” and “carbamoyl,” as used herein, alone or in combination, refer to an amino-substituted carbonyl group, wherein the amino group can be a primary or secondary amino group containing substituents selected from alkyl, aryl, aralkyl, cycloalkyl, cycloalkylalkyl radicals and the like.

The term “aminocarbonylalkyl,” as used herein, alone or in combination, refers to an aminocarbonyl radical attached to an alkyl radical, as described above. An example of such radicals is aminocarbonylmethyl. The term “amidino” denotes an —C(NH)NH₂ radical. The term “cyanoamidino” denotes an —C(N—CN)NH₂ radical.

The term “aralkenyl” or “arylalkenyl,” as used herein, alone or in combination, refers to an aryl group attached to the parent molecular moiety through an alkenyl group.

The term “aralkoxy” or “arylalkoxy,” as used herein, alone or in combination, refers to an aryl group attached to the parent molecular moiety through an alkoxy group.

The term “aralkyl” or “arylalkyl,” as used herein, alone or in combination, refers to an aryl group attached to the parent molecular moiety through an alkyl group.

The term “aralkylamino” or “arylalkylamino,” as used herein, alone or in combination, refers to an arylalkyl group attached to the parent molecular moiety through a nitrogen atom, wherein the nitrogen atom is substituted with hydrogen.

The term “aralkylidene” or “arylalkylidene,” as used herein, alone or in combination, refers to an aryl group attached to the parent molecular moiety through an alkylidene group

The term “aralkylthio” or “arylalkylthio,” as used herein, alone or in combination, refers to an arylalkyl group attached to the parent molecular moiety through a sulfur atom.

The term “aralkynyl” or “arylalkynyl,” as used herein, alone or in combination, refers to an aryl group attached to the parent molecular moiety through an alkynyl group.

The term “aralkoxycarbonyl,” as used herein, alone or in combination, refers to a radical of the formula aralkyl-O—C(O)— in which the term “aralkyl,” has the significance given above. Examples of an aralkoxycarbonyl radical are benzyloxycarbonyl (Z or Cbz) and 4-methoxyphenylmethoxycarbonyl (MOS).

The term “aralkanoyl,” as used herein, alone or in combination, refers to an acyl radical derived from an aryl-substituted alkanecarboxylic acid such as benzoyl, phenylacetyl, 3-phenylpropionyl(hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like. The term “aroyl” refers to an acyl radical derived from an arylcarboxylic acid, “aryl” having the meaning given below. Examples of such aroyl radicals include substituted and unsubstituted benzoyl or napthoyl such as benzoyl, 4-chlorobenzoyl, 4-carboxybenzoyl, 4-(benzyloxycarbonyl)benzoyl, 1-naphthoyl, 2-naphthoyl, 6-carboxy-2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylamino” as used herein, alone or in combination, refers to an aryl group attached to the parent moiety through an amino group, such as methylamino, N-phenylamino, and the like.

The terms “arylcarbonyl” and “aroyl,” as used herein, alone or in combination, refer to an aryl group attached to the parent molecular moiety through a carbonyl group.

The term “aryloxy,” as used herein, alone or in combination, refers to an aryl group attached to the parent molecular moiety through an oxygen atom.

The term “arylsulfonyl,” as used herein, alone or in combination, refers to an aryl group attached to the parent molecular moiety through a sulfonyl group.

The term “arylthio,” as used herein, alone or in combination, refers to an aryl group attached to the parent molecular moiety through a sulfur atom.

The terms “carboxy” or “carboxyl”, whether used alone or with other terms, such as “carboxyalkyl”, denotes —CO₂H.

The terms “benzo” and “benz,” as used herein, alone or in combination, refer to the divalent radical C₆H₄═ derived from benzene. Examples include benzothiophene and benzimidazole.

The term “O-carbamyl” as used herein, alone or in combination, refers to a —OC(O)NR, group-with R as defined herein.

The term “N-carbamyl” as used herein, alone or in combination, refers to a ROC(O)NH— group, with R as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by bicyclo[2,2,2]octane, bicyclo[2,2,2]octane, bicyclo[1,1,1]pentane, camphor and bicyclo[3,2,1]octane.e term “cycloalkyl” embraces radicals having three to ten carbon atoms, such as cyclopropyl cyclobutyl, cyclopentyl, cyclohexyl and cycloheptyl.

The term “ester,” as used herein, alone or in combination, refers to a carbonyl group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a halohydrocarbyl group attached at two or more positions. Examples include fluoromethylene (—CFH—), difluoromethylene (—CF₂—), chloromethylene (—CHCl—) and the like. Examples of such haloalkyl radicals include chloromethyl, 1-bromoethyl, fluoromethyl, difluoromethyl, trifluoromethyl, 1,1,1-trifluoroethyl, perfluorodecyl and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH2-NH—OCH3.

The term “heteroaryl,” as used herein, alone or in combination, refers to 3 to 7 membered, preferably 5 to 7 membered, unsaturated heterocyclic rings wherein at least one atom is selected from the group consisting of O, S, and N. Heteroaryl groups are exemplified by: unsaturated 3 to 7 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl [e.g., 4H-1,2,4-triazolyl, 1H-1,2,3-triazolyl, 2H-1,2,3-triazolyl, etc.]tetrazolyl [e.g. 1H-tetrazolyl, 2H-tetrazolyl, etc.], etc.; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl, etc.], etc.; unsaturated 3 to 6-membered heteromonocyclic groups containing an oxygen atom, for example, pyranyl, furyl, etc.; unsaturated 3 to 6-membered heteromonocyclic groups containing a sulfur atom, for example, thienyl, etc.; unsaturated 3- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.]etc.; unsaturated condensed heterocyclic groups containing 1 to 2 oxygen atoms and 1 to 3 nitrogen atoms [e.g. benzoxazolyl, benzoxadiazolyl, etc.]; unsaturated 3 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl, etc.]and isothiazolyl; unsaturated condensed heterocyclic groups containing 1 to 2 sulfur atoms and 1 to 3 nitrogen atoms [e.g., benzothiazolyl, benzothiadiazolyl, etc.]and the like. The term also embraces radicals where heterocyclic radicals are fused with aryl radicals. Examples of such fused bicyclic radicals include benzofuryl, benzothienyl, and the like.

The term “heteroaralkenyl” or “heteroarylalkenyl,” as used herein, alone or in combination, refers to a heteroaryl group attached to the parent molecular moiety through an alkenyl group.

The term “heteroaralkoxy” or “heteroarylalkoxy,” as used herein, alone or in combination, refers to a heteroaryl group attached to the parent molecular moiety through an alkoxy group.

The term “heteroalkyl” or “heteroarylalkyl,” as used herein, alone or in combination, refers to a heteroaryl group attached to the parent molecular moiety through an alkyl group.

The term “heteroaralkylidene” or “heteroarylalkylidene,” as used herein, alone or in combination, refers to a heteroaryl group attached to the parent molecular moiety through an alkylidene group.

The term “heteroaryloxy,” as used herein, alone or in combination, refers to a heteroaryl group attached to the parent molecular moiety through an oxygen atom.

The term “heteroarylsulfonyl,” as used herein, alone or in combination, refers to a heteroaryl group attached to the parent molecular moiety through a sulfonyl group.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “heterocycloalkenyl,” as used herein, alone or in combination, refers to a heterocycle group attached to the parent molecular moiety through an alkenyl group.

The term “heterocycloalkoxy,” as used herein, alone or in combination, refers to a heterocycle group attached to the parent molecular group through an oxygen atom.

The term “heterocycloalkyl,” as used herein, alone or in combination, refers to an alkyl radical as defined above in which at least one hydrogen atom is replaced by a heterocyclo radical as defined above, such as pyrrolidinylmethyl, tetrahydrothienylmethyl, pyridylmethyl and the like.

The term “heterocycloalkylidene,” as used herein, alone or in combination, refers to a heterocycle group attached to the parent molecular moiety through an alkylidene group.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl” as used herein, alone or in combination, refers to a linear or branched alkyl group having one to about ten carbon atoms any one of which may be substituted with one or more hydroxyl radicals. Examples of such radicals include hydroxymethyl, hydroxyethyl, hydroxypropyl, hydroxybutyl and hydroxyhexyl.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptoalkyl” as used herein, alone or in combination, refers to an R′SR— group, where R and R′ are as defined herein.

The term “mercaptomercaptyl” as used herein, alone or in combination, refers to a RSR′S— group, where R is as defined herein.

The term “mercaptyl” as used herein, alone or in combination, refers to an RS— group, where R is as defined herein.

The term “null” refers to a lone electron pair.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “oxo” as used herein, alone or in combination, refers to a doubly bonded oxygen.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S and —S—.

The term “sulfinyl,” as used herein, alone or in combination, refers to —S(O)—.

The term “sulfonyl,” as used herein, alone or in combination, refers to —SO₂—.

The term “N-sulfonamido” refers to a RS(═O)₂NH— group with R as defined herein.

The term “S-sulfonamido” refers to a —S(═O)₂NR₂, group, with R as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thioether,” as used herein, alone or in combination, refers to a thio group bridging two moieties linked at carbon atoms.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NH— group, with R as defined herein.

The term “O-thiocarbamyl” refers to a —OC(S)NR, group with R as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethylsilyl, tert-butyldimethylsilyl, triphenylsilyl and the like.

The term “optionally substituted” means the anteceding group may be substituted or unsubstituted. When substituted, the substituents of an “optionally substituted” group may include, without limitation, one or more substituents independently selected from the following groups or designated subsets thereof, alone or in combination: hydrogen, carbonyl(oxo), carboxyl, lower alkyl carboxylate, lower alkyl carbonate, lower alkyl carbamate, halogen, hydroxy, amino, amido, cyano, hydrazinyl, hydrazinylcarbonyl, alkylhydrazinyl, dialkylhydrazinyl, arylhydrazinyl, heteroarylhydrazinyl, thiol, sulfonic acid, trisubstituted silyl, urea, acyl, acyloxy, acylamino, acylthio, lower alkyl, lower alkylamino, lower dialkylamino, lower alkyloxy, lower alkoxyalkyl, lower alkylthio, lower alkylsulfonyl, lower alkenyl, lower alkenylamino, lower dialkenylamino, lower alkenyloxy, lower alkenylthio, lower alkenyl sulfonyl, lower alkynyl, lower alkynylamino, lower dialkynylamino, lower alkynyloxy, lower alkynylthio, lower alkynylsulfonyl, lower cycloalkyl, lower cycloalkyloxy, lower cycloalkylamino, lower cycloalkylthio, lower cycloalkylsulfonyl, lower cycloalkylalkyl lower cycloalkylalkyloxy, lower cycloalkylalkylamino, lower cycloalkylalkylthio, lower cycloalkylalkylsulfonyl, aryl, aryloxy, arylamino, arylthio, arylsulfonyl, arylalkyl, arylalkyloxy, arylalkylamino, arylalkylthio, arylalkylsulfonyl, heteroaryl, heteroaryloxy, heteroarylamino, heteroarylthio, heteroarylsulfonyl, heteroarylalkyl, heteroarylalkyloxy, heteroarylalkylamino, heteroarylalkylthio, heteroarylalkylsulfonyl, heterocycloalkyl, heterocycloalkyloxy, heterocycloalkylamino, heterocycloalkylthio, heterocycloalkylsulfonyl, lower haloalkyl, lower haloalkenyl, lower haloalkynyl, lower perhaloalkyl, lower perhaloalkoxy, lower haloalkoxy, and lower acyloxy. Two substituents may be joined together to form a fused four-, five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. All pendant aryl, heteroaryl, and heterocyclo moieties can be further optionally substituted with one, two, three, four, or five substituents independently selected from the groups listed above.

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PPAR modulator” is used herein to refer to a compound that exhibits an EC₅₀ with respect to PPAR activity of no more than about 100 μM and more typically not more than about 50 μM, as measured in the PPAR transcriptional assay described generally hereinbelow. “EC₅₀” is that concentration of inhibitor which reduces the activity of an enzyme (e.g., PPARs) to half-maximal level. Representative compounds of the present invention have been discovered to exhibit inhibitory activity against PPARs, in particular PPARδ. Compounds of the present invention preferably exhibit an EC₅₀ with respect to PPARδ of no more than about 10 μM, more preferably, no more than about 5 μM, even more preferably not more than about 1 μM, and most preferably, not more than about 200 nM, as measured in the PPAR transcriptional assay described herein.

The phrase “therapeutically effective” is intended to qualify the amount of active ingredients used in the treatment of dyslipidemia, hyperlipidemia, hypercholesteremia, metabolic syndrome X, atherosclerosis, atherogenesis, heart failure, myocardial infarction, vascular diseases, cardiovascular disease, type II diabetes mellitus, type 1 diabetes, insulin resistance, hypertension, obesity, anorexia bulimia, anorexia nervosa, hair growth abnormalities, skin disorders, inflammation, arthritis, cancer, Alzheimer's disease, respiratory diseases, ophthalmic disorders, IBDs (irritable bowel disease), ulcerative colitis and Crohn's disease. This amount will achieve the goal of reducing or eliminating the pathological condition

The term “prodrug” refers to a compound that is made more active in vivo. The present compounds can also exist as prod rugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate(besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethanesulfonate(isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention with one or more therapeutic agents (pharmaceutical combinations).

Thus, the present invention also relates to pharmaceutical combinations, such as a combined preparation or pharmaceutical composition (fixed combination), comprising a compound of the invention as defined above or a pharmaceutically acceptable salt thereof; and at least one active ingredient selected from:

a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-¹⁹4²⁰4; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; DPPIV (dipeptidyl peptidase IV) inhibitors such as DPP728, LAF237 (vildagliptin—Example 1 of WO 00/34241), MK-0431, saxagliptin, GSK23A; an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}2,3-dihydro-1H-indole-2-carboxylic acid described in the patent application WO 03/043985, as compound 19 of Example 4, a non-glitazone type PPARδ agonist e.g. GI-262570;

b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (famesoid X receptor) and LXR (liver X receptor) ligands; cholestyramine; fibrates; nicotinic acid and aspirin;

c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;

d) anti-hypertensive agents, e.g., loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutral endopeptidase (NEP) inhibitors e.g. thiorphan, terteothiorphan, SQ29072; ECE inhibitors e.g. SLV306; ACE/NEP inhibitors such as omapatrilat, sampatrilat and fasidotril; angiotensin n antagonists such as candesartan, eprosartan, irbesartan, losartan, tehnisartan and valsartan, in particular valsartan; renin inhibitors such as aliskiren, terlakiren, ditekiren, RO 66-1132, RO-66-1¹⁶8; □-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;

e) a HDL increasing compound;

f) Cholesterol absorption modulator such as Zetia® and KT6-971;

g) Apo-A1 analogues and mimetics;

h) thrombin inhibitors such as Ximelagatran;

i) aldosterone inhibitors such as anastrazole, fadrazole, epierenone;

j) Inhibitors of platelet aggregation such as aspirin, clopidogrel bisulfate;

k) estrogen, testosterone, a selective estrogen receptor modulator, a selective androgen receptor modulator;

l) a chemotherapeutic agent such as aromatase inhibitors e.g. femara, anti-estrogens, topoisomerase I inhibitors, topoisomerase 11 inhibitors, microtubule active agents, alkylating agents, antineoplastic antimetabolites, platin compounds, compounds decreasing the protein kinase activity such as a PDGF receptor tyrosine kinase inhibitor preferably miatinib ({N-{5-[4-(4-methyl-piperazino-methyl)-benzoylamido]-2-methylphenyl}-4-(3-pyridyl)-2-pyrimidine-amine}) described in the European patent application EPA-0564409 as example 21 or 4-Methyl-N-[3-(4-methyl-imidazol-1-yl)-5-trifluoromethyl-phenyl]-3-(4-pyridin-3-yl-pyrimidin-2-ylamino)-benzamide described in the patent application WO 04/005281 as example 92; and

m) an agent interacting with a 5-HT3 receptor and/or an agent interacting with 5-HT4 receptor such as tegaserod described in the U.S. Pat. No. 5,510,353 as example 13, tegaserod hydrogen maleate, cisapride, cilansetron;

or, in each case a pharmaceutically acceptable salt thereof, and optionally a pharmaceutically acceptable carrier.

Most preferred combination partners are tegaserod, imatinib, vildagliptin, metformin, a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid, a sulfonylurea receptor ligand, aliskiren, valsartan, oriistat or a statin such as pitavastatin, simvastatin, fluvastatin or pravastatin.

In another preferred aspect the invention concerns a pharmaceutical composition (fixed combination) comprising a therapeutically effective amount of a compound as described herein, in combination with a therapeutically effective amount of at least one active ingredient selected from the above described group a) to m), or, in each case a pharmaceutically acceptable salt thereof.

A pharmaceutical composition or combination as described herein for the manufacture of a medicament for the treatment of for the treatment of dyslipidemia, hyperiipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.

Such therapeutic agents include estrogen, testosterone, a selective estrogen receptor modulator, a selective androgen receptor modulator, insulin, insulin derivatives and mimetics; insulin secretagogues such as the sulfonylureas, e.g., Glipizide and Amaryl; insulinotropic sulfonylurea receptor ligands, such as meglitinides, e.g., nateglinide and repaglinide; insulin sensitizers, such as protein tyrosine phosphatase-1B (PTP-1B) inhibitors, GSK3 (glycogen synthase kinase-3) inhibitors or RXR ligands; biguanides, such as metformin; alpha-glucosidase inhibitors, such as acarbose; GLP-1 (glucagon like peptide-1), GLP-1 analogs, such as Exendin-4, and GLP-1 mimetics; DPPIV (dipeptidyl peptidase IV) inhibitors, e.g. isoleucin-thiazolidide; DPP728 and LAF237, hypolipidemic agents, such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin, fluindostatin and rivastatin, squalene synthase inhibitors or FXR (liver X receptor) and LXR (farnesoid X receptor) ligands, cholestyramine, fibrates, nicotinic acid and aspirin.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating PPAR-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of PPAR-mediated disorders.

The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein.

### General Synthetic Methods for Preparing Compounds

Compounds of the present invention may be synthesized using standard synthetic techniques known to those of skill in the art or using methods known in the art in combination with methods described herein. As a guide the following synthetic methods may be utilized.

A. Formation of Covalent Linkages by Reaction of an Electrophile with a Nucleophile

Selected examples of covalent linkages and precursor functional groups which yield them are given in Table 1. Precursor functional groups are shown as electrophilic groups and nucleophilic groups. The functional group on the organic substance may be attached directly, or attached via any useful spacer or linker as defined below.

TABLE 1
Examples of Covalent Linkages and Precursors Thereof.

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoborons and organoboronates). Some of these carbon nucleophiles have the advantage of being kinetically stable in water or polar organic solvents. Other carbon nucleophiles include phosphorus ylids, enol and enolate reagents. These carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, alcohols, alkoxides, azides, hydrazides, semicarbazides, and the like. These non-carbon nucleophiles, when used in conjunction with carbon electrophiles, typically generate heteroatom linkages (C—X—C), wherein X is a hetereoatom, e.g., oxygen, sulfur or nitrogen.

B. Use of Protecting Groups

In the reactions described, it may be necessary to protect reactive functional groups, for example hydroxy, amino, imino, thio or carboxy groups, where these are desired in the final product. The term “protecting group” refers to chemical moieties that block some or all such reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, hydrogenolysis, and metal-catalyzed processes. Groups such as trityl, dimethoxytrityl, acetal, ketal, t-butyl, and t-butyldimethylsilyl are acid labile and may be used to protect and deprotect carboxy, carbonyl (aldehyde, ketone), and hydroxyl moieties, e.g. in the presence of Cbz-protected amino groups, which are removable by hydrogenolysis; and Fmoc groups, which are base labile. Carboxylic acid and hydroxy moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

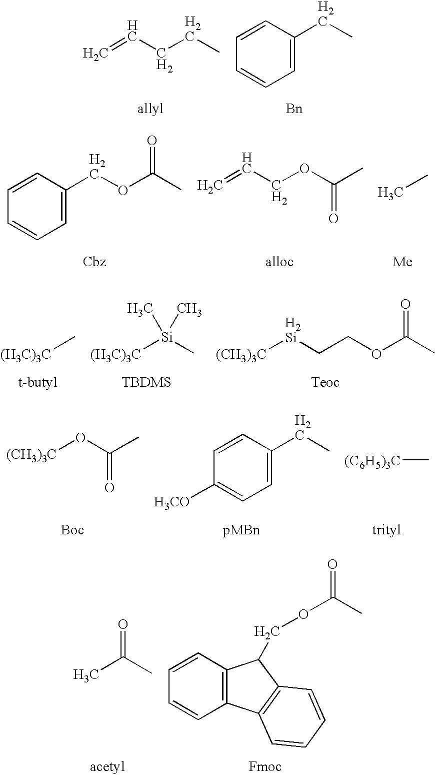

Other protecting groups are described in Greene and Wuts, Protective Groups in Organic Synthesis, 3rd Ed., John Wiley & Sons, New York, N.Y., 1999, which is incorporated herein by reference in its entirety.

C. Synthetic Approaches to Compounds

The compounds of the present invention may be synthesized using the general synthetic procedures and examples set forth in the references cited below.

### I. GENERAL REFERENCE WORKS

Comprehensive Organic Chemistry, D. H. R. Barton and W. D. Ollis, Eds., Pergamon, Oxford, UK, 1979. Vols. 1-6.
Comprehensive Organic Synthesis, B. M. Trost and I. Fleming, Eds. Pergamon, Oxford, UK 1991; Vols. 1-9.
Comprehensive Organic Functional Group Transformations II, Second Edition: A Comprehensive Review of the Synthetic Literature 1995-2000, A. R. Katritzky and R. J. K. Taylor, Eds., Elsevier Science; Oxford, UK, 2004; Vol. 1-7.
C. Buncel, Comprehensive Carbanion Chemistry, Part C, Elsevier Science, Oxford, UK, 1987.
R. C. Larock, Comprehensive Organic Transformations: A Guide to Functional Group Preparations, 2^{nd} Ed. Wiley-VCH, New York, 1999.
Comprehensive Heterocyclic Chemistry II, A. R. Katritsky, C. W. Rees, and E. F. V. Scriver, eds., Pergamon Press, Oxford, UK, 1996; Vol. 1-11.
Comprehensive Organometallic Chemistry II, E. W. Abel, F. G. A. Stone and G. Wilkinson, Eds., Pergamon; Oxford, UK, 1995; Vols. 1-14.
M. B. Smith and J. March, March's Advanced Organic Chemistry: Reactions, Mechanisms, and Structure, 5^{th} Ed., Wiley-Interscience, New York, 2001.
Advances in Heterocyclic Chemistry, A. R. Katritsky, Ed., Elsevier, Oxford, UK, 1960-2005; Vols. 1-90.
Name Reactions in Heterocyclic Chemistry, Jie Jack Li, Ed. Wiley-Interscience, New York, 2004.
T. Eicher and S. Hauptmann, The Chemistry of Heterocycles: Structure, Reactions, Syntheses, and Applications, John Wiley & Sons, New York, 2003.
R. K Bansal, Heterocyclic Chemistry, New Age International, Ltd., New Delhi, 1999.
J. A. Joule, Heterocyclic Chemistry, 4^{th} Ed., Blackwell Publishers, Oxford, UK, 2000.
T. L. Gilchrist, Heterocyclic Chemistry, 3^{rd} Ed., Longman, London, 1997.
1,3-Dipolar Cycloaddition Chemistry, A. Padwa, Ed., John Wiley and Sons, New York, 1984; Vols. 1 and 2.
Beilstein Handbook of Organic Chemistry, 4^{th} ed., Springer-Verlag, Darmstadt, 1990.
Contemporary Heterocyclic Chemistry: Syntheses, Reactions, and Applications, G. R. Newkome and W. W. Parldeh, Eds., John Wiley and Sons, New York, 1982.

### II. ISOTHIAZOLES

C. Cutri, A. Garozzo, M. A. Siracusa, M. C. Sarva, A. Castro A, E. Geremia E, M. R. Pinizzotto, and F. Guerrera, Bioorg Med. Chem., 1999, 7, 225.
Comprehensive Heterocyclic Chemistry II, A. R. Katritzky, C. W. Rees, E. F. V. Scriven, Eds., Ch. Pergamon Press, Oxford, UK 1996; Vol. 3, Ch. 5.
J. A. Joule, Heterocyclic Chemistry, 4^{th} Ed., Blackwell Publishers, Oxford, UK, 2000; Ch. 22, 431.
T. L. Gilchrist, Heterocyclic Chemistry, 3^{rd} Ed., Longman, London, 1997; Ch. 8, 328.

### III. THIOPHENES

The Chemistry of Heterocyclic Compounds: Thiophene and Its Derivatives Pt. 1, S. Gronowitz, Ed., Wiley-Interscience, New York, 1985; Vol. 43.
The Chemistry of Heterocyclic Compounds: Thiophene and Its Derivatives Pt. 2, S. Gronowitz, Ed., Wiley-Interscience, New York, 1986; Vol 44.
The Chemistry of Heterocyclic Compounds: Thiophene and Its Derivatives Pt. 3, S. Gronowitz, Ed., Wiley-Interscience, New York, 1986; Vol. 44.
S. Gronowitz and A. B. Hörnfeldt, Thiophenes: Best Synthetic Methods, Academic Press, New York, 2004.
Comprehensive Heterocyclic Chemistry II, A. R. Katritzky, C. W. Rees, E. F. V. Scriven, Eds., Pergamon Press, Oxford, UK 1996; Vol. 2, Ch. 11.
J. A. Joule, Heterocyclic Chemistry, 4^{th} Ed., Blackwell Publishers, Oxford, UK, 2000; Ch. 14, 273.
T. L. Gilchrist, Heterocyclic Chemistry, 3^{rd} Ed., Longman, London, 1997; Ch. 6, 221.

### IV. ISOXAZOLES

The Chemistry of Heterocyclic Compounds:Isoxazoles, Part 2, P. Grünanger and P. Vita-Finzi, Eds., Wiley-Interscience, New York, 1999; Vol. 49.
Comprehensive Heterocyclic Chemistry II, A. R. Katritzky, C. W. Rees, E. F. V. Scriven, Eds., Pergamon Press, Oxford, UK 1996; Vol. 3, Ch. 3.
J. A. Joule, Heterocyclic Chemistry, 4^{th} Ed., Blackwell Publishers, Oxford, UK, 2000; Ch. 22, 431.
T. L. Gilchrist, Heterocyclic Chemistry, 3^{rd} Ed., Longman, London, 1997; Ch. 8, 328.
1,3-Dipolar Cycloaddition Chemistry, A. Padwa, Ed., John Wiley and Sons, New York, 1984; Vol. 1, 338.

### V. FURANS

Comprehensive Heterocyclic Chemistry II, A. R. Katritzky, C. W. Rees, E. F. V. Scriven, Eds., Pergamon Press, Oxford, UK 1996; Vol. 2, Ch. 7.
J. A. Joule, Heterocyclic Chemistry, 4^{th} Ed., Blackwell Publishers, Oxford, UK, 2000; Ch. 15, 296.
T. L. Gilchrist, Heterocyclic Chemistry, 3^{rd} Ed., Longman, London, 1997; Ch. 6, 208.
1,3-Dipolar Cycloaddition Chemistry, A. Padwa, Ed., John Wiley and Sons, New York, 1984; Vol. 1, 694.
A. Williams, Furans:Synthesis and Applications, Noyes Data Corp., 1973.

### VI. PYRROLES

Comprehensive Heterocyclic Chemistry II, A. R. Katritzky, C. W. Rees, E. F. V. Scriven, Eds., Pergamon Press, Oxford, UK 1996; Vol. 2, Ch. 3.
J. A. Joule, Heterocyclic Chemistry, 4^{th} Ed., Blackwell Publishers, Oxford, UK, 2000; Ch. 13, 237.
T. L. Gilchrist, Heterocyclic Chemistry, 3^{rd} Ed., Longman, London, 1997; Ch. 6, 193.
The Synthesis and the Physical and Chemical Aspects of the Pyrrole Ring;Pyrroles Part 1, R. A. Jones, Ed. Wiley-Interscience, New York, 1990; Vol. 48.
The Synthesis and the Physical and Chemical Aspects of the Pyrrole Ring;Pyrroles Part 2, R. A. Jones, Ed. Wiley-Interscience, New York, 1992; Vol. 48.
R. A. Jones, The Chemistry of Pyrroles, Academic Press, New York, 1977.
A Gossauer, Die Chemie der Pyrrole: Organische Chemie in Einzeldarstellungen, Springer Verlag, Darmstadt, 1974.
1,3-Dipolar Cycloaddition Chemistry, A. Padwa, Ed., John Wiley and Sons, New York, 1984; Vol. 1, 184; vol. 2, 467.

### VII. PYRAZOLES

The Chemistry of Heterocyclic Compounds: Pyrazoles, Pyrazolines, Pyrazolidines, Indazoles, and Condensed Rings, R. H Wiley, Ed., Interscience Publishers, New York, 1967; Vol. 22.
Comprehensive Heterocyclic Chemistry II, A. R. Katritzky, C. W. Rees, E. F. V. Scriven, Eds., Pergamon Press, Oxford, UK 1996; Vol. 3, Ch. 1.
J. A. Joule, Heterocyclic Chemistry, 4^{th} Ed., Blackwell Publishers, Oxford, UK, 2000; Ch. 22, 431.
T. L. Gilchrist, Heterocyclic Chemistry, 3^{rd} Ed., Longman, London, 1997; Ch. 8, 304.
1,3-Dipolar Cycloaddition Chemistry, A. Padwa, Ed., John Wiley and Sons, New York, 1984; Vol. 1, Ch. 3 and 4; Vol. 2, 364.

### VIII. IMIDAZOLES

M. R. Grimmett, Imidazole and Benzimidazole Synthesis:Best Synthetic Methods, Academic Press, New York, 1997.
Comprehensive Heterocyclic Chemistry II, A. R. Katritzky, C. W. Rees, E. F. V. Scriven, Eds., Pergamon Press, Oxford, UK 1996; Vol. 3, Ch. 2.
J. A. Joule, Heterocyclic Chemistry, 4^{th} Ed., Blackwell Publishers, Oxford, UK, 2000; Ch. 21, 402.
T. L. Gilchrist, Heterocyclic Chemistry, 3^{rd} Ed., Longman, London, 1997; Ch. 8, 298.
The Chemistry of Heterocyclic Compounds:Imidazole and its Derivatives, K. Hofmann, Ed., Interscience Publishers, New York, 1953.

### IX. 1,2,3-TRIAZOLES AND 1,2,4-TRIAZOLES

1,3-Dipolar Cycloaddition Chemistry, A. Padwa, Ed., John Wiley and Sons, New York, 1984; Vol. 1, Ch. 5.
C. Temple and J. A. Montgomery, 1,2,4-Triazoles, John Wiley and Sons, New York, 1981.
Comprehensive Heterocyclic Chemistry II, A. R. Katritzky, C. W. Rees, E. F. V. Scriven, Eds., Pergamon Press, Oxford, UK 1996; Vol. 4, Ch. 1 and 2.
J. A. Joule, Heterocyclic Chemistry, 4^{th} Ed., Blackwell Publishers, Oxford, UK, 2000; Ch. 26, 504.
T. L. Gilchrist, Heterocyclic Chemistry, 3^{rd} Ed., Longman, London, 1997; Ch. 8, 304.

### X. PYRIDINES

Rodd's Chemistry of Carbon Compounds: Six-Membered Heterocyclic Compounds with a Single Nitrogen Atom in the Ring; Pyridine, Polymethylenepyridines, Quinoline, Isoquinoline and Their Derivatives, S. Coffey, Ed., Elsevier Science, Oxford, UK, 1976; 2^{nd} Ed.
The Chemistry of Heterocyclic Compounds, Pyridine and Its Derivatives, Pt. 5, G. R. Newkome, Ed., Wiley-Interscience, New York, 1984; Vol. 14.
The Chemistry of Heterocyclic Compounds, Pyridine and Its Derivatives, E. Klingsberg, Ed., John Wiley and Sons, New York, 1963.
A. D. Dunn and W. D. Rudorf, Pyridines (Ellis Horwood Series in Organic Chemistry: Synthetic Reagents), Prentice Hall Europe, 1994.
The Chemistry of Heterocyclic Compounds, Pyridine and Its Derivatives: Supplement, R. A Abramovitch, Ed., John Wiley, New York, 1974; Vol. 14.
Comprehensive Heterocyclic Chemistry II, A. R. Katritzky, C. W. Rees, E. F. V. Scriven, Eds., Pergamon Press, Oxford, UK 1996; Vol. 5, Ch. 5.
J. A. Joule, Heterocyclic Chemistry, 4^{th} Ed., Blackwell Publishers, Oxford, UK, 2000; Ch. 5, 71.
T. L. Gilchrist, Heterocyclic Chemistry, 3^{rd} Ed., Longman, London, 1997; Ch. 5, 125.

### X. PYRIDAZINES

The Chemistry of Heterocyclic Compounds, Pyridazines, R. N. Castle, Ed., John Wiley, New York, 1973; Vol. 57
The Chemistry of Heterocyclic Compounds, Pyridazines, Supp. 1, D. J. Brown, Ed., Wiley-Interscience, New York, 1999; Vol. 57.
Rodd's Chemistry of Carbon Compounds: Heterocyclic Compounds: Part I: Six-Membered Heterocyclic Compounds with Two Hetero-Atoms from Group V of the Periodic Table: The Pyridazine and Pyrimidine Groups, Supplements to the 2nd Edition, M. F. Ansell, Ed., Elsevier Publishing Company, Oxford, UK, 1995; 2nd edition.
Comprehensive Heterocyclic Chemistry II, A. R. Katritzky, C. W. Rees, E. F. V. Scriven, Eds., Pergamon Press, Oxford, UK 1996; Vol. 6, Ch. 1.
J. A. Joule, Heterocyclic Chemistry, 4^{th} Ed., Blackwell Publishers, Oxford, UK, 2000; Ch. 11, 194.
T. L. Gilchrist, Heterocyclic Chemistry, 3^{rd} Ed., Longman, London, 1997; Ch. 7, 276.

### XI. PYRAZINES

The Chemistry of Heterocyclic Compounds, The Pyrazines, Supp. I, D. J. Brown, Ed., Wiley-Interscience; New York, 2001; Vol. 58.
The Chemistry of Heterocyclic Compounds: The Pyrazines, G. B Barlin, Ed., John Wiley, New York, 1982; Vol. 41.
Comprehensive Heterocyclic Chemistry II, A. R. Katritzky, C. W. Rees, E. F. V. Scriven, Eds., Pergamon Press, Oxford, UK 1996; Vol. 6, Ch. 3.
J. A. Joule, Heterocyclic Chemistry, 4^{th} Ed., Blackwell Publishers, Oxford, UK, 2000; Ch. 11, 194.
T. L. Gilchrist, Heterocyclic Chemistry, 3^{rd} Ed., Longman, London, 1997; Ch. 7, 276.

### XII. PYRIMIDINES

The Chemistry of Heterocyclic Compounds, The Pyrimidines, D. J. Brown, Ed., John Wiley and Sons, New York, 1994; Vol. 52.
Rodd's Chemistry of Carbon Compounds: Heterocyclic Compounds: Part I: Six-Membered Heterocyclic Compounds with Two Hetero-Atoms from Group V of the Periodic Table: The Pyridazine and Pyrimidine Groups, Supplements to the 2nd Edition, M. F. Ansell, Ed., Elsevier Publishing Company, Oxford, UK, 1995; 2nd edition.
Comprehensive Heterocyclic Chemistry II, A. R. Katritzky, C. W. Rees, E. F. V. Scriven, Eds., Pergamon Press, Oxford, UK 1996; Vol. 6, Ch. 2.
J. A. Joule, Heterocyclic Chemistry, 4^{th} Ed., Blackwell Publishers, Oxford, UK, 2000; Ch. 11, 194.
T. L. Gilchrist, Heterocyclic Chemistry, 3^{rd} Ed., Longman, London, 1997; Ch. 7, 261.

### XIII. 1,2,4-TRIAZINES AND 1,3,5-TRIAZINES

H. Neunhoeffer and P. F. Wiley, Chemistry of 1,2,3-Triazines and 1,2,4-Triazines, Tetrazines and Pentazines, John Wiley & Sons, New York, 1978, 189.
Comprehensive Heterocyclic Chemistry II, A. R. Katritzky, C. W. Rees, E. F. V. Scriven, Eds., Pergamon Press, Oxford, UK 1996; Vol. 6, Ch. 11 and 12.
J. A. Joule, Heterocyclic Chemistry, 4^{th} Ed., Blackwell Publishers, Oxford, UK, 2000; Ch. 26, 504.
T. L. Gilchrist, Heterocyclic Chemistry, 3^{rd} Ed., Longman, London, 1997; Ch. 7, 276.
1,3-Dipolar Cycloaddition Chemistry, A. Padwa, Ed., John Wiley and Sons, New York, 1984; Vol. 1, Ch. 5.

### XIV. CARBOCYCLIC ARYL COMPOUNDS

Rodd's Chemistry of Carbon Compounds 2nd Edition, Supplements to the 2nd Edition of Volume 3: Aromatic Compounds, Martin F. Ansell, Ed., Elsevier Science Ltd. Oxford, UK, 1982.
Rodd's Chemistry of Carbon Compounds 2nd Edition, Supplements to the 2nd Edition of Volume 3: Aromatic Compounds, Martin F. Ansell, Ed., Elsevier Science Ltd. Oxford, UK, 1988.
H. Zollinger, Aromatic Compounds, University Park Press, Baltimore, 1973.
F. Terrier, Nucleophilic Aromatic Displacement: The Influence of the NitroGroup, John Wiley and Sons, New York, 1991.
Rodd's Chemistry of Carbon Compounds. 2nd Edition, Aromatic Compounds:Part G: Monocarboxylic Acids of the Benzene Series, S. Coffey, Ed., Elsevier Science, Oxford, UK, 1978.
Rodd's Chemistry of Carbon Compound, 2nd Edition, Aromatic Compounds Part A: General Introduction; Mononuclear Hydrocarbons and Their Halogeno Derivatives; and Derivatives with Nuclear Substituents Attached, S. Coffey, Ed., Elsevier Science Oxford, UK, 1971.
Rodd's Chemistry of Carbon Compound, 2nd Edition, Aromatic Compounds Part C: Nuclear-Substituted Benzenoid Hydrocarbons with More Than One Nitrogen Atom in a Substituent Group, S. Coffey, Ed., Elsevier Science Oxford, UK, 1973.
D. R. Astruc, Modern Arene Chemistry: Concepts, Synthesis, and Applications, Wiley-VCH, New York, 2002.
J. H. P. Tyman, Synthetic and Natural Phenols, Elsevier Science, Oxford, UK, 1996.
Alkenes and Aromatics, P. G. Taylor and J. M. F. Gagan, Eds., Royal Society of Chemistry; London, 2002.
O. N. Chupakhin, V. N. Charushin, H. C. van der Plas, Nucleophilic Aromatic Substitution of Hydrogen, Academic Press, New York, 1994.
Chemistry of Functional Groups: The Chemistry of Phenols, Z. Rappoport, Ed., John Wiley and Sons, New York, 2003.
R. A. Rossi and R. H. de Rossi, Aromatic Substitution by the SRN1 Mechanism (ACS Monograph Series), American Chemical Society Publications, Washington, D.C., 1983.
R. Taylor, Electrophilic Aromatic Substitution, John Wiley and Sons, New York, 1990.
K. Schofield, Aromatic Nitration, Cambridge University Press, Cambridge, UK, 1981.
Friedel-Crafts and Related Reactions, G. A. Olah, Ed., John Wiley and Sons Inc., New York, 1963.
George A. Olah, Friedel-Crafts Chemistry, John Wiley and Sons Inc., New York, 1973.
   D. Synthetic Schemes

Compounds of the present invention may be synthesized using the general synthetic procedures and examples set forth below. Starting materials are commercially available, are made by known procedures, or are prepared as illustrated herein.

A principal route for preparation of compounds within the scope of the instant invention is depicted in Scheme 1:

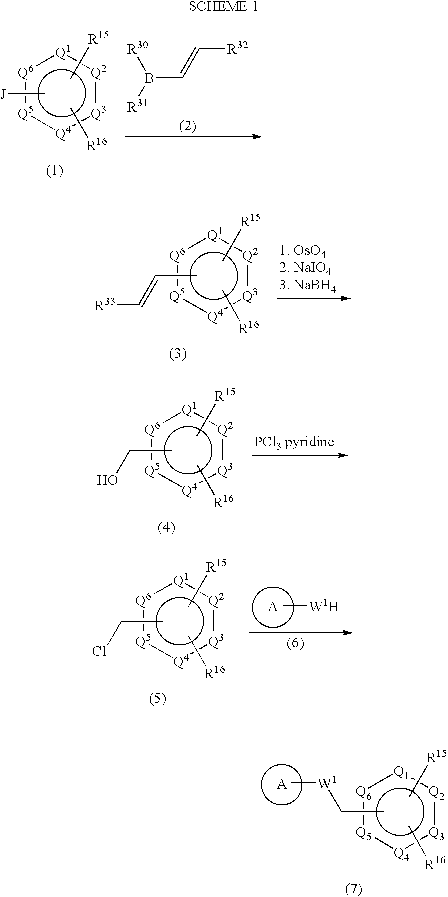

According to this route, coupling of intermediate 1 (J=bromine, iodine, chlorine, trifluoromethanesulfonate) with the organoboron derivative 2 (R³⁰ and R³¹ are independently chosen from lower alkoxy, lower alkyl, cycloalkyl; or R³⁰ and R³¹, together with the atoms to which they are attached, may be joined to form an optionally substituted heterocycloalkyl or optionally substituted cycloalkyl moiety; R³²=H, lower alkyl, aryl) affords olefin derivative 3. The reaction is conducted in the presence of a suitable catalyst (e.g., Pd(Ph₃)₄, or the like), a suitable base (e.g., Na₂CO₃, or the like) and a suitable solvent (e.g., water, ethanol, DME or the like). The reaction is carried out in the temperature range of about 80° to about 200° C., optionally in a microwave reactor. (A. Suzuki, Chem. Commun., 2005, 38, 4759; S. Schroeter, C. Stock, T. Bach, Tetrahedron, 2005, 61, 2245; J. Hassan, M. Sevignon, C. Gozzi, E. Schulz, M. Lemaire, Chem. Rev., 2002, 102, 1359). Bis-hydroxylation of olefin 3 with a catalytic amount of osmium tetroxide in the presence of a suitable co-oxidant (e.g. N-methylmorpholine oxide or the like), in a suitable solvent (e.g. dichloromethane, ethyl acetate, tetrahydrofuran, or the like), followed by diol cleavage with sodium periodate, in a suitable solvent (e.g. aqueous ethanol, methanol mixtures, or the like), and reduction of the resultant aldehyde intermediate with a reagent like sodium borohydride in a suitable solvent (e.g., ethanol, isopropranol, tert-butanol, or the like) gives alcohol 4. Alcohol 4 is treated with a suitable halogenating agent (e.g. phosphorous trichloride or the like) in the presence of a suitable base (e.g. pyridine, collidine, lutidine or the like) in a suitable solvent (e.g. diethyl ether, tetrahydrofuran, dichloromethane or the like) to give the chloro-derivative 5. Reaction of 6 (W¹=O, S) with intermediate 5 in the presence of a suitable base (e.g. sodium hydride, sodium ethoxide, potassium carbonate, N,N-diisopropylethylamine, trialkylamine bases, or the like) in a suitable solvent (e.g. acetonitrile, ethanol, tetrahydrofuran, acetone, N,N-dimethylformamide, N,N-dimethylacetamide, or the like) affords PPAR modulator 7 (Formula I, wherein L¹=—XOX—, —XSX—). These compounds can exist as mixtures of stereoisomers. These compounds can be separated by a variety of methods, including by HPLC using a column with a chiral stationary phase.

Another principal route for preparation of compounds within the scope of the instant invention is depicted in Scheme 2:

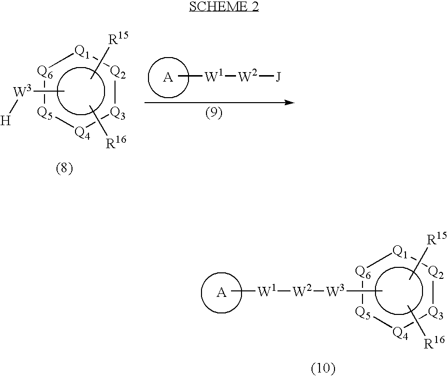

According to this scheme, reaction of intermediate 8 (W³=O, S) with intermediate 9 (W¹=O, S, bond; W²=bond, lower alkyl; J=bromine, iodine, chlorine, trifluoromethanesulfonate) in the presence of a suitable base (e.g. sodium hydride, sodium ethoxide, potassium carbonate, N,N-diisopropylethylamine, trialkylamine bases or the like) in a suitable solvent (e.g. acetonitrile, ethanol, tetrahydrofuran, acetone, N,N-dimethylformamide, N,N-dimethylacetamide or the like) affords PPAR modulators 10 (Formula I, wherein L¹=—XOX—, —XSX—, —XOXOX—, —XSXOX—, —XOXSX—). These compounds can exist as mixtures of stereoisomers. These compounds can be separated by a variety of methods, including by HPLC using a column with a chiral stationary phase.

A principal route for preparation of compounds of Formula I of the instant invention is depicted in Scheme 3:

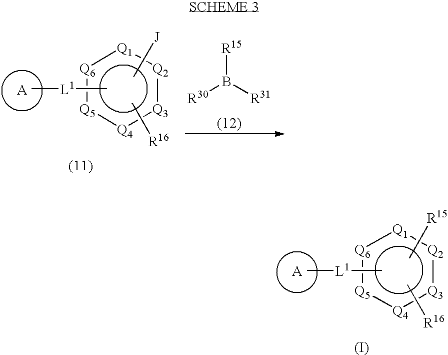

According to this scheme, reaction of intermediate 11 with the organoboron derivative 12 (R³⁰ and R³¹ are independently chosen from lower alkoxy, lower alkyl, cycloalkyl; or R³⁰ and R³¹, together with the atoms to which they are attached, may be joined to form an optionally substituted heterocycloalkyl or optionally substituted cycloalkyl moiety; R¹⁵=as defined herein) affords PPAR modulators of Formula I. The reaction is conducted in the presence of a suitable catalyst (e.g., Pd(Ph₃)₄, or the like), a suitable base (e.g., Na₂CO₃, or the like) and a suitable solvent (e.g., water, ethanol, DME or the like). The reaction is carried out in the temperature range of about 80° to about 200° C., optionally in a microwave reactor.

A principal route for preparation of compounds of Formula I of the instant invention is depicted in Scheme 4:

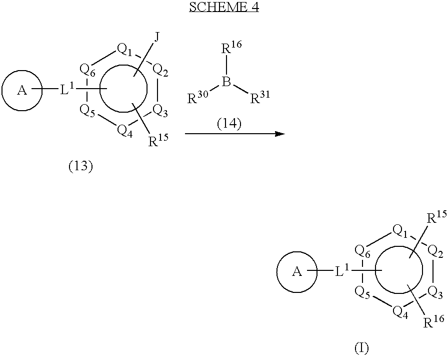

According to this scheme, reaction of intermediate 13 with the organoboron derivative 14 (R³⁰ and R³¹ are independently chosen from lower alkoxy, lower alkyl, cycloalkyl; or R³⁰ and R³¹, together with the atoms to which they are attached, may be joined to form an optionally substituted heterocycloalkyl or optionally substituted cycloalkyl moiety; R¹⁶=as defined herein) affords PPAR modulators of Formula I. The reaction is conducted in the presence of a suitable catalyst (e.g., Pd(Ph₃)₄, or the like), a suitable base (e.g., Na₂CO₃, or the like) and a suitable solvent (e.g., water, ethanol, DME or the like). The reaction is carried out in the temperature range of about 80° to about 200° C., optionally in a microwave reactor.

Another route for preparation of compounds within the scope of Formula I of the instant invention is depicted in Scheme 5:

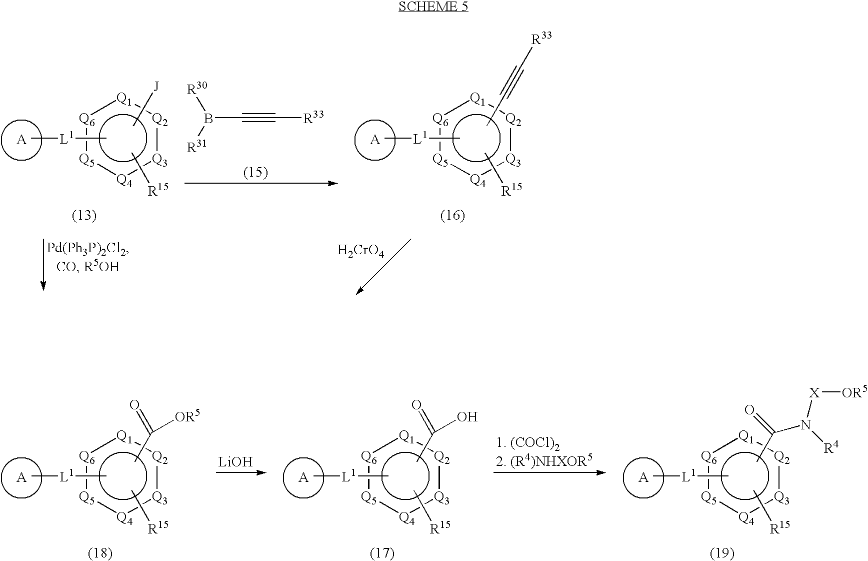

According to this scheme, reaction of intermediate 13 with the organoboron derivative 15 (R³⁰ and R³¹ are independently chosen from lower alkoxy, lower alkyl, cycloalkyl; or R³⁰ and R³¹, together with the atoms to which they are attached, may be joined to form an optionally substituted heterocycloalkyl or optionally substituted cycloalkyl moiety; R³³=hydrogen, lower alkyl, aryl) provides the alkyne intermediate 16. The reaction is conducted in the presence of a suitable catalyst (e.g., Pd(Ph₃)₄, or the like), a suitable base (e.g., Na₂CO₃, or the like) and a suitable solvent (e.g., water, ethanol, DME or the like). The reaction is carried out in the temperature range of about 80° to about 200° C., optionally in a microwave reactor. Oxidation of alkyne 16 with a suitable reagent (e.g., chromic acid, ozone/chromic acid, ozone/pyridinium dichromate, or the like) in a suitable solvent (e.g. acetone, aqueous acetone, acetic acid, ethyl acetate, or the like) affords the carboxylic acid PPAR modulator 17 (Formula I, R¹⁶=R¹⁸=—XC(O)OR⁵; R⁵ as defined herein).

An alternate route to carboxylic acid PPAR modulator 17 commences from via catalytic alkoxycarbonylation of intermediate 13 with carbon monoxide and an alcohol R⁵OH (e.g. ethanol, methanol, n-butanol and the like). The reaction is conducted in the presence of a suitable catalyst (e.g., Pd(Ph₃)₂Cl₂, Pd(OAc)₂, or the like) in the presence of a suitable base (e.g. tri-n-butylamine, diethylamine, sodium acetate, or the like). The reaction is carried out in the temperature range of about 70° to about 200° C., optionally in a sealed pressure bottle, to afford ester PPAR modulator 18 (Formula I, R¹⁶=R¹⁸=—XC(O)OR⁵; R⁵ as defined herein). Hydrolysis of ester 18 with a base (e.g. lithium hydroxide and the like) in a suitable solvent (e.g. methanol, ethanol, aqueous ethanol, aqueous tetrahydrofuran, aqueous dioxane or the like) provides acid PPAR modulator 17.

Reaction of 17 or a salt derivative thereof (e.g. sodium, potassium, lithium salts, or the like) with a halogenating agent (e.g. oxalyl chloride, thionyl choride, cyanuric chloride, or the like), in a suitable solvent (e.g. toluene, dichloroethane, dichoromethane, or the like), optionally in the presence of additives (e.g. N,N-dimethylformamide, trialkylamines, or the like) generates an intermediate acid halide, which is then reacted with an amine (e.g. R⁴NH—X—OR⁵, or the like), in the presence of a suitable base (e.g. N,N-diisopropylethylamine, triethylamine, N-methylmorpholine, or the like) in a suitable solvent (e.g. diethyl ether, tetrahydrofuran, acetonitrile, dichloromethane or the like) to yield the amide PPAR modulator 19 (Formula I, R¹⁶=R¹⁸=R⁴NH—X—OR⁵, as defined herein).

Another route for preparation of compounds within the scope of Formula I of the instant invention is depicted in Scheme 6:

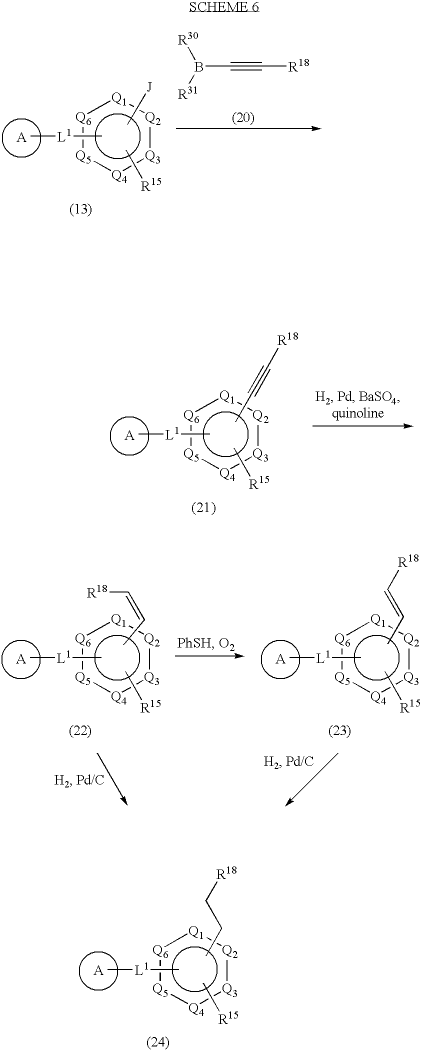

According to this scheme, reaction of intermediate 13 with the organoboron derivative 20 (R³⁰ and R³¹ are independently chosen from lower alkoxy, lower alkyl, cycloalkyl; or R³⁰ and R³¹, together with the atoms to which they are attached, may be joined to form an optionally substituted heterocycloalkyl or optionally substituted cycloalkyl moiety; R¹⁸ as defined herein) provides the alkyne PPAR modulator 21 (Formula I, R¹⁶=—YR¹⁸, Y=lower alkynyl). The reaction is conducted in the presence of a suitable catalyst (e.g., Pd(Ph₃)₄, or the like), a suitable base (e.g., Na₂CO₃, or the like) and a suitable solvent (e.g., water, ethanol, DME or the like). The reaction is carried out in the temperature range of about 80° to about 200° C., optionally in a microwave reactor. Partial hydrogenation of 21 (e.g. hydrogen, diimide or the like), in the presence of a suitable catalyst (e.g. palladium on barium sulfate/quinoline, palladium on calcium carbonate/lead oxide, or the like), in a suitable solvent (e.g. ethanol, methanol, ethyl acetate, tetrahydrofuran, or the like) affords the (Z)-olefin PPAR modulator 22 (Formula I, R¹⁶=—YR¹⁸, Y=lower alkenyl). Isomerization of (Z)-olefin 22 (e.g. thiophenol, oxygen, 70°-150° C., or the like), either neat or optionally in a suitable solvent (e.g. toluene, xylene, 1,2-dichlorobenzene, or the like), yields the (E)-olefin PPAR modulator 23 (Formula I, R¹⁶=—YR¹⁸, Y=lower alkenyl). Further hydrogenation of 22 or 23 in the presence of a suitable catalyst (e.g. palladium on charcoal, palladium hydroxide, platinum oxide, or the like) affords the alkyl-containing PPAR modulator 24 (Formula I, R¹⁶=—YR¹⁸, Y=lower alkyl).

Another route for preparation of compounds within the scope of Formula I of the invention is depicted in Scheme 7:

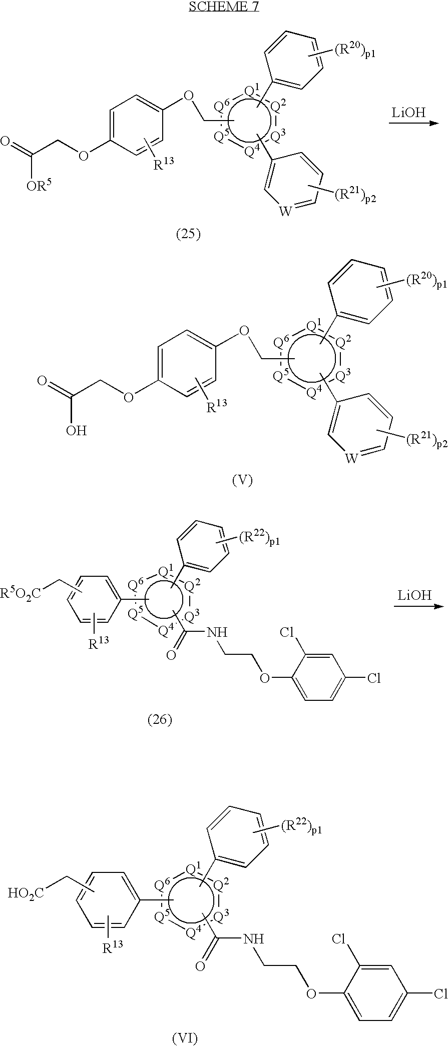

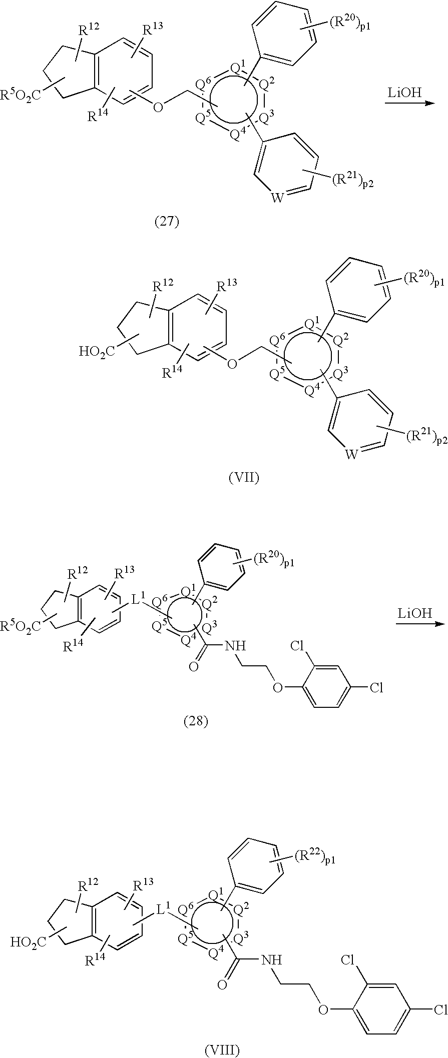

According to this scheme, hydrolysis of ester PPAR modulators 25-28 (e.g. lithium hydroxide, potassium trimethylsilanoate, lithium bromide, lithium thioethoxide, and the like) in a suitable solvent (e.g. methanol, ethanol, tert-butanol, aqueous ethanol, aqueous tetrahydrofuran, aqueous dioxane, N,N-dimethylformamide, N,N-dimethylacetamide, pyridine, lutidine, collidine, or the like) provides acid PPAR modulators (V)-(VIII), respectively.

The following compounds can generally be made using both literature methods and those methods described above. It is expected that these compounds, when made, will have activity as modulators PPAR. The compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36.
CC(C)Oc1ccc(cc1)c2sc(COc3ccc(C)c(OCC(═O)O)c3)cc2c4ccc(OC(F)(F)F)nc4
CC(C)Oc1ccc(cc1)c2cc(COc3ccc(C)c(OCC(═O)O)c3)sc2c4cccnc4
Cc1ccc(OCc2cc(c3ccc(cc3)C(F)(F)F)c(s2)c4ccc(nc4)C(F)(F)F)cc1OCC(═O)O
CSc1ccc(cc1)c2cc(COc3ccc(C)c(OCC(═O)O)c3)sc2c4ccc(cc4)N5CCOCC5
Cc1ccc(OCc2cc(c3cncc(OC(F)(F)F)c3)c(s2)c4ccc(cc4)N5CCOCC5)cc1OCC(═O)O
Cc1ccc(OCc2cc(c3cccc(OC(F)(F)F)c3)c(s2)c4ccc(nc4)N5CCOCC5)cc1OCC(═O)O
COc1ccc(cc1F)c2cc(COc3ccc(C)c(OCC(═O)O)c3)sc2c4ccc(OC(F)(F)F)nc4
COc1ncc(cc1F)c2sc(COc3ccc(C)c(OCC(═O)O)c3)cc2c4cccnc4
COc1cncc(c1)c2cc(COc3ccc(C)c(OCC(═O)O)c3)sc2c4ccc(cc4)C(F)(F)F
COc1ccccc1c2sc(COc3ccc(C)c(OCC(═O)O)c3)cc2c4ccc(SC)nc4
COc1ccncc1c2sc(COc3ccc(C)c(OCC(═O)O)c3)cc2c4ccc(nc4)C(F)(F)F
COc1ccncc1c2cc(COc3ccc(C)c(OCC(═O)O)c3)sc2c4cncc(OC(F)(F)F)c4
CC(C)Oc1ccc(cc1)c2cc(COc3ccc(CC(═O)O)cc3)sc2c4ccc(nc4)c5ccccc5
OC(═O)Cc1ccc(OCc2cc(c3ccc(cc3)C(F)(F)F)c(s2)c4ccc(OC(F)(F)F)nc4)cc1
OC(═O)Cc1ccc(OCc2cc(c3cccnc3)c(s2)c4ccc(cc4)C(F)(F)F)cc1
OC(═O)Cc1ccc(OCc2cc(c3ccc(nc3)c4ccccc4)c(s2)c5ccc(cc5)N6CCOCC6)cc1
OC(═O)Cc1ccc(OCc2cc(c3cccnc3)c(s2)c4ccc(nc4)N5CCOCC5)cc1
COc1ccc(cc1F)c2sc(COc3ccc(CC(═O)O)cc3)cc2c4ccc(OC(F)(F)F)nc4
COc1ccc(cc1F)c2cc(COc3ccc(CC(═O)O)cc3)sc2c4cccnc4
COc1ncc(cc1F)c2sc(COc3ccc(CC(═O)O)cc3)cc2c4ccc(nc4)C(F)(F)F
COc1ccccc1c2cc(COc3ccc(CC(═O)O)cc3)sc2c4cccc(OC(F)(F)F)c4
COc1ccncc1c2sc(COc3ccc(CC(═O)O)cc3)cc2c4ccc(nc4)c5ccccc5
CSc1ccc(cc1)c2sc(COc3cccc(CC(═O)O)c3)cc2c4ccc(OC(C)C)nc4
OC(═O)Cc1cccc(OCc2cc(c3cncc(OC(F)(F)F)c3)c(s2)c4ccc(nc4)c5ccccc5)c1
OC(═O)Cc1cccc(OCc2cc(c3cccc(OC(F)(F)F)c3)c(s2)c4ccc(cc4)N5CCOCC5)c1
CSc1ccc(cn1)c2sc(COc3cccc(CC(═O)O)c3)cc2c4ccc(cc4)N5CCOCC5
COc1ccccc1c2sc(COc3cccc(CC(═O)O)c3)cc2c4ccc(cc4)C(F)(F)F
COc1ccncc1c2cc(COc3cccc(CC(═O)O)c3)sc2c4ccc(nc4)C(F)(F)F
Cc1ccc(OCc2oc(c3ccc(cc3)c4ccccc4)c(c2)c5ccc(OC(F)(F)F)cc5)cc1OCC(═O)O
COc1ccc(cc1F)c2oc(COc3ccc(C)c(OCC(═O)O)c3)cc2c4ccc(nc4)c5ccccc5
CC(C)Oc1ccc(cn1)c2cc(COc3ccc(CC(═O)O)cc3)oc2c4cncc(OC(F)(F)F)c4
OC(═O)Cc1ccc(OCc2oc(c3ccc(nc3)C(F)(F)F)c(c2)c4cccc(OC(F)(F)F)c4)cc1
OC(═O)Cc1ccc(OCc2oc(c3ccc(cc3)c4ccccc4)c(c2)c5ccc(cc5)c6ccccc6)cc1
COc1ccncc1c2oc(COc3ccc(CC(═O)O)cc3)cc2c4cncc(OC(F)(F)F)c4
CC(C)Oc1ccc(cc1)c2oc(COc3cccc(CC(═O)O)c3)cc2c4ccc(nc4)C(F)(F)F
CC(C)Oc1ccc(cc1)c2cc(COc3ccc(C)c(OCC(═O)O)c3)[nH]c2c4ccc(cc4)C(F)(F)F
CC(C)Oc1ccc(cn1)c2cc(COc3ccc(C)c(OCC(═O)O)c3)[nH]c2c4ccc(nc4)C(F)(F)F
COc1ccc(cc1)c2[nH]c(COc3ccc(C)c(OCC(═O)O)c3)cc2c4cccnc4
COc1ccc(cn1)c2cc(COc3ccc(C)c(OCC(═O)O)c3)[nH]c2c4cncc(OC(F)(F)F)c4
Cc1ccc(OCc2cc(c3ccc(OC(F)(F)F)cc3)c([nH]2)c4ccc(nc4)c5cccc5)cc1OCC(═O)O
Cc1ccc(OCc2cc(c3ccc(cc3)N4CCOCC4)c([nH]2)c5ccc(nc5)C(F)(F)F)cc1OCC(═O)O
Cc1ccc(OCc2cc(c3ccc(nc3)N4CCOCC4)c([nH]2)c5ccc(cc5)C(F)(F)F)cc1OCC(═O)O
COc1ccc(cc1F)c2cc(COc3ccc(C)c(OCC(═O)O)c3)[nH]c2c4cccc(OC(F)(F)F)c4
COc1ncc(cc1F)c2[nH]c(COc3ccc(C)c(OCC(═O)O)c3)cc2c4ccc(cc4)C(F)(F)F
COc1cncc(c1)c2cc(COc3ccc(C)c(OCC(═O)O)c3)[nH]c2c4ccc(nc4)C(F)(F)F
CC(C)Oc1ccc(cc1)c2cn(COc3ccc(C)c(OCC(═O)O)c3)cc2c4ccc(OC(F)(F)F)cc4
CC(C)Oc1ccc(cn1)c2cn(COc3ccc(C)c(OCC(═O)O)c3)cc2c4cccnc4
COc1ccc(cc1)c2cn(COc3ccc(C)c(OCC(═O)O)c3)cc2c4ccc(nc4)c5ccccc5
COc1ccc(cn1)c2cn(COc3ccc(C)c(OCC(═O)O)c3)cc2c4ccc(nc4)C(F)(F)F
Cc1ccc(OCn2cc(c3ccc(nc3)C(F)(F)F)c(c2)c4cncc(OC(F)(F)F)c4)cc1OCC(═O)O
Cc1ccc(OCn2cc(c3ccc(cc3)N4CCOCC4)c(c2)c5ccc(cc5)C(F)(F)F)cc1OCC(═O)O
CSc1ccc(cn1)c2cn(COc3ccc(C)c(OCC(═O)O)c3)cc2c4ccc(cc4)N5CCOCC5
Cc1ccc(OCn2cc(c3ccc(nc3)N4CCOCC4)c(c2)c5cncc(OC(F)(F)F)c5)cc1OCC(═O)O
COc1ccc(cc1F)c2cn(COc3ccc(C)c(OCC(═O)O)c3)cc2c4ccc(SC)cc4
COc1ncc(cc1F)c2cn(COc3ccc(C)c(OCC(═O)O)c3)cc2c4cccc(OC(F)(F)F)c4
COc1cncc(c1)c2cn(COc3ccc(C)c(OCC(═O)O)c3)cc2c4cccnc4
COc1ccccc1c2cn(COc3ccc(C)c(OCC(═O)O)c3)cc2c4ccc(OC(F)(F)F)nc4
COc1ccncc1c2cn(COc3ccc(C)c(OCC(═O)O)c3)cc2c4ccc(cc4)C(F)(F)F
CSc1ccc(cc1)n2cc(COc3ccc(C)c(OCC(═O)O)c3)cc2c4ccc(OC(C)C)cc4
CC(C)Oc1ccc(cn1)c2cc(COc3ccc(C)c(OCC(═O)O)c3)cn2c4cncc(OC(F)(F)F)c4
COc1ccc(cn1)n2cc(COc3ccc(C)c(OCC(═O)O)c3)cc2c4ccc(cc4)C(F)(F)F
Cc1ccc(OCc2cc(c3ccc(nc3)c4ccccc4)n(c2)c5ccc(OC(F)(F)F)cc5)cc1OCC(═O)O
Cc1ccc(OCc2cc(c3ccc(nc3)C(F)(F)F)n(c2)c4cccnc4)cc1OCC(═O)O
Cc1ccc(OCc2cc(c3ccc(cc3)N4CCOCC4)n(c2)c5ccc(OC(F)(F)F)nc5)cc1OCC(═O)O
Cc1ccc(OCc2cc(c3ccc(cc3)N4CCOCC4)n(c2)c5ccc(cc5)C(F)(F)F)cc1OCC(═O)O
Cc1ccc(OCc2cc(c3cncc(OC(F)(F)F)c3)n(c2)c4ccc(cc4)N5CCOCC5)cc1OCC(═O)O
Cc1ccc(OCc2cc(c3ccc(nc3)N4CCOCC4)n(c2)c5ccc(cc5)c6ccccc6)cc1OCC(═O)O
COc1ccc(cc1F)n2cc(COc3ccc(C)c(OCC(═O)O)c3)cc2c4ccc(OC(F)(F)F)nc4
COc1ncc(cc1F)c2cc(COc3ccc(C)c(OCC(═O)O)c3)cn2c4ccc(SC)cc4
COc1cncc(c1)n2cc(COc3ccc(C)c(OCC(═O)O)c3)cc2c4ccc(cc4)c5ccccc5
COc1ccccc1c2cc(COc3ccc(C)c(OCC(═O)O)c3)cn2c4cccc(OC(F)(F)F)c4
COc1ccncc1c2cc(COc3ccc(C)c(OCC(═O)O)c3)cn2c4ccc(cc4)C(F)(F)F
COc1ccncc1n2cc(COc3ccc(C)c(OCC(═O)O)c3)cc2c4cncc(OC(F)(F)F)c4
CC(C)Oc1ccc(cc1)c2cc(COc3ccc(CC(═O)O)cc3)[nH]c2c4cccc(OC(F)(F)F)c4
CC(C)Oc1ccc(cn1)c2cc(COc3ccc(CC(═O)O)cc3)[nH]c2c4ccc(cc4)C(F)(F)F
COc1ccc(cc1)c2cc(COc3ccc(CC(═O)O)cc3)[nH]c2c4ccc(SC)nc4
OC(═O)Cc1ccc(OCc2cc(c3ccc(cc3)C(F)(F)F)c([nH]2)c4ccc(nc4)c5ccccc5)cc1
OC(═O)Cc1ccc(OCc2cc(c3ccc(nc3)C(F)(F)F)c([nH]2)c4cccnc4)cc1
CSc1ccc(cc1)c2[nH]c(COc3ccc(CC(═O)O)cc3)cc2c4ccc(cc4)N5CCOCC5
OC(═O)Cc1ccc(OCc2cc(c3cccc(OC(F)(F)F)c3)c([nH]2)c4ccc(nc4)N5CCOCC5)cc1
COc1ccc(cc1F)c2[nH]c(COc3ccc(CC(═O)O)cc3)cc2c4cncc(OC(F)(F)F)c4
CC(C)Oc1ccc(cc1)c2cn(COc3ccc(CC(═O)O)cc3)cc2c4ccc(OC(F)(F)F)nc4
CC(C)Oc1ccc(cc1)c2cn(COc3ccc(CC(═O)O)cc3)cc2c4ccc(cc4)c5ccccc5
COc1ccc(cc1)c2cn(COc3ccc(CC(═O)O)cc3)cc2c4ccc(cc4)C(F)(F)F
COc1ccc(cc1)c2cn(COc3ccc(CC(═O)O)cc3)cc2c4cncc(OC(F)(F)F)c4
OC(═O)Cc1ccc(OCn2cc(c3ccc(nc3)c4ccccc4)c(c2)c5ccc(nc5)C(F)(F)F)cc1
OC(═O)Cc1ccc(OCn2cc(c3ccc(cc3)N4CCOCC4)c(c2)c5cccnc5)cc1
OC(═O)Cc1ccc(OCn2cc(c3ccc(cc3)N4CCOCC4)c(c2)c5cccc(OC(F)(F)F)c5)cc1
OC(═O)Cc1ccc(OCn2cc(c3ccc(nc3)N4CCOCC4)c(c2)c5ccc(nc5)C(F)(F)F)cc1
COc1ccc(cc1F)c2cn(COc3ccc(CC(═O)O)cc3)cc2c4ccc(nc4)C(F)(F)F
COc1ccc(cc1F)c2cn(COc3ccc(CC(═O)O)cc3)cc2c4cccc(OC(F)(F)F)c4
COc1ncc(cc1F)c2cn(COc3ccc(CC(═O)O)cc3)cc2c4cccnc4
COc1ccccc1c2cn(COc3ccc(CC(═O)O)cc3)cc2c4ccc(SC)nc4
COc1ccncc1c2cn(COc3ccc(CC(═O)O)cc3)cc2c4ccccc4
CC(C)Oc1ccc(cc1)n2cc(COc3ccc(CC(═O)O)cc3)cc2c4cccnc4
CC(C)Oc1ccc(cn1)n2cc(COc3ccc(CC(═O)O)cc3)cc2c4ccc(cc4)C(F)(F)F
COc1ccc(cc1)c2cc(COc3ccc(CC(═O)O)cc3)cn2c4cccc(OC(F)(F)F)c4
COc1ccc(cn1)c2cc(COc3ccc(CC(═O)O)cc3)cn2c4ccc(nc4)c5ccccc5
OC(═O)Cc1ccc(OCc2cc(c3ccc(nc3)C(F)(F)F)n(c2)c4cncc(OC(F)(F)F)c4)cc1
OC(═O)Cc1ccc(OCc2cc(c3cccc(OC(F)(F)F)c3)n(c2)c4ccc(nc4)c5ccccc5)cc1
OC(═O)Cc1ccc(OCc2cc(c3ccc(cc3)N4CCOCC4)n(c2)c5cccnc5)cc1
OC(═O)Cc1ccc(OCc2cc(c3ccc(nc3)C(F)(F)F)n(c2)c4ccc(cc4)N5CCOCC5)cc1
OC(═O)Cc1ccc(OCc2cc(c3cncc(OC(F)(F)F)c3)n(c2)c4ccc(nc4)N5CCOCC5)cc1
COc1ccc(cc1F)c2cc(COc3ccc(CC(═O)O)cc3)cn2c4ccc(nc4)C(F)(F)F
COc1ccc(cc1F)n2cc(COc3ccc(CC(═O)O)cc3)cc2c4cncc(OC(F)(F)F)c4
COc1ncc(cc1F)c2cc(COc3ccc(CC(═O)O)cc3)cn2c4ccc(cc4)C(F)(F)F
COc1ncc(cc1F)n2cc(COc3ccc(CC(═O)O)cc3)cc2c4ccc(OC(F)(F)F)cc4
COc1ccncc1c2cc(COc3ccc(CC(═O)O)cc3)cn2c4cccnc4
COc1ccncc1n2cc(COc3ccc(CC(═O)O)cc3)cc2c4ccc(cc4)C(F)(F)F
OC(═O)Cc1cccc(OCc2cc(c3ccc(OC(F)(F)F)nc3)c([nH]2)c4cccc(OC(F)(F)F)c4)c1
OC(═O)Cc1cccc(OCc2cc(c3ccc(nc3)C(F)(F)F)c([nH]2)c4ccc(cc4)N5CCOCC5)c1
COc1ccc(cc1F)c2[nH]c(COc3cccc(CC(═O)O)c3)cc2c4ccc(cc4)C(F)(F)F
COc1ncc(cc1F)c2cc(COc3cccc(CC(═O)O)c3)[nH]c2c4ccc(nc4)c5ccccc5
COc1ccncc1c2cc(COc3cccc(CC(═O)O)c3)[nH]c2c4ccc(SC)cc4
CC(C)Oc1ccc(cn1)c2cn(COc3cccc(CC(═O)O)c3)cc2c4ccc(nc4)C(F)(F)F
OC(═O)Cc1cccc(OCn2cc(c3ccc(cc3)C(F)(F)F)c(c2)c4cccnc4)c1
OC(═O)Cc1cccc(OCn2cc(c3ccc(cc3)N4CCOCC4)c(c2)c5ccc(OC(F)(F)F)nc5)c1
OC(═O)Cc1cccc(OCn2cc(c3ccc(nc3)N4CCOCC4)c(c2)c5ccc(nc5)c6ccccc6)c1
COc1ccc(cc1F)c2cn(COc3cccc(CC(═O)O)c3)cc2c4ccc(nc4)c5ccccc5
COc1ncc(cc1F)c2cn(COc3cccc(CC(═O)O)c3)cc2c4cncc(OC(F)(F)F)c4
CC(C)Oc1ccc(cc1)c2cc(COc3cccc(CC(═O)O)c3)cn2c4ccc(cc4)C(F)(F)F
CC(C)Oc1ccc(cc1)c2cc(COc3cccc(CC(═O)O)c3)cn2c4cncc(OC(F)(F)F)c4
CC(C)Oc1ccc(cn1)c2cc(COc3cccc(CC(═O)O)c3)cn2c4cccc(OC(F)(F)F)c4
OC(═O)Cc1cccc(OCc2cc(c3ccc(nc3)N4CCOCC4)n(c2)c5ccc(cc5)C(F)(F)F)c1
COc1ccc(cc1F)n2cc(COc3cccc(CC(═O)O)c3)cc2c4ccc(nc4)c5ccccc5
COc1cncc(c1)n2cc(COc3cccc(CC(═O)O)c3)cc2c4ccc(OC(F)(F)F)nc4
CC(C)Oc1ccc(cc1)c2nn(COc3ccc(C)c(OCC(═O)O)c3)cc2c4ccc(nc4)C(F)(F)F
CC(C)Oc1ccc(cn1)c2nn(COc3ccc(C)c(OCC(═O)O)c3)cc2c4cccc(OC(F)(F)F)c4
COc1ccc(cc1)c2nn(COc3ccc(C)c(OCC(═O)O)c3)cc2c4cncc(OC(F)(F)F)c4
COc1ccc(cc1)c2cn(COc3ccc(C)c(OCC(═O)O)c3)nc2c4cccnc4
COc1ccc(cn1)c2cn(COc3ccc(C)c(OCC(═O)O)c3)nc2c4ccc(nc4)c5ccccc5
Cc1ccc(OCn2cc(c3ccc(cc3)C(F)(F)F)c(n2)c4ccc(OC(F)(F)F)cc4)cc1OCC(═O)O
Cc1ccc(OCn2cc(c3ccc(OC(F)(F)F)nc3)c(n2)c4ccc(cc4)N5CCOCC5)cc1OCC(═O)O
Cc1ccc(OCn2cc(c3ccccc3)c(n2)c4ccc(cc4)N5CCOCC5)cc1OCC(═O)O
Cc1ccc(OCn2cc(c3ccc(cc3)C(F)(F)F)c(n2)c4ccc(nc4)N5CCOCC5)cc1OCC(═O)O
COc1ncc(cc1F)c2cn(COc3ccc(C)c(OCC(═O)O)c3)nc2c4ccc(cc4)C(F)(F)F
CC(C)Oc1ccc(cc1)c2cc(COc3ccc(C)c(OCC(═O)O)c3)nn2c4ccc(nc4)c5ccccc5
CC(C)Oc1ccc(cn1)n2nc(COc3ccc(C)c(OCC(═O)O)c3)cc2c4cncc(OC(F)(F)F)c4
COc1ccc(cc1)n2nc(COc3ccc(C)c(OCC(═O)O)c3)cc2c4ccc(nc4)C(F)(F)F
Cc1ccc(OCc2cc(c3cccc(OC(F)(F)F)c3)n(n2)c4ccc(OC(F)(F)F)cc4)cc1OCC(═O)O
CSc1ccc(cn1)n2nc(COc3ccc(C)c(OCC(═O)O)c3)cc2c4ccc(cc4)C(F)(F)F
Cc1ccc(OCc2cc(c3ccc(OC(F)(F)F)nc3)n(n2)c4ccc(cc4)N5CCOCC5)cc1OCC(═O)O
CSc1ccc(cc1)c2cc(COc3ccc(C)c(OCC(═O)O)c3)nn2c4ccc(cc4)N5CCOCC5
Cc1ccc(OCc2cc(c3ccc(cc3)c4ccccc4)n(n2)c5ccc(nc5)N6CCOCC6)cc1OCC(═O)O
COc1ccc(cc1F)c2cc(COc3ccc(C)c(OCC(═O)O)c3)nn2c4cncc(OC(F)(F)F)c4
COc1ncc(cc1F)n2nc(COc3ccc(C)c(OCC(═O)O)c3)cc2c4cccnc4
COc1ncc(cc1F)c2cc(COc3ccc(C)c(OCC(═O)O)c3)nn2c4cccc(OC(F)(F)F)c4
COc1ccncc1n2nc(COc3ccc(C)c(OCC(═O)O)c3)cc2c4ccc(cc4)c5ccccc5
COc1ccncc1c2cc(COc3ccc(C)c(OCC(═O)O)c3)nn2c4ccc(OC(F)(F)F)nc4
CC(C)Oc1ccc(cc1)c2nn(COc3ccc(CC(═O)O)cc3)cc2c4ccc(nc4)c5ccccc5
CC(C)Oc1ccc(cc1)c2cn(COc3ccc(CC(═O)O)cc3)nc2c4cncc(OC(F)(F)F)c4
CC(C)Oc1ccc(cn1)c2cn(COc3ccc(CC(═O)O)cc3)nc2c4ccc(cc4)C(F)(F)F
COc1ccc(cc1)c2cn(COc3ccc(CC(═O)O)cc3)nc2c4ccc(OC(F)(F)F)nc4
OC(═O)Cc1ccc(OCn2cc(c3ccc(nc3)C(F)(F)F)c(n2)c4ccc(OC(F)(F)F)cc4)cc1
OC(═O)Cc1ccc(OCn2cc(c3ccc(cc3)C(F)(F)F)c(n2)c4ccc(cc4)c5ccccc5)cc1
OC(═O)Cc1ccc(OCn2cc(c3ccc(OC(F)(F)F)cc3)c(n2)c4ccc(cc4)N5CCOCC5)cc1
OC(═O)Cc1ccc(OCn2cc(c3ccc(cc3)N4CCOCC4)c(n2)c5ccc(nc5)C(F)(F)F)cc1
OC(═O)Cc1ccc(OCn2cc(c3cccnc3)c(n2)c4ccc(nc4)N5CCOCC5)cc1
COc1ccc(cc1F)c2nn(COc3ccc(CC(═O)O)cc3)cc2c4cccnc4
COc1ncc(cc1F)c2cn(COc3ccc(CC(═O)O)cc3)nc2c4cccc(OC(F)(F)F)c4
COc1ccccc1c2nn(COc3ccc(CC(═O)O)cc3)cc2c4cccc(OC(F)(F)F)c4
COc1ccncc1c2nn(COc3ccc(CC(═O)O)cc3)cc2c4cncc(OC(F)(F)F)c4
CC(C)Oc1ccc(cc1)n2nc(COc3ccc(CC(═O)O)cc3)cc2c4ccc(cc4)C(F)(F)F
CC(C)Oc1ccc(cc1)c2cc(COc3ccc(CC(═O)O)cc3)nn2c4cncc(OC(F)(F)F)c4
CC(C)Oc1ccc(cn1)n2nc(COc3ccc(CC(═O)O)cc3)cc2c4cccnc4
COc1ccc(cc1)n2nc(COc3ccc(CC(═O)O)cc3)cc2c4cncc(OC(F)(F)F)c4
COc1ccc(cc1)c2cc(COc3ccc(CC(═O)O)cc3)nn2c4ccc(nc4)c5ccccc5
OC(═O)Cc1ccc(OCc2cc(c3ccc(OC(F)(F)F)nc3)n(n2)c4ccc(cc4)C(F)(F)F)cc1
OC(═O)Cc1ccc(OCc2cc(c3ccc(cc3)N4CCOCC4)n(n2)c5ccc(nc5)C(F)(F)F)cc1
OC(═O)Cc1ccc(OCc2cc(c3ccc(nc3)N4CCOCC4)n(n2)c5ccc(cc5)c6ccccc6)cc1
COc1ccc(cc1F)c2cc(COc3ccc(CC(═O)O)cc3)nn2c4cccc(OC(F)(F)F)c4
COc1ncc(cc1F)n2nc(COc3ccc(CC(═O)O)cc3)cc2c4cccc(OC(F)(F)F)c4
COc1ncc(cc1F)c2cc(COc3ccc(CC(═O)O)cc3)nn2c4ccc(SC)cc4
COc1ccncc1n2nc(COc3ccc(CC(═O)O)cc3)cc2c4ccc(nc4)C(F)(F)F
COc1ccncc1c2cc(COc3ccc(CC(═O)O)cc3)nn2c4cccnc4
CC(C)Oc1ccc(cc1)c2cn(COc3cccc(CC(═O)O)c3)nc2c4ccc(nc4)C(F)(F)F
OC(═O)Cc1cccc(OCn2cc(c3ccc(cc3)c4ccccc4)c(n2)c5cccnc5)c1
OC(═O)Cc1cccc(OCn2cc(c3cccc(OC(F)(F)F)c3)c(n2)c4ccc(nc4)c5ccccc5)c1
OC(═O)Cc1cccc(OCn2cc(c3ccc(nc3)c4ccccc4)c(n2)c5ccc(cc5)N6CCOCC6)c1
CSc1ccc(cn1)c2nn(COc3cccc(CC(═O)O)c3)cc2c4ccc(cc4)N5CCOCC5
COc1cncc(c1)c2nn(COc3cccc(CC(═O)O)c3)cc2c4ccc(SC)nc4
COc1ccncc1c2nn(COc3cccc(CC(═O)O)c3)cc2c4ccc(nc4)C(F)(F)F
OC(═O)Cc1cccc(OCc2cc(c3ccc(cc3)C(F)(F)F)n(n2)c4cccnc4)c1
OC(═O)Cc1cccc(OCc2cc(c3ccc(nc3)C(F)(F)F)n(n2)c4cccc(OC(F)(F)F)c4)c1
OC(═O)Cc1cccc(OCc2cc(c3cccc(OC(F)(F)F)c3)n(n2)c4ccc(cc4)N5CCOCC5)c1
COc1cncc(c1)n2nc(COc3cccc(CC(═O)O)c3)cc2c4ccc(SC)nc4
COc1ccccc1c2cc(COc3cccc(CC(═O)O)c3)nn2c4ccc(cc4)c5ccccc5
CC(C)Oc1ccc(cc1)c2nc(COc3ccc(C)c(OCC(═O)O)c3)[nH]c2c4ccc(OC(F)(F)F)cc4
CC(C)Oc1ccc(cn1)c2nc(COc3ccc(C)c(OCC(═O)O)c3)[nH]c2c4cccnc4
COc1ccc(cc1)c2nc(COc3ccc(C)c(OCC(═O)O)c3)[nH]c2c4ccc(nc4)c5ccccc5
COc1ccc(cn1)c2[nH]c(COc3ccc(C)c(OCC(═O)O)c3)nc2c4ccc(nc4)C(F)(F)F
Cc1ccc(OCc2nc(c3ccc(cc3)C(F)(F)F)c([nH]2)c4cncc(OC(F)(F)F)c4)cc1OCC(═O)O
Cc1ccc(OCc2nc(c3ccc(cc3)c4ccccc4)c([nH]2)c5ccc(cc5)N6CCOCC6)cc1OCC(═O)O
Cc1ccc(OCc2nc(c3ccc(nc3)N4CCOCC4)c([nH]2)c5ccc(OC(F)(F)F)nc5)cc1OCC(═O)O
COc1ccc(cc1F)c2nc(COc3ccc(C)c(OCC(═O)O)c3)[nH]c2c4ccc(cc4)C(F)(F)F
COc1ncc(cc1F)c2nc(COc3ccc(C)c(OCC(═O)O)c3)[nH]c2c4ccc(OC(F)(F)F)cc4
CC(C)Oc1ccc(cc1)c2nc(COc3ccc(C)c(OCC(═O)O)c3)cn2c4ccc(OC(F)(F)F)cc4
CC(C)Oc1ccc(cn1)n2cc(COc3ccc(C)c(OCC(═O)O)c3)nc2c4ccc(nc4)c5ccccc5
COc1ccc(cc1)c2nc(COc3ccc(C)c(OCC(═O)O)c3)cn2c4cccnc4
COc1ccc(cn1)c2nc(COc3ccc(C)c(OCC(═O)O)c3)cn2c4cncc(OC(F)(F)F)c4
Cc1ccc(OCc2cn(c3ccc(cc3)C(F)(F)F)c(n2)c4ccc(nc4)C(F)(F)F)cc1OCC(═O)O
Cc1ccc(OCc2cn(c3cccc(OC(F)(F)F)c3)c(n2)c4ccc(nc4)c5ccccc5)cc1OCC(═O)O
Cc1ccc(OCc2cn(c3cncc(OC(F)(F)F)c3)c(n2)c4ccc(cc4)N5CCOCC5)cc1OCC(═O)O
Cc1ccc(OCc2cn(c3ccc(cc3)N4CCOCC4)c(n2)c5ccc(cc5)c6ccccc6)cc1OCC(═O)O
Cc1ccc(OCc2cn(c3ccc(nc3)N4CCOCC4)c(n2)c5ccc(cc5)C(F)(F)F)cc1OCC(═O)O
COc1ccc(cc1F)n2cc(COc3ccc(C)c(OCC(═O)O)c3)nc2c4cncc(OC(F)(F)F)c4
COc1ncc(cc1F)c2nc(COc3ccc(C)c(OCC(═O)O)c3)cn2c4ccccc4
COc1ccncc1c2nc(COc3ccc(C)c(OCC(═O)O)c3)cn2c4ccc(OC(F)(F)F)nc4
COc1ccncc1n2cc(COc3ccc(C)c(OCC(═O)O)c3)nc2c4ccc(nc4)c5ccccc5
CC(C)Oc1ccc(cc1)c2[nH]c(COc3ccc(CC(═O)O)cc3)nc2c4ccc(OC(F)(F)F)nc4
COc1ccc(cc1)c2nc(COc3ccc(CC(═O)O)cc3)[nH]c2c4ccc(cc4)c5ccccc5
COc1ccc(cc1)c2[nH]c(COc3ccc(CC(═O)O)cc3)nc2c4cncc(OC(F)(F)F)c4
COc1ccc(cn1)c2[nH]c(COc3ccc(CC(═O)O)cc3)nc2c4cccnc4
OC(═O)Cc1ccc(OCc2nc(c3ccc(cc3)C(F)(F)F)c([nH]2)c4cccc(OC(F)(F)F)c4)cc1
OC(═O)Cc1ccc(OCc2nc(c3ccc(nc3)C(F)(F)F)c([nH]2)c4ccc(nc4)c5ccccc5)cc1
OC(═O)Cc1ccc(OCc2nc(c3ccc(cc3)N4CCOCC4)c([nH]2)c5ccc(cc5)C(F)(F)F)cc1
OC(═O)Cc1ccc(OCc2nc(c3ccc(cc3)c4ccccc4)c([nH]2)c5ccc(nc5)N6CCOCC6)cc1
COc1ccc(cc1F)c2[nH]c(COc3ccc(CC(═O)O)cc3)nc2c4ccc(nc4)c5ccccc5
COc1ncc(cc1F)c2nc(COc3ccc(CC(═O)O)cc3)[nH]c2c4ccc(cc4)C(F)(F)F
COc1ccncc1c2[nH]c(COc3ccc(CC(═O)O)cc3)nc2c4ccc(cc4)C(F)(F)F
CC(C)Oc1ccc(cc1)c2nc(COc3ccc(CC(═O)O)cc3)cn2c4ccc(cc4)C(F)(F)F
CC(C)Oc1ccc(cc1)c2nc(COc3ccc(CC(═O)O)cc3)cn2c4ccc(nc4)c5ccccc5
CSc1ccc(cn1)c2nc(COc3ccc(CC(═O)O)cc3)cn2c4ccc(OC(C)C)cc4
CC(C)Oc1ccc(cn1)c2nc(COc3ccc(CC(═O)O)cc3)cn2c4cncc(OC(F)(F)F)c4
COc1ccc(cc1)c2nc(COc3ccc(CC(═O)O)cc3)cn2c4cccc(OC(F)(F)F)c4
OC(═O)Cc1ccc(OCc2cn(c3ccc(OC(F)(F)F)nc3)c(n2)c4ccc(cc4)C(F)(F)F)cc1
OC(═O)Cc1ccc(OCc2cn(c3ccc(cc3)c4ccccc4)c(n2)c5ccc(nc5)C(F)(F)F)cc1
OC(═O)Cc1ccc(OCc2cn(c3ccc(nc3)C(F)(F)F)c(n2)c4cncc(OC(F)(F)F)c4)cc1
OC(═O)Cc1ccc(OCc2cn(c3cncc(OC(F)(F)F)c3)c(n2)c4ccc(nc4)c5ccccc5)cc1
OC(═O)Cc1ccc(OCc2cn(c3cccnc3)c(n2)c4ccc(cc4)N5CCOCC5)cc1
OC(═O)Cc1ccc(OCc2cn(c3ccc(cc3)N4CCOCC4)c(n2)c5ccc(nc5)c6ccccc6)cc1
OC(═O)Cc1ccc(OCc2cn(c3ccc(OC(F)(F)F)cc3)c(n2)c4ccc(nc4)N5CCOCC5)cc1
COc1ccc(cc1F)c2nc(COc3ccc(CC(═O)O)cc3)cn2c4ccc(nc4)C(F)(F)F
COc1ccc(cc1F)n2cc(COc3ccc(CC(═O)O)cc3)nc2c4cccnc4
COc1ncc(cc1F)c2nc(COc3ccc(CC(═O)O)cc3)cn2c4ccc(cc4)C(F)(F)F
COc1ncc(cc1F)n2cc(COc3ccc(CC(═O)O)cc3)nc2c4ccc(cc4)c5ccccc5
COc1cncc(c1)n2cc(COc3ccc(CC(═O)O)cc3)nc2c4ccccc4
COc1ccncc1c2nc(COc3ccc(CC(═O)O)cc3)cn2c4ccc(SC)nc4
COc1ccncc1n2cc(COc3ccc(CC(═O)O)cc3)nc2c4ccc(cc4)C(F)(F)F
CC(C)Oc1ccc(cc1)c2nc(COc3cccc(CC(═O)O)c3)[nH]c2c4cccnc4
CC(C)Oc1ccc(cn1)c2[nH]c(COc3cccc(CC(═O)O)c3)nc2c4ccc(nc4)C(F)(F)F
COc1ncc(cc1F)c2[nH]c(COc3cccc(CC(═O)O)c3)nc2c4cncc(OC(F)(F)F)c4
CC(C)Oc1ccc(cc1)n2cc(COc3cccc(CC(═O)O)c3)nc2c4cncc(OC(F)(F)F)c4
CC(C)Oc1ccc(cn1)n2cc(COc3cccc(CC(═O)O)c3)nc2c4ccc(nc4)C(F)(F)F
COc1ccc(cn1)n2cc(COc3cccc(CC(═O)O)c3)nc2c4ccc(nc4)c5ccccc5
OC(═O)Cc1cccc(OCc2cn(c3ccc(cc3)N4CCOCC4)c(n2)c5ccc(cc5)C(F)(F)F)c1
COc1ccc(cc1F)n2cc(COc3cccc(CC(═O)O)c3)nc2c4ccc(OC(F)(F)F)nc4
COc1ncc(cc1F)c2nc(COc3cccc(CC(═O)O)c3)cn2c4cncc(OC(F)(F)F)c4
CSc1ccc(cc1)c2nn(COc3ccc(C)c(OCC(═O)O)c3)nc2c4ccc(OC(C)C)cc4
CC(C)Oc1ccc(cn1)c2nn(COc3ccc(C)c(OCC(═O)O)c3)nc2c4ccc(cc4)C(F)(F)F
COc1ccc(cn1)c2nn(COc3ccc(C)c(OCC(═O)O)c3)nc2c4cccnc4
Cc1ccc(OCn2nc(c3ccc(OC(F)(F)F)cc3)c(n2)c4ccc(nc4)C(F)(F)F)cc1OCC(═O)O
Cc1ccc(OCn2nc(c3ccc(cc3)c4ccccc4)c(n2)c5ccc(cc5)C(F)(F)F)cc1OCC(═O)O
Cc1ccc(OCn2nc(c3ccc(OC(F)(F)F)cc3)c(n2)c4ccc(cc4)N5CCOCC5)cc1OCC(═O)O
Cc1ccc(OCn2nc(c3ccc(cc3)N4CCOCC4)c(n2)c5ccc(nc5)c6ccccc6)cc1OCC(═O)O
Cc1ccc(OCn2nc(c3ccc(OC(F)(F)F)nc3)c(n2)c4ccc(nc4)N5CCOCC5)cc1OCC(═O)O
COc1ccc(cc1F)c2nn(COc3ccc(C)c(OCC(═O)O)c3)nc2c4cccnc4
COc1ccc(cc1F)c2nn(COc3ccc(C)c(OCC(═O)O)c3)nc2c4cccc(OC(F)(F)F)c4
COc1ncc(cc1F)c2nn(COc3ccc(C)c(OCC(═O)O)c3)nc2c4ccc(OC(F)(F)F)cc4
COc1ccncc1c2nn(COc3ccc(C)c(OCC(═O)O)c3)nc2c4ccc(nc4)c5ccccc5
COc1ccncc1c2nn(COc3ccc(C)c(OCC(═O)O)c3)nc2c4cncc(OC(F)(F)F)c4
CC(C)Oc1ccc(cc1)c2nn(COc3ccc(CC(═O)O)cc3)nc2c4cccnc4
CC(C)Oc1ccc(cn1)c2nn(COc3ccc(CC(═O)O)cc3)nc2c4ccc(nc4)c5ccccc5
COc1ccc(cn1)c2nn(COc3ccc(CC(═O)O)cc3)nc2c4ccc(nc4)C(F)(F)F
OC(═O)Cc1ccc(OCn2nc(c3ccccc3)c(n2)c4ccc(OC(F)(F)F)cc4)cc1
OC(═O)Cc1ccc(OCn2nc(c3ccc(cc3)C(F)(F)F)c(n2)c4ccc(nc4)c5ccccc5)cc1
OC(═O)Cc1ccc(OCn2nc(c3ccc(nc3)C(F)(F)F)c(n2)c4cncc(OC(F)(F)F)c4)cc1
OC(═O)Cc1ccc(OCn2nc(c3ccc(cc3)N4CCOCC4)c(n2)c5ccc(cc5)C(F)(F)F)cc1
OC(═O)Cc1ccc(OCn2nc(c3ccc(cc3)c4ccccc4)c(n2)c5ccc(nc5)N6CCOCC6)cc1
COc1ccc(cc1F)c2nn(COc3ccc(CC(═O)O)cc3)nc2c4cncc(OC(F)(F)F)c4
COc1ncc(cc1F)c2nn(COc3ccc(CC(═O)O)cc3)nc2c4ccc(nc4)c5ccccc5
COc1ccncc1c2nn(COc3ccc(CC(═O)O)cc3)nc2c4ccc(cc4)C(F)(F)F
CSc1ccc(cc1)c2nn(COc3cccc(CC(═O)O)c3)nc2c4ccc(OC(C)C)cc4
CC(C)Oc1ccc(cn1)c2nn(COc3cccc(CC(═O)O)c3)nc2c4cncc(OC(F)(F)F)c4
OC(═O)Cc1cccc(OCn2nc(c3ccc(cc3)N4CCOCC4)c(n2)c5cncc(OC(F)(F)F)c5)c1
COc1ccc(cc1F)c2nn(COc3cccc(CC(═O)O)c3)nc2c4ccc(cc4)C(F)(F)F
COc1cncc(c1)c2nn(COc3cccc(CC(═O)O)c3)nc2c4ccc(nc4)c5ccccc5
COc1ccccc1c2nn(COc3cccc(CC(═O)O)c3)nc2c4cccnc4
COc1ccncc1c2nn(COc3cccc(CC(═O)O)c3)nc2c4ccc(OC(F)(F)F)nc4
CC(C)Oc1ccc(cc1)c2nc(COc3ccc(C)c(OCC(═O)O)c3)nn2c4ccc(cc4)c5ccccc5
CC(C)Oc1ccc(cc1)c2nc(COc3ccc(C)c(OCC(═O)O)c3)nn2c4cncc(OC(F)(F)F)c4
CC(C)Oc1ccc(cn1)c2nc(COc3ccc(C)c(OCC(═O)O)c3)nn2c4cccnc4
COc1ccc(cc1)c2nc(COc3ccc(C)c(OCC(═O)O)c3)nn2c4ccc(OC(F)(F)F)nc4
Cc1ccc(OCc2nc(c3cncc(OC(F)(F)F)c3)n(n2)c4ccc(OC(F)(F)F)cc4)cc1OCC(═O)O
Cc1ccc(OCc2nc(c3ccc(nc3)C(F)(F)F)n(n2)c4ccc(nc4)c5ccccc5)cc1OCC(═O)O
Cc1ccc(OCc2nc(c3ccc(cc3)N4CCOCC4)n(n2)c5cccnc5)cc1OCC(═O)O
Cc1ccc(OCc2nc(c3ccc(nc3)N4CCOCC4)n(n2)c5ccc(cc5)C(F)(F)F)cc1OCC(═O)O
Cc1ccc(OCc2nc(c3ccc(nc3)c4ccccc4)n(n2)c5ccc(nc5)N6CCOCC6)cc1OCC(═O)O
COc1ccc(cc1F)c2nc(COc3ccc(C)c(OCC(═O)O)c3)nn2c4ccc(nc4)c5ccccc5
COc1ccc(cc1F)n2nc(COc3ccc(C)c(OCC(═O)O)c3)nc2c4ccc(cc4)C(F)(F)F
COc1ncc(cc1F)c2nc(COc3ccc(C)c(OCC(═O)O)c3)nn2c4ccccc4
COc1ncc(cc1F)n2nc(COc3ccc(C)c(OCC(═O)O)c3)nc2c4ccc(nc4)C(F)(F)F
COc1cncc(c1)n2nc(COc3ccc(C)c(OCC(═O)O)c3)nc2c4ccc(cc4)c5ccccc5
COc1ccccc1c2nc(COc3ccc(C)c(OCC(═O)O)c3)nn2c4cccc(OC(F)(F)F)c4
COc1ccncc1c2nc(COc3ccc(C)c(OCC(═O)O)c3)nn2c4ccc(SC)cc4
COc1ccncc1n2nc(COc3ccc(C)c(OCC(═O)O)c3)nc2c4ccc(nc4)c5ccccc5
CC(C)Oc1ccc(cc1)n2nc(COc3ccc(CC(═O)O)cc3)nc2c4ccc(nc4)C(F)(F)F
CC(C)Oc1ccc(cc1)n2nc(COc3ccc(CC(═O)O)cc3)nc2c4cccc(OC(F)(F)F)c4
CC(C)Oc1ccc(cn1)c2nc(COc3ccc(CC(═O)O)cc3)nn2c4ccc(nc4)c5ccccc5
COc1ccc(cc1)c2nc(COc3ccc(CC(═O)O)cc3)nn2c4ccc(SC)cc4
COc1ccc(cn1)c2nc(COc3ccc(CC(═O)O)cc3)nn2c4cncc(OC(F)(F)F)c4
OC(═O)Cc1ccc(OCc2nc(c3ccc(cc3)N4CCOCC4)n(n2)c5ccc(nc5)C(F)(F)F)cc1
OC(═O)Cc1ccc(OCc2nc(c3ccc(cc3)N4CCOCC4)n(n2)c5cccc(OC(F)(F)F)c5)cc1
OC(═O)Cc1ccc(OCc2nc(c3ccc(nc3)N4CCOCC4)n(n2)c5cccnc5)cc1
COc1ccc(cc1F)n2nc(COc3ccc(CC(═O)O)cc3)nc2c4ccc(nc4)c5ccccc5
COc1ncc(cc1F)n2nc(COc3ccc(CC(═O)O)cc3)nc2c4ccc(cc4)c5ccccc5
COc1cncc(c1)c2nc(COc3ccc(CC(═O)O)cc3)nn2c4ccc(cc4)C(F)(F)F
COc1ccncc1c2nc(COc3ccc(CC(═O)O)cc3)nn2c4cccc(OC(F)(F)F)c4
COc1ccncc1n2nc(COc3ccc(CC(═O)O)cc3)nc2c4ccc(nc4)C(F)(F)F
CC(C)Oc1ccc(cc1)c2nc(COc3cccc(CC(═O)O)c3)nn2c4cccnc4
OC(═O)Cc1cccc(OCc2nc(c3ccc(cc3)C(F)(F)F)n(n2)c4ccc(OC(F)(F)F)nc4)c1
OC(═O)Cc1cccc(OCc2nc(c3cncc(OC(F)(F)F)c3)n(n2)c4ccc(nc4)C(F)(F)F)c1
OC(═O)Cc1cccc(OCc2nc(c3ccc(nc3)c4ccccc4)n(n2)c5cccc(OC(F)(F)F)c5)c1
OC(═O)Cc1cccc(OCc2nc(c3ccc(nc3)c4ccccc4)n(n2)c5ccc(cc5)N6CCOCC6)c1
COc1ccc(cc1F)c2nc(COc3cccc(CC(═O)O)c3)nn2c4ccc(nc4)C(F)(F)F
COc1ccc(cc1F)n2nc(COc3cccc(CC(═O)O)c3)nc2c4cncc(OC(F)(F)F)c4
COc1ncc(cc1F)c2nc(COc3cccc(CC(═O)O)c3)nn2c4cccc(OC(F)(F)F)c4
COc1cncc(c1)n2nc(COc3cccc(CC(═O)O)c3)nc2c4ccc(cc4)c5ccccc5
COc1ccncc1c2nc(COc3cccc(CC(═O)O)c3)nn2c4ccc(nc4)c5ccccc5
COc1ccncc1n2nc(COc3cccc(CC(═O)O)c3)nc2c4ccc(SC)nc4
CC(C)Oc1ccc(cc1)c2sc(COc3cccc4CC(Cc34)C(═O)O)cc2c5ccc(OC(F)(F)F)nc5
OC(═O)C1CCc2ccc(OCc3cc(c4ccc(nc4)c5ccccc5)c(s3)c6ccc(nc6)C(F)(F)F)cc12
OC(═O)Cn1ccc2cc(OCc3cc(c4ccc(cc4)N5CCOCC5)c(s3)c6cccnc6)ccc12
COc1ccc(cc1F)c2sc(COc3ccc4c(ccn4CC(═O)O)c3)cc2c5ccc(OC(F)(F)F)nc5
COc1ccccc1c2sc(COc3ccc4C(CC(═O)O)CCc4c3)cc2c5ccc(cc5)c6ccccc6
COc1ccncc1c2cc(COc3ccc4CCN(CC(═O)O)c4c3)sc2c5cncc(OC(F)(F)F)c5
COc1ccc(cc1F)c2cc(COc3cccc4c(CC(═O)O)csc34)sc2c5ccc(nc5)C(F)(F)F
OC(═O)CC1Cc2cccc(OCc3cc(c4ccc(cc4)N5CCOCC5)c(s3)c6ccc(cc6)C(F)(F)F)c2O1
COc1ncc(cc1F)c2sc(COc3cccc4CCC(Oc34)C(═O)O)cc2c5cccnc5
OC(═O)Cc1csc2ccc(OCc3cc(c4cncc(OC(F)(F)F)c4)c(s3)c5ccc(nc5)N6CCOCC6)cc12
COc1ccncc1c2sc(COc3ccc4scc(CC(═O)O)c4c3)cc2c5ccc(nc5)c6ccccc6
COc1ccccc1c2cc(COc3ccc4OC(Cc4c3)C(═O)O)sc2c5ccc(nc5)c6ccccc6
OC(═O)CC1CCc2cc(OCc3cc(c4cccc(OC(F)(F)F)c4)c(s3)c5ccc(nc5)C(F)(F)F)ccc2O1
Cc1cc(OCc2cc(c3ccc(nc3)C(F)(F)F)c(s2)c4ccc(nc4)N5CCOCC5)ccc1C═CC(═O)O
COc1ccc(cc1F)c2cc(COc3ccc(C═CC(═O)O)c(C)c3)sc2c4cccc(OC(F)(F)F)c4
Cc1cc(OCc2cc(c3ccc(cc3)C(F)(F)F)c(s2)c4cccnc4)ccc1OC(C)(C)C(═O)O
COc1cncc(c1)c2cc(COc3cccc4CC(CC(═O)O)Cc34)oc2c5ccc(cc5)C(F)(F)F
CSc1ccc(cn1)c2oc(COc3ccc4CC(CC(═O)O)Cc4c3)cc2c5ccc(cc5)N6CCOCC6
OC(═O)C1Cc2cccc(OCn3cc(c4ccc(cc4)c5ccccc5)c(c3)c6ccc(cc6)c7ccccc7)c2C1
COc1ccc(cc1F)c2cc(COc3cccc4CC(Cc34)C(═O)O)cn2c5ccc(nc5)c6ccccc6
COc1ccc(cc1F)c2[nH]c(COc3ccc4CC(Cc4c3)C(═O)O)cc2c5cccnc5
COc1ccc(cn1)c2cn(COc3ccc4CCC(C(═O)O)c4c3)cc2c5cncc(OC(F)(F)F)c5
OC(═O)C1CCc2ccc(OCc3cc(c4ccc(cc4)N5CCOCC5)n(c3)c6ccc(OC(F)(F)F)cc6)cc12
CSc1ccc(cc1)c2cn(COc3ccc4c(ccn4CC(═O)O)c3)cc2c5ccc(OC(C)C)cc5
OC(═O)Cn1ccc2cc(OCc3cc(c4cncc(OC(F)(F)F)c4)n(c3)c5ccc(cc5)C(F)(F)F)ccc12
OC(═O)CC1Cc2ccc(OCn3cc(c4ccc(cc4)N5CCOCC5)c(c3)c6cccnc6)cc2C1
CC(C)Oc1ccc(cn1)c2cc(COc3ccc4CC(CC(═O)O)Cc4c3)cn2c5cncc(OC(F)(F)F)c5
COc1ccc(cc1F)c2cn(COc3ccc4C(CC(═O)O)CCc4c3)cc2c5ccc(cc5)C(F)(F)F
CC(C)Oc1ccc(cc1)c2[nH]c(COc3ccc4CCN(CC(═O)O)c4c3)cc2c5ccccc5
OC(═O)CN1CCc2ccc(OCn3cc(c4ccc(nc4)N5CCOCC5)c(c3)c6ccc(nc6)c7ccccc7)cc12
COc1ncc(cc1F)c2cc(COc3ccc4CCN(CC(═O)O)c4c3)cn2c5ccc(nc5)C(F)(F)F
OC(═O)Cc1csc2c(OCn3cc(c4ccc(OC(F)(F)F)cc4)c(c3)c5ccc(cc5)N6CCOCC6)cccc12
OC(═O)Cc1csc2c(OCc3cc(c4ccc(cc4)C(F)(F)F)n(c3)c5ccc(nc5)N6CCOCC6)cccc12
COc1ccncc1c2cn(COc3cccc4CC(Oc34)C(═O)O)cc2c5ccc(nc5)C(F)(F)F
COc1ccccc1c2cc(COc3cccc4CC(CC(═O)O)Oc34)[nH]c2c5ccc(OC(F)(F)F)nc5
COc1ccncc1c2cc(COc3cccc4CC(CC(═O)O)Oc34)cn2c5ccc(SC)cc5
CC(C)Oc1ccc(cc1)c2cn(COc3cccc4CCC(Oc34)C(═O)O)cc2c5cccc(OC(F)(F)F)c5
CC(C)Oc1ccc(cn1)c2cn(COc3ccc4scc(CC(═O)O)c4c3)cc2c5ccc(nc5)C(F)(F)F
CC(C)Oc1ccc(cc1)n2cc(COc3ccc4scc(CC(═O)O)c4c3)cc2c5cccnc5
COc1ncc(cc1F)c2cn(COc3ccc4OC(CC(═O)O)Cc4c3)cc2c5ccccc5
COc1cncc(c1)c2cn(COc3ccc(C═CC(═O)O)c(C)c3)cc2c4ccc(nc4)c5ccccc5
CC(C)Oc1ccc(cc1)n2cc(COc3ccc(C═CC(═O)O)c(C)c3)cc2c4ccc(cc4)C(F)(F)F
Cc1cc(OCc2cc(c3ccc(nc3)c4ccccc4)c([nH]2)c5ccc(cc5)N6CCOCC6)ccc1OC(C)(C)C(═O)O
COc1ccncc1c2cn(COc3ccc(OC(C)(C)C(═O)O)c(C)c3)cc2c4ccc(OC(F)(F)F)cc4
Cc1cc(OCc2cc(c3cccc(OC(F)(F)F)c3)n(c2)c4ccc(nc4)C(F)(F)F)ccc1OC(C)(C)C(═O)O
COc1ncc(cc1F)c2nn(COc3cccc4CC(Cc34)C(═O)O)cc2c5ccc(nc5)C(F)(F)F
OC(═O)C1Cc2ccc(OCc3cc(c4ccc(cc4)C(F)(F)F)n(n3)c5ccc(OC(F)(F)F)cc5)cc2C1
CC(C)Oc1ccc(cc1)c2cn(COc3ccc4CCC(C(═O)O)c4c3)nc2c5ccccc5
COc1ccncc1c2cc(COc3ccc4CCC(C(═O)O)c4c3)nn2c5ccc(SC)nc5
OC(═O)Cn1ccc2cc(OCn3cc(c4ccc(nc4)N5CCOCC5)c(n3)c6ccc(cc6)C(F)(F)F)ccc12
CC(C)Oc1ccc(cc1)n2nc(COc3ccc4c(ccn4CC(═O)O)c3)cc2c5ccc(nc5)C(F)(F)F
COc1ccc(cc1F)c2cn(COc3ccc4CCN(CC(═O)O)c4c3)nc2c5ccc(OC(F)(F)F)cc5
OC(═O)CN1CCc2ccc(OCc3cc(c4cccc(OC(F)(F)F)c4)n(n3)c5ccc(nc5)N6CCOCC6)cc12
COc1cncc(c1)c2cn(COc3cccc4c(CC(═O)O)csc34)nc2c5ccc(cc5)C(F)(F)F
CC(C)Oc1ccc(cn1)c2cc(COc3cccc4c(CC(═O)O)csc34)nn2c5ccc(nc5)c6ccccc6
CC(C)Oc1ccc(cn1)c2nn(COc3cccc4CC(Oc34)C(═O)O)cc2c5cncc(OC(F)(F)F)c5
COc1ccc(cc1F)c2cc(COc3cccc4CC(Oc34)C(═O)O)nn2c5ccc(nc5)C(F)(F)F
OC(═O)CC1Cc2cc(OCn3cc(c4ccc(nc4)C(F)(F)F)c(n3)c5ccc(cc5)N6CCOCC6)ccc2O1
COc1ccncc1c2cn(COc3ccc4OC(CCc4c3)C(═O)O)nc2c5cccnc5
OC(═O)C1CCc2cc(OCc3cc(c4cccnc4)n(n3)c5ccc(cc5)N6CCOCC6)ccc2O1
Cc1cc(OCn2cc(c3ccc(cc3)C(F)(F)F)c(n2)c4ccc(OC(F)(F)F)nc4)ccc1C═CC(═O)O
Cc1cc(OCc2cc(c3cncc(OC(F)(F)F)c3)n(n2)c4ccc(nc4)C(F)(F)F)ccc1C═CC(═O)O
COc1ccc(cc1F)c2nn(COc3ccc(OC(C)(C)C(═O)O)c(C)c3)cc2c4ccc(SC)nc4
COc1ncc(cc1F)c2cc(COc3ccc(OC(C)(C)C(═O)O)c(C)c3)nn2c4cccc(OC(F)(F)F)c4
OC(═O)C1Cc2cccc(OCc3nc(c4ccc(nc4)N5CCOCC5)c([nH]3)c6cccnc6)c2C1
OC(═O)C1Cc2cccc(OCc3cn(c4ccc(nc4)C(F)(F)F)c(n3)c5ccc(cc5)N6CCOCC6)c2C1
COc1ccc(cc1)c2nc(COc3ccc4CCC(C(═O)O)c4c3)[nH]c2c5cccc(OC(F)(F)F)c5
COc1ccncc1c2nc(COc3ccc4c(ccn4CC(═O)O)c3)[nH]c2c5ccc(SC)cc5
COc1ncc(cc1F)c2nc(COc3ccc4c(ccn4CC(═O)O)c3)cn2c5cccnc5
COc1ncc(cc1F)c2nc(COc3cccc4CC(CC(═O)O)Cc34)cn2c5cccc(OC(F)(F)F)c5
CC(C)Oc1ccc(cc1)n2cc(COc3ccc4C(CC(═O)O)CCc4c3)nc2c5ccc(cc5)C(F)(F)F
COc1ccc(cc1F)c2[nH]c(COc3ccc4CCN(CC(═O)O)c4c3)nc2c5ccc(nc5)c6ccccc6
COc1ccc(cc1F)n2cc(COc3ccc4CCN(CC(═O)O)c4c3)nc2c5ccc(SC)cc5
COc1ccncc1c2nc(COc3cccc4c(CC(═O)O)csc34)[nH]c2c5ccc(OC(F)(F)F)nc5
CC(C)Oc1ccc(cc1)c2nc(COc3cccc4c(CC(═O)O)csc34)cn2c5cncc(OC(F)(F)F)c5
COc1cncc(c1)c2nc(COc3cccc4CC(Oc34)C(═O)O)cn2c5ccc(SC)nc5
OC(═O)C1CCc2cccc(OCc3nc(c4ccc(cc4)C(F)(F)F)c([nH]3)c5ccc(nc5)C(F)(F)F)c2O1
OC(═O)Cc1csc2ccc(OCc3nc(c4ccc(cc4)N5CCOCC5)c([nH]3)c6ccc(cc6)C(F)(F)F)cc12
COc1ncc(cc1F)c2[nH]c(COc3ccc4OC(Cc4c3)C(═O)O)nc2c5cncc(OC(F)(F)F)c5
COc1ccncc1n2cc(COc3ccc4OC(CC(═O)O)Cc4c3)nc2c5ccc(nc5)C(F)(F)F
CSc1ccc(cc1)c2nc(COc3ccc(C═CC(═O)O)c(C)c3)[nH]c2c4ccc(OC(C)C)nc4
Cc1cc(OCc2cn(c3ccc(cc3)c4ccccc4)c(n2)c5cccnc5)ccc1C═CC(═O)O
CC(C)Oc1ccc(cn1)c2nc(COc3ccc(OC(C)(C)C(═O)O)c(C)c3)cn2c4ccc(nc4)c5ccccc5
OC(═O)C1Cc2ccc(OCn3nc(c4ccc(cc4)c5ccccc5)c(n3)c6ccc(nc6)c7ccccc7)cc2C1
COc1ccncc1c2nn(COc3ccc4C(CC(═O)O)CCc4c3)nc2c5ccc(nc5)C(F)(F)F
CC(C)Oc1ccc(cn1)c2nn(COc3ccc4CCN(CC(═O)O)c4c3)nc2c5cccnc5
CSc1ccc(cc1)c2nn(COc3cccc4CCC(Oc34)C(═O)O)nc2c5ccc(cc5)N6CCOCC6
COc1cncc(c1)c2nn(COc3ccc4scc(CC(═O)O)c4c3)nc2c5ccc(SC)nc5
OC(═O)CC1Cc2cc(OCn3nc(c4ccc(OC(F)(F)F)cc4)c(n3)c5ccc(cc5)C(F)(F)F)ccc2O1
CSc1ccc(cc1)c2nn(COc3ccc(C═CC(═O)O)c(C)c3)nc2c4ccc(OC(C)C)cc4
Cc1cc(OCn2nc(c3ccc(nc3)N4CCOCC4)c(n2)c5cncc(OC(F)(F)F)c5)ccc1CCC(═O)O
COc1ccncc1c2nc(COc3cccc4CC(Cc34)C(═O)O)nn2c5ccccc5
OC(═O)C1CCc2ccc(OCc3nc(c4ccc(nc4)N5CCOCC5)n(n3)c6ccc(cc6)C(F)(F)F)cc12
CC(C)Oc1ccc(cn1)c2nc(COc3ccc4c(ccn4CC(═O)O)c3)nn2c5cncc(OC(F)(F)F)c5
COc1ccc(cc1F)c2nc(COc3ccc4C(CC(═O)O)CCc4c3)nn2c5cncc(OC(F)(F)F)c5
CC(C)Oc1ccc(cc1)n2nc(COc3ccc4CCN(CC(═O)O)c4c3)nc2c5ccc(OC(F)(F)F)cc5
CC(C)Oc1ccc(cn1)n2nc(COc3cccc4CC(CC(═O)O)Oc34)nc2c5ccc(nc5)c6ccccc6
COc1cncc(c1)c2nc(COc3ccc4scc(CC(═O)O)c4c3)nn2c5ccc(nc5)C(F)(F)F
COc1ccccc1n2nc(COc3ccc(C═CC(═O)O)c(C)c3)nc2c4ccc(cc4)C(F)(F)F
Cc1cc(OCc2nc(c3ccc(cc3)N4CCOCC4)n(n2)c5cccnc5)ccc1OC(C)(C)C(═O)O
CC(C)Oc1ccc(cc1)c2snc(COc3cccc4CC(Cc34)C(═O)O)c2c5ccc(OC(F)(F)F)nc5
CSc1ccc(cc1)c2nsc(c2COc3ccc4c(ccn4CC(═O)O)c3)c5ccc(cc5)N6CCOCC6
COc1ccc(cc1F)c2nsc(c2COc3cccc4CC(CC(═O)O)Oc34)c5ccc(cc5)C(F)(F)F
COc1ccncc1c2snc(c2COc3ccc(C═CC(═O)O)c(C)c3)c4cncc(OC(F)(F)F)c4
OC(═O)CC1CCc2cc(OCc3scc(c4ccc(cc4)N5CCOCC5)c3c6ccc(cc6)c7ccccc7)ccc12
OC(═O)CN1CCc2ccc(OCc3cc(sc3c4ccc(nc4)C(F)(F)F)c5ccc(nc5)C(F)(F)F)cc12
COc1ncc(cc1F)c2csc(c2COc3cccc4c(CC(═O)O)csc34)c5cccnc5
OC(═O)C1CCc2cccc(OCc3csc(c4ccc(nc4)N5CCOCC5)c3c6ccc(nc6)c7ccccc7)c2O1
OC(═O)Cc1csc2ccc(OCc3sc(cc3c4ccc(cc4)N5CCOCC5)c6ccc(OC(F)(F)F)nc6)cc12
COc1ccncc1c2cc(c(COc3ccc4OC(CCc4c3)C(═O)O)s2)c5ccc(cc5)c6ccccc6
Cc1cc(OCc2csc(c3ccc(cc3)C(F)(F)F)c2c4ccc(OC(F)(F)F)cc4)ccc1OC(C)(C)C(═O)O
CC(C)Oc1ccc(cc1)c2c(COc3ccc4c(ccn4CC(═O)O)c3)occ2c5cncc(OC(F)(F)F)c5
COc1ccccc1c2coc(c2COc3cccc4CC(CC(═O)O)Cc34)c5ccc(cc5)c6ccccc6
Cc1cc(OCc2occ(c3ccc(nc3)C(F)(F)F)c2c4ccc(nc4)c5ccccc5)ccc1C═CC(═O)O
OC(═O)C1CCc2ccc(OCc3c(onc3c4ccc(nc4)c5ccccc5)c6ccc(nc6)c7ccccc7)cc12
COc1ccc(cc1)c2c(COc3ccc(OC(C)(C)C(═O)O)c(C)c3)onc2c4cccnc4
OC(═O)C1Cc2cccc(OCc3cn(cc3c4cncc(OC(F)(F)F)c4)c5ccc(cc5)N6CCOCC6)c2C1
COc1ccc(cc1F)n2c(COc3ccc4CC(Cc4c3)C(═O)O)ccc2c5ccccc5
COc1cncc(c1)c2cc(c3ccc(cc3)C(F)(F)F)n(COc4ccc5CC(Cc5c4)C(═O)O)c2
COc1ncc(cc1F)c2ccn(c2COc3ccc4CCC(C(═O)O)c4c3)c5cncc(OC(F)(F)F)c5
COc1ncc(cc1F)c2c(ccn2COc3ccc4c(ccn4CC(═O)O)c3)c5ccc(nc5)c6ccccc6
CC(C)Oc1ccc(cn1)c2cc(COc3ccc4c(ccn4CC(═O)O)c3)c([nH]2)c5ccc(cc5)C(F)(F)F
CC(C)Oc1ccc(cn1)c2c(COc3ccc4CC(CC(═O)O)Cc4c3)ccn2c5cccnc5
CC(C)Oc1ccc(cc1)c2cc(COc3ccc4C(CC(═O)O)CCc4c3)c([nH]2)c5ccc(OC(F)(F)F)cc5
OC(═O)C1Cc2cccc(OCn3c(ccc3c4cccc(OC(F)(F)F)c4)c5ccc(cc5)N6CCOCC6)c2O1
COc1ccc(cc1F)c2c(COc3ccc4OC(Cc4c3)C(═O)O)[nH]cc2c5ccc(OC(F)(F)F)cc5
COc1ncc(cc1F)n2cc(COc3ccc4OC(Cc4c3)C(═O)O)c(c2)c5ccc(cc5)C(F)(F)F
OC(═O)CC1CCc2cccc(OCc3ccc(c4cncc(OC(F)(F)F)c4)n3c5ccc(nc5)C(F)(F)F)c2O1
CC(C)Oc1ccc(cc1)c2c(ccn2COc3ccc4OC(CC(═O)O)CCc4c3)c5ccccc5
Cc1cc(OCn2ccc(c3ccc(cc3)N4CCOCC4)c2c5cccnc5)ccc1C═CC(═O)O
COc1cccc(c1)n2cc(COc3ccc(C═CC(═O)O)c(C)c3)c(c2)c4ccc(SC)cc4
Cc1cc(OCc2cc(cn2c3ccc(nc3)N4CCOCC4)c5ccc(nc5)C(F)(F)F)ccc1OC(C)(C)C(═O)O
CC(C)Oc1ccc(cn1)c2ccc(c3ccc(nc3)c4ccccc4)n2COc5ccc(OC(C)(C)C(═O)O)c(C)c5
COc1ccncc1n2ncc(c2COc3cccc4CC(Cc34)C(═O)O)c5ccc(cc5)C(F)(F)F
COc1ccccc1c2c(cnn2COc3ccc4CCC(C(═O)O)c4c3)c5cccc(OC(F)(F)F)c5
COc1ncc(cc1F)c2cc(c3ccc(nc3)C(F)(F)F)n(COc4cccc5CC(CC(═O)O)Cc45)n2
COc1ccncc1c2nn(cc2COc3ccc4C(CC(═O)O)CCc4c3)c5ccc(nc5)C(F)(F)F
OC(═O)CN1CCc2ccc(OCc3c(cnn3c4ccc(cc4)N5CCOCC5)c6ccc(OC(F)(F)F)cc6)cc12
CC(C)Oc1ccc(cc1)c2cn(nc2COc3cccc4c(CC(═O)O)csc34)c5ccc(nc5)C(F)(F)F
OC(═O)Cc1csc2ccc(OCn3ncc(c4ccc(nc4)c5ccccc5)c3c6ccc(cc6)C(F)(F)F)cc12
COc1ccc(cc1F)c2[nH]nc(c2COc3ccc4scc(CC(═O)O)c4c3)c5ccc(SC)cc5
OC(═O)C1Cc2cc(OCc3cnn(c4cncc(OC(F)(F)F)c4)c3c5ccc(nc5)c6ccccc6)ccc2O1
CSc1ccc(cn1)c2c(cnn2COc3ccc4OC(CC(═O)O)Cc4c3)c5ccc(cc5)N6CCOCC6
OC(═O)C1CCc2cccc(OCc3c(n[nH]c3c4ccc(cc4)N5CCOCC5)c6ccccc6)c2C1
COc1ccc(cc1F)c2cc(c3ccc(cc3)C(F)(F)F)n(COc4ccc(C═CC(═O)O)c(C)c4)N2
Cc1cc(OCc2n[nH]c(c3ccc(cc3)C(F)(F)F)c2c4ccc(cc4)c5ccccc5)ccc1CCC(═O)O
CC(C)Oc1ccc(cc1)c2cc(COc3ccc(OC(C)(C)C(═O)O)c(C)c3)n(n2)c4ccccc4
COc1ncc(cc1F)n2cc(nc2COc3cccc4CC(Cc34)C(═O)O)c5ccc(nc5)c6ccccc6
OC(═O)C1CCc2ccc(OCn3cnc(c4ccc(nc4)N5CCOCC5)c3c6ccc(nc6)C(F)(F)F)cc12
COc1ccncc1c2c(COc3ccc4c(ccn4CC(═O)O)c3)ncn2c5ccc(SC)nc5
OC(═O)CC1Cc2ccc(OCc3ncn(c4ccc(nc4)c5ccccc5)c3c6cccc(OC(F)(F)F)c6)cc2C1
COc1ccncc1c2cn(COc3ccc4CCN(CC(═O)O)c4c3)c(n2)c5ccc(nc5)c6ccccc6
COc1ccc(cc1F)c2cnc(COc3ccc4CCN(CC(═O)O)c4c3)n2c5ccc(OC(F)(F)F)nc5
CC(C)Oc1ccc(cn1)c2nc(cn2COc3cccc4c(CC(═O)O)csc34)c5cccc(OC(F)(F)F)c5
CC(C)Oc1ccc(cc1)c2ncc(COc3cccc4CC(Oc34)C(═O)O)n2c5ccccc5
OC(═O)C1CCc2cccc(OCn3c(cnc3c4ccc(cc4)C(F)(F)F)c5cccnc5)c2O1
COc1ccncc1c2cn(c(COc3ccc4scc(CC(═O)O)c4c3)n2)c5cncc(OC(F)(F)F)c5
COc1cncc(c1)c2ncn(COc3ccc4OC(CC(═O)O)Cc4c3)c2c5cncc(OC(F)(F)F)c5
CC(C)Oc1ccc(cc1)c2[nH]c(nc2COc3ccc4OC(CC(═O)O)Cc4c3)c5ccc(nc5)C(F)(F)F
COc1cncc(c1)c2nc(c(COc3cccc4CCC(Cc34)C(═O)O)[nH]2)c5cccnc5
OC(═O)CC1CCc2cc(OCc3nc(cn3c4ccc(cc4)C(F)(F)F)c5ccc(cc5)N6CCOCC6)ccc2O1
COc1cncc(c1)c2cnc(COc3ccc(CCC(═O)O)c(C)c3)n2c4ccccc4
COc1cncc(c1)n2nc(COc3cccc4c(CC(═O)O)csc34)c(n2)c5ccc(nc5)c6ccccc6
OC(═O)CC1Cc2cccc(OCc3nn(nc3c4ccc(cc4)N5CCOCC5)c6cccnc6)c2O1
OC(═O)C1CCc2ccc(OCc3nn(nc3c4ccc(nc4)C(F)(F)F)c5ccc(cc5)c6ccccc6)cc2C1
OC(═O)C1Cc2cccc(OCn3nnc(c4ccccc4)c3c5ccc(cc5)c6ccccc6)c2C1
CSc1ccc(cc1)n2nnc(c2COc3cccc4CC(CC(═O)O)Cc34)c5ccc(nc5)N6CCOCC6
CC(C)Oc1ccc(cn1)c2c(nnn2COc3ccc4CCN(CC(═O)O)c4c3)c5cncc(OC(F)(F)F)c5
OC(═O)C1CCc2cccc(OCc3c(nnn3c4cccc(OC(F)(F)F)c4)c5ccc(cc5)c6ccccc6)c2O1
COc1ccncc1c2nnn(COc3cccc4CCC(CC(═O)O)Oc34)c2c5ccc(nc5)c6ccccc6
CSc1ccc(cc1)c2c(COc3ccc(C═CC(═O)O)c(C)c3)nnn2c4ccc(OC(C)C)nc4
OC(═O)C1Cc2ccc(OCc3nc(nn3c4ccc(cc4)C(F)(F)F)c5ccc(OC(F)(F)F)cc5)cc2C1
CC(C)Oc1ccc(cc1)c2nc(COc3ccc4c(ccn4CC(═O)O)c3)n(n2)c5ccc(nc5)c6ccccc6
OC(═O)Cn1ccc2cc(OCn3nc(nc3c4ccc(nc4)C(F)(F)F)c5ccc(cc5)C(F)(F)F)ccc12
COc1ccncc1c2nc(c3cccnc3)n(COc4ccc5CC(CC(═O)O)Cc5c4)n2
OC(═O)Cc1csc2c(OCn3nc(nc3c4ccc(cc4)c5ccccc5)c6ccc(nc6)N7CCOCC7)cccc12
COc1ccc(cc1F)c2nc(nn2COc3cccc4CC(Oc34)C(═O)O)c5ccc(OC(F)(F)F)nc5
COc1ccccc1n2nc(nc2COc3cccc4CCC(CC(═O)O)Oc34)c5ccc(SC)nc5
CC(C)Oc1ccc(cc1)c2nc(c3ccc(cc3)c4ccccc4)n(COc5ccc(C═CC(═O)O)c(C)c5)n2
Cc1cc(OCn2nc(nc2c3cncc(OC(F)(F)F)c3)c4ccc(cc4)N5CCOCC5)ccc1OC(C)(C)C(═O)O
OC(═O)C1CCc2ccc(OCc3cncc(c4ccc(OC(F)(F)F)cc4)c3c5cccnc5)cc2C1
CC(C)Oc1ccc(cn1)c2cnc(COc3cccc4CCC(CC(═O)O)Oc34)c(c2)c5ccc(cc5)C(F)(F)F
OC(═O)Cc1csc2c(OCc3cnnc(c4ccc(cc4)N5CCOCC5)c3c6ccc(cc6)C(F)(F)F)cccc12
COc1ccc(cc1F)c2cnnc(COc3ccc(CCC(═O)O)c(C)c3)c2c4ccc(SC)nc4
COc1ccccc1c2nc(COc3ccc4OC(CC(═O)O)Cc4c3)ncc2c5ccc(nc5)c6ccccc6
COc1ncc(cc1F)c2ncc(COc3ccc4OC(CC(═O)O)CCc4c3)c(n2)c5ccc(OC(F)(F)F)nc5
Cc1cc(OCc2cc(nc(n2)c3ccc(nc3)N4CCOCC4)c5cncc(OC(F)(F)F)c5)ccc1CCC(═O)O
COc1ncc(cc1F)c2cc(nc(COc3ccc(OC(C)(C)C(═O)O)c(C)c3)n2)c4cncc(OC(F)(F)F)c4
COc1ccncc1c2nc(COc3ccc4CC(Cc4c3)C(═O)O)nnc2c5ccc(SC)nc5
CSc1ccc(cc1)c2nnc(c(COc3ccc4CC(Cc4c3)C(═O)O)n2)c5ccc(cc5)N6CCOCC6
CC(C)Oc1ccc(cc1)c2nnc(COc3ccc4CCN(CC(═O)O)c4c3)nc2c5ccc(cc5)c6ccccc6
CC(C)Oc1ccc(cc1)c2nnc(c(COc3ccc4scc(CC(═O)O)c4c3)n2)c5cccnc5
OC(═O)C0Cc2cccc(OCc3ccc(c4ccc(OC(F)(F)F)cc4)c(c3)c5ccc(cc5)c6c0ccc6)c2C1
OC(═O)C1Cc2ccc(OCc3cc(ccc3c4ccc(cc4)C(F)(F)F)c5ccc(nc5)N6CCOCC6)cc2C1
COc1ccc(cc1F)c2cc(ccc2COc3ccc4CCC(C(═O)O)c4c3)c5ccc(SC)cc5
COc1cncc(c1)c2ccc(COc3cccc4CC(CC(═O)O)Cc34)c(c2)c5ccc(OC(F)(F)F)nc5
COc1cncc(c1)c2ccc(COc3cccc4CC(Oc34)C(═O)O)cc2c5cccnc5
OC(═O)C1CCc2cccc(OCc3ccc(cc3c4ccc(cc4)c5ccccc5)c6ccccc6)c2O1
CSc1ccc(cc1)c2ccc(COc3ccc(OC(C)(C)C(═O)O)c(C)c3)c(c2)c4ccc(cc4)N5CCOCC5
CC(C)Oc1ccc(cc1)c2nnc(COc3cccc4CC(CC(═O)O)Cc34)n2c5ccc(cc5)c6ccccc6
CC(C)Oc1ccc(cc1)c2snc(COc3ccc(C)c(OCC(═O)O)c3)c2c4ccc(OC(F)(F)F)nc4
Cc1ccc(OCc2c(nsc2c3cncc(OC(F)(F)F)c3)c4ccc(cc4)N5CCOCC5)cc1OCC(═O)O
COc1ccncc1c2snc(COc3ccc(CC(═O)O)cc3)c2c4ccc(nc4)c5ccccc5
CSc1ccc(cn1)c2snc(c2COc3ccc(CC(═O)O)cc3)c4ccc(OC(C)C)nc4
CSc1ccc(cc1)c2nsc(c2COc3cccc(CC(═O)O)c3)c4ccc(cc4)N5CCOCC5
Cc1ccc(OCc2scc(c3ccc(cc3)N4CCOCC4)c2c5ccc(cc5)C(F)(F)F)cc1OCC(═O)O
COc1ccncc1c2sc(cc2COc3ccc(C)c(OCC(═O)O)c3)c4cccnc4
COc1ccc(cc1F)c2cc(sc2COc3ccc(CC(═O)O)cc3)c4cncc(OC(F)(F)F)c4
CC(C)Oc1ccc(cc1)c2scc(COc3ccc(CC(═O)O)cc3)c2c4cccnc4
OC(═O)Cc1ccc(OCc2cc(sc2c3ccc(cc3)C(F)(F)F)c4ccc(cc4)c5ccccc5)cc1
OC(═O)Cc1ccc(OCc2c(csc2c3ccc(nc3)N4CCOCC4)c5ccc(OC(F)(F)F)cc5)cc1
COc1ccc(cn1)c2csc(COc3cccc(CC(═O)O)c3)c2c4ccc(nc4)C(F)(F)F
Cc1ccc(OCc2c(occ2c3ccc(nc3)C(F)(F)F)c4ccc(nc4)c5ccccc5)cc1OCC(═O)O
COc1ccc(cc1F)c2c(COc3ccc(C)c(OCC(═O)O)c3)coc2c4cccc(OC(F)(F)F)c4
CSc1ccc(cc1)c2ccn(c2COc3ccc(C)c(OCC(═O)O)c3)c4ccc(OC(C)C)cc4
COc1ccc(cc1F)c2cc([nH]c2COc3ccc(C)c(OCC(═O)O)c3)c4ccc(SC)nc4
COc1ncc(cc1F)c2cc(COc3ccc(C)c(OCC(═O)O)c3)n(c2)c4cncc(OC(F)(F)F)c4
COc1ccccc1c2ccc(COc3ccc(C)c(OCC(═O)O)c3)n2c4ccc(nc4)c5ccccc5
CC(C)Oc1ccc(cc1)c2ccc(c3ccc(cc3)C(F)(F)F)n2COc4ccc(C)c(OCC(═O)O)c4
CSc1ccc(cn1)c2cc(c3ccc(nc3)N4CCOCC4)n(COc5ccc(C)c(OCC(═O)O)c5)c2
CC(C)Oc1ccc(cc1)c2ccc(COc3ccc(CC(═O)O)cc3)n2c4cccc(OC(F)(F)F)c4
OC(═O)Cc1ccc(OCc2cc(cn2c3ccc(cc3)N4CCOCC4)c5cccnc5)cc1
COc1ccncc1c2ccn(c2COc3ccc(CC(═O)O)cc3)c4ccc(OC(F)(F)F)nc4
CC(C)Oc1ccc(cc1)c2cc(c3ccc(nc3)C(F)(F)F)n(COc4ccc(CC(═O)O)cc4)c2
COc1ncc(cc1F)c2ccc(c3ccc(nc3)c4ccccc4)n2COc5ccc(CC(═O)O)cc5
COc1ccccc1c2ccn(COc3ccc(CC(═O)O)cc3)c2c4cccc(OC(F)(F)F)c4
COc1ncc(cc1F)n2cc(COc3ccc(CC(═O)O)cc3)c(c2)c4ccc(SC)cc4
COc1ccccc1c2cc([nH]c2COc3cccc(CC(═O)O)c3)c4ccccc4
COc1ccncc1n2c(COc3cccc(CC(═O)O)c3)ccc2c4ccc(nc4)C(F)(F)F
CC(C)Oc1ccc(cc1)c2[nH]c(cc2COc3cccc(CC(═O)O)c3)c4cncc(OC(F)(F)F)c4
OC(═O)Cc1cccc(OCc2cn(cc2c3cncc(OC(F)(F)F)c3)c4ccc(cc4)C(F)(F)F)c1
COc1ccc(cc1F)c2[nH]cc(COc3cccc(CC(═O)O)c3)c2c4ccc(OC(F)(F)F)nc4
COc1cncc(c1)n2ccc(COc3cccc(CC(═O)O)c3)c2c4ccc(nc4)c5ccccc5
Cc1ccc(OCc2cc(nn2c3ccc(OC(F)(F)F)cc3)c4ccc(OC(F)(F)F)cc4)cc1OCC(═O)O
COc1ncc(cc1F)c2cc(c3ccc(nc3)C(F)(F)F)n(COc4ccc(C)c(OCC(═O)O)c4)n2
COc1ccccc1c2c(cnn2COc3ccc(C)c(OCC(═O)O)c3)c4ccc(cc4)c5ccccc5
COc1ccc(cc1F)n2cc(c(COc3ccc(C)c(OCC(═O)O)c3)n2)c4cccnc4
COc1ccccc1c2c(COc3ccc(C)c(OCC(═O)O)c3)n[nH]c2c4ccc(cc4)C(F)(F)F
CSc1ccc(cn1)n2ncc(COc3ccc(C)c(OCC(═O)O)c3)c2c4ccc(cc4)C(F)(F)F
CC(C)Oc1ccc(cc1)c2c(COc3ccc(CC(═O)O)cc3)[nH]nc2c4ccc(cc4)C(F)(F)F
COc1ccc(cc1)n2ncc(c2COc3ccc(CC(═O)O)cc3)c4ccc(cc4)c5ccccc5
COc1ncc(cc1F)c2cc(COc3ccc(CC(═O)O)cc3)n(n2)c4ccc(cc4)C(F)(F)F
CSc1ccc(cc1)c2cc(c3ccc(OC(C)C)cc3)n(COc4ccc(CC(═O)O)cc4)n2
OC(═O)Cc1ccc(OCn2ncc(c3ccc(OC(F)(F)F)nc3)c2c4ccc(cc4)C(F)(F)F)cc1
OC(═O)Cc1ccc(OCc2c(n[nH]c2c3ccc(nc3)C(F)(F)F)c4cccc(OC(F)(F)F)c4)cc1
OC(═O)Cc1cccc(OCc2c(cnn2c3ccc(nc3)N4CCOCC4)c5cncc(OC(F)(F)F)c5)c1
OC(═O)Cc1cccc(OCc2nn(cc2c3ccc(nc3)c4ccccc4)c5cccc(OC(F)(F)F)c5)c1
CSc1ccc(cn1)c2c(COc3cccc(CC(═O)O)c3)n[nH]c2c4ccc(nc4)N5CCOCC5
COc1ccncc1n2ncc(COc3cccc(CC(═O)O)c3)c2c4cccc(OC(F)(F)F)c4
Cc1ccc(OCn2c(cnc2c3ccc(OC(F)(F)F)cc3)c4cccnc4)cc1OCC(═O)O
COc1cncc(c1)c2c(ncn2COc3ccc(C)c(OCC(═O)O)c3)c4ccc(nc4)c5ccccc5
CC(C)Oc1ccc(cn1)c2cn(c(COc3ccc(C)c(OCC(═O)O)c3)n2)c4cccnc4
COc1ccncc1n2c(COc3ccc(C)c(OCC(═O)O)c3)ncc2c4cncc(OC(F)(F)F)c4
Cc1ccc(OCc2ncn(c3ccc(nc3)N4CCOCC4)c2c5ccc(cc5)c6ccccc6)cc1OCC(═O)O
CC(C)Oc1ccc(cn1)n2cnc(c2COc3ccc(CC(═O)O)cc3)c4ccc(cc4)C(F)(F)F
OC(═O)Cc1ccc(OCc2[nH]c(nc2c3cccnc3)c4ccc(cc4)C(F)(F)F)cc1
OC(═O)Cc1ccc(OCn2c(cnc2c3ccc(cc3)N4CCOCC4)c5ccc(cc5)C(F)(F)F)cc1
COc1cncc(c1)c2nc(cn2COc3ccc(CC(═O)O)cc3)c4cccnc4
CSc1ccc(cn1)n2c(COc3ccc(CC(═O)O)cc3)ncc2c4ccc(cc4)N5CCOCC5
COc1ccc(cc1F)c2cn(c(COc3ccc(CC(═O)O)cc3)n2)c4ccc(nc4)C(F)(F)F
COc1ccc(cc1F)n2c(COc3cccc(CC(═O)O)c3)cnc2c4ccc(cc4)C(F)(F)F
CC(C)Oc1ccc(cn1)c2c(ncn2COc3cccc(CC(═O)O)c3)c4cccc(OC(F)(F)F)c4
COc1ncc(cc1F)n2cc(nc2COc3cccc(CC(═O)O)c3)c4cncc(OC(F)(F)F)c4
COc1ccccc1c2c(COc3cccc(CC(═O)O)c3)ncn2c4cccnc4
COc1cncc(c1)c2nn(nc2COc3ccc(C)c(OCC(═O)O)c3)c4ccc(nc4)C(F)(F)F
CC(C)Oc1ccc(cc1)c2nn(nc2COc3cccc(CC(═O)O)c3)c4ccc(nc4)c5ccccc5
Cc1ccc(OCn2nnc(c3ccc(cc3)N4CCOCC4)c2c5ccccc5)cc1OCC(═O)O
COc1ccncc1c2nnn(COc3ccc(C)c(OCC(═O)O)c3)c2c4ccc(OC(F)(F)F)nc4
COc1ccncc1n2nnc(c2COc3ccc(C)c(OCC(═O)O)c3)c4ccc(cc4)c5ccccc5
OC(═O)Cc1ccc(OCc2c(nnn2c3cncc(OC(F)(F)F)c3)c4ccc(cc4)C(F)(F)F)cc1
OC(═O)Cc1cccc(OCn2nnc(c3ccc(cc3)c4ccccc4)c2c5cccnc5)c1
COc1cncc(c1)c2c(COc3cccc(CC(═O)O)c3)nnn2c4ccccc4
Cc1ccc(OCc2nc(nn2c3ccc(cc3)c4ccccc4)c5ccc(cc5)c6ccccc6)cc1OCC(═O)O
CC(C)Oc1ccc(cc1)c2nc(c3cccnc3)n(COc4ccc(C)c(OCC(═O)O)c4)n2
CC(C)Oc1ccc(cn1)n2nc(nc2COc3ccc(CC(═O)O)cc3)c4cncc(OC(F)(F)F)c4
COc1ccncc1c2nc(c3ccc(nc3)C(F)(F)F)n(COc4ccc(CC(═O)O)cc4)n2
OC(═O)Cc1cccc(OCc2nc(nn2c3ccc(cc3)N4CCOCC4)c5ccc(nc5)C(F)(F)F)c1
CC(C)Oc1ccc(cc1)c2nc(c3cccc(OC(F)(F)F)c3)n(COc4cccc(CC(═O)O)c4)n2
OC(═O)Cc1cccc(OCn2nc(nc2c3ccc(nc3)c4ccccc4)c5ccc(nc5)N6CCOCC6)c1
OC(═O)Cc1ccc(OCc2ccc(c3ccc(cc3)c4ccccc4)c(n2)c5ccc(nc5)c6ccccc6)cc1
CSc1ccc(cc1)c2cncc(COc3ccc(CC(═O)O)cc3)c2c4ccc(OC(F)(F)F)cc4
CC(C)Oc1ccc(cc1)c2cc(cnc2COc3cccc(CC(═O)O)c3)c4ccccc4
OC(═O)Cc1ccc(OCc2nncc(c3ccc(cc3)N4CCOCC4)c2c5cccc(OC(F)(F)F)c5)cc1
CC(C)Oc1ccc(cc1)c2cnc(nc2COc3ccc(C)c(OCC(═O)O)c3)c4cncc(OC(F)(F)F)c4
COc1ncc(cc1F)c2cnc(COc3ccc(C)c(OCC(═O)O)c3)nc2c4ccc(SC)cc4
COc1ccncc1c2nc(COc3ccc(CC(═O)O)cc3)ncc2c4ccc(cc4)C(F)(F)F
COc1ccccc1c2nc(ncc2COc3cccc(CC(═O)O)c3)c4ccccc4
Cc1ccc(OCc2nnc(c3ccc(nc3)c4ccccc4)c(n2)c5cccnc5)cc1OCC(═O)O
OC(═O)Cc1ccc(OCc2nnc(c3ccc(OC(F)(F)F)cc3)c(n2)c4ccc(OC(F)(F)F)cc4)cc1
OC(═O)Cc1cccc(OCc2nc(nnc2c3ccc(cc3)c4ccccc4)c5ccc(cc5)c6ccccc6)c1
Cc1ccc(OCc2ccc(c3ccc(cc3)c4ccccc4)c(c2)c5ccc(cc5)c6ccccc6)cc1OCC(═O)O
COc1cncc(c1)c2c(COc3ccc(C)c(OCC(═O)O)c3)cccc2c4ccc(cc4)C(F)(F)F
CSc1ccc(cc1)c2cc(COc3ccc(CC(═O)O)cc3)cc(c2)c4ccc(cc4)c5ccccc5
OC(═O)Cc1ccc(OCc2ccc(cc2c3ccc(cc3)N4CCOCC4)c5ccc(cc5)C(F)(F)F)cc1
COc1ncc(cc1F)c2cc(ccc2COc3ccc(CC(═O)O)cc3)c4cccc(OC(F)(F)F)c4
COc1cncc(c1)c2ccc(COc3ccc(CC(═O)O)cc3)cc2c4ccc(SC)nc4
COc1ccc(cc1F)c2cccc(COc3cccc(CC(═O)O)c3)c2c4cccnc4
COc1ccc(cc1F)n2c(COc3ccc(C)c(OCC(═O)O)c3)nnc2c4ccc(OC(F)(F)F)nc4
CSc1ccc(cc1)n2c(COc3ccc(CC(═O)O)cc3)nnc2c4ccc(OC(C)C)cc4
COc1ccccc1c2nnc(c3ccc(cc3)C(F)(F)F)n2COc4cccc(CC(═O)O)c4
COc1cccc(c1)c2snc(c3cccc(CC(═O)O)c3)c2C(═O)NCCOc4ccc(C1)cc4C1
COc1ccc(cc1)c2snc(c3cccc(CC(═O)O)c3)c2C(═O)NCCOc4ccc(C1)cc4C1
OC(═O)Cc1cccc(c1)c2nsc(c3cccc(c3)C(F)(F)F)c2C(═O)NCCOc4ccc(C1)cc4C1
OC(═O)Cc1cccc(c1)c2nsc(c3ccc(F)cc3)c2C(═O)NCCOc4ccc(C1)cc4C1
OC(═O)Cc1cccc(c1)c2nsc(c3ccc(C1)cc3)c2C(═O)NCCOc4ccc(C1)cc4C1
OC(═O)Cc1cccc(c1)c2nsc(c3ccc(cc3)C(F)(F)F)c2C(═O)NCCOc4ccc(C1)cc4C1
Cc1ccc(cc1)c2snc(c3cccc(CC(═O)O)c3)c2C(═O)NCCOc4ccc(C1)cc4C1
COc1cccc(c1)c2nsc(c3cccc(CC(═O)O)c3)c2C(═O)NCCOc4ccc(C1)cc4C1
COc1ccc(cc1)c2nsc(c3cccc(CC(═O)O)c3)c2C(═O)NCCOc4ccc(C1)cc4C1
OC(═O)Cc1cccc(c1)c2snc(c3cccc(c3)C(F)(F)F)c2C(═O)NCCOc4ccc(C1)cc4C1
OC(═O)Cc1cccc(c1)c2snc(c3ccc(F)cc3)c2C(═O)NCCOc4ccc(C1)cc4C1
OC(═O)Cc1cccc(c1)c2snc(c3ccc(C1)cc3)c2C(═O)NCCOc4ccc(C1)cc4C1
OC(═O)Cc1cccc(c1)c2snc(c3ccc(cc3)C(F)(F)F)c2C(═O)NCCOc4ccc(C1)cc4C1
Cc1ccc(cc1)c2nsc(c3cccc(CC(═O)O)c3)c2C(═O)NCCOc4ccc(C1)cc4C1
COc1cccc(c1)c2snc(c3ccc(CC(═O)O)cc3C1)c2C(═O)NCCOc4ccc(C1)cc4C1
COc1ccc(cc1)c2snc(c3ccc(CC(═O)O)cc3C1)c2C(═O)NCCOc4ccc(C1)cc4C1
OC(═O)Cc1ccc(c(C1)c1)c2nsc(c3cccc(c3)C(F)(F)F)c2C(═O)NCCOc4ccc(C1)cc4C1
OC(═O)Cc1ccc(c(C1)c1)c2nsc(c3ccc(F)cc3)c2C(═O)NCCOc4ccc(C1)cc4C1
OC(═O)Cc1ccc(c(C1)c1)c2nsc(c3ccc(C1)cc3)c2C(═O)NCCOc4ccc(C1)cc4C1
OC(═O)Cc1ccc(c(C1)c1)c2nsc(c3ccc(cc3)C(F)(F)F)c2C(═O)NCCOc4ccc(C1)cc4C1
Cc1ccc(cc1)c2snc(c3ccc(CC(═O)O)cc3C1)c2C(═O)NCCOc4ccc(C1)cc4C1
COc1cccc(c1)c2nsc(c3ccc(CC(═O)O)cc3C1)c2C(═O)NCCOc4ccc(C1)cc4C1
COc1ccc(cc1)c2nsc(c3ccc(CC(═O)O)cc3C1)c2C(═O)NCCOc4ccc(C1)cc4C1
OC(═O)Cc1ccc(c(C1)c1)c2snc(c3cccc(c3)C(F)(F)F)c2C(═O)NCCOc4ccc(C1)cc4C1
OC(═O)Cc1ccc(c(C1)c1)c2snc(c3ccc(F)cc3)c2C(═O)NCCOc4ccc(C1)cc4C1
OC(═O)Cc1ccc(c(C1)c1)c2snc(c3ccc(C1)cc3)c2C(═O)NCCOc4ccc(C1)cc4C1
OC(═O)Cc1ccc(c(C1)c1)c2snc(c3ccc(cc3)C(F)(F)F)c2C(═O)NCCOc4ccc(C1)cc4C1
Cc1ccc(cc1)c2nsc(c3ccc(CC(═O)O)cc3C1)c2C(═O)NCCOc4ccc(C1)cc4C1
COc1cccc(c1)c2csc(c3cccc(CC(═O)O)c3)c2C(═O)NCCOc4ccc(C1)cc4C1
COc1ccc(cc1)c2csc(c3cccc(CC(═O)O)c3)c2C(═O)NCCOc4ccc(C1)cc4C1
OC(═O)Cc1cccc(c1)c2scc(c3cccc(c3)C(F)(F)F)c2C(═O)NCCOc4ccc(C1)cc4C1
OC(═O)Cc1cccc(c1)c2scc(c3ccc(F)cc3)c2C(═O)NCCOc4ccc(C1)cc4C1
OC(═O)Cc1cccc(c1)c2scc(c3ccc(C1)cc3)c2C(═O)NCCOc4ccc(C1)cc4C1
OC(═O)Cc1cccc(c1)c2scc(c3ccc(cc3)C(F)(F)F)c2C(═O)NCCOc4ccc(C1)cc4C1
Cc1ccc(cc1)c2csc(c3cccc(CC(═O)O)c3)c2C(═O)NCCOc4ccc(C1)cc4C1
COc1cccc(c1)c2scc(c3cccc(CC(═O)O)c3)c2C(═O)NCCOc4ccc(C1)cc4C1
COc1ccc(cc1)c2scc(c3cccc(CC(═O)O)c3)c2C(═O)NCCOc4ccc(C1)cc4C1
OC(═O)Cc1cccc(c1)c2csc(c3cccc(c3)C(F)(F)F)c2C(═O)NCCOc4ccc(C1)cc4C1
OC(═O)Cc1cccc(c1)c2csc(c3ccc(F)cc3)c2C(═O)NCCOc4ccc(C1)cc4C1
OC(═O)Cc1cccc(c1)c2csc(c3ccc(C1)cc3)c2C(═O)NCCOc4ccc(C1)cc4C1
OC(═O)Cc1cccc(c1)c2csc(c3ccc(cc3)C(F)(F)F)c2C(═O)NCCOc4ccc(C1)cc4C1
Cc1ccc(cc1)c2scc(c3cccc(CC(═O)O)c3)c2C(═O)NCCOc4ccc(C1)cc4C1
COc1cccc(c1)c2csc(c3ccc(CC(═O)O)cc3C1)c2C(═O)NCCOc4ccc(C1)cc4C1
COc1ccc(cc1)c2csc(c3ccc(CC(═O)O)cc3C1)c2C(═O)NCCOc4ccc(C1)cc4C1
OC(═O)Cc1ccc(c(C1)c1)c2scc(c3cccc(c3)C(F)(F)F)c2C(═O)NCCOc4ccc(C1)cc4C1
OC(═O)Cc1ccc(c(C1)c1)c2scc(c3ccc(F)cc3)c2C(═O)NCCOc4ccc(C1)cc4C1
OC(═O)Cc1ccc(c(C1)c1)c2scc(c3ccc(C1)cc3)c2C(═O)NCCOc4ccc(C1)cc4C1
OC(═O)Cc1ccc(c(C1)c1)c2scc(c3ccc(cc3)C(F)(F)F)c2C(═O)NCCOc4ccc(C1)cc4C1
Cc1ccc(cc1)c2csc(c3ccc(CC(═O)O)cc3C1)c2C(═O)NCCOc4ccc(C1)cc4C1
COc1cccc(c1)c2scc(c3ccc(CC(═O)O)cc3C1)c2C(═O)NCCOc4ccc(C1)cc4C1
COc1ccc(cc1)c2scc(c3ccc(CC(═O)O)cc3C1)c2C(═O)NCCOc4ccc(C1)cc4C1
OC(═O)Cc1ccc(c(C1)c1)c2csc(c3cccc(c3)C(F)(F)F)c2C(═O)NCCOc4ccc(C1)cc4C1
OC(═O)Cc1ccc(c(C1)c1)c2csc(c3ccc(F)cc3)c2C(═O)NCCOc4ccc(C1)cc4C1
OC(═O)Cc1ccc(c(C1)c1)c2csc(c3ccc(C1)cc3)c2C(═O)NCCOc4ccc(C1)cc4C1
OC(═O)Cc1ccc(c(C1)c1)c2csc(c3ccc(cc3)C(F)(F)F)c2C(═O)NCCOc4ccc(C1)cc4C1
Cc1ccc(cc1)c2scc(c3ccc(CC(═O)O)cc3C1)c2C(═O)NCCOc4ccc(C1)cc4C1
COc1cccc(c1)c2coc(c3cccc(CC(═O)O)c3)c2C(═O)NCCOc4ccc(C1)cc4C1
COc1ccc(cc1)c2coc(c3cccc(CC(═O)O)c3)c2C(═O)NCCOc4ccc(C1)cc4C1
OC(═O)Cc1cccc(c1)c2occ(c3cccc(c3)C(F)(F)F)c2C(═O)NCCOc4ccc(C1)cc4C1
OC(═O)Cc1cccc(c1)c2occ(c3ccc(F)cc3)c2C(═O)NCCOc4ccc(C1)cc4C1
OC(═O)Cc1cccc(c1)c2occ(c3ccc(C1)cc3)c2C(═O)NCCOc4ccc(C1)cc4C1
OC(═O)Cc1cccc(c1)c2occ(c3ccc(cc3)C(F)(F)F)c2C(═O)NCCOc4ccc(C1)cc4C1
Cc1ccc(cc1)c2coc(c3cccc(CC(═O)O)c3)c2C(═O)NCCOc4ccc(C1)cc4C1
COc1cccc(c1)c2occ(c3cccc(CC(═O)O)c3)c2C(═O)NCCOc4ccc(C1)cc4C1
COc1ccc(cc1)c2occ(c3cccc(CC(═O)O)c3)c2C(═O)NCCOc4ccc(C1)cc4C1
OC(═O)Cc1cccc(c1)c2coc(c3cccc(c3)C(F)(F)F)c2C(═O)NCCOc4ccc(C1)cc4C1
OC(═O)Cc1cccc(c1)c2coc(c3ccc(F)cc3)c2C(═O)NCCOc4ccc(C1)cc4C1
OC(═O)Cc1cccc(c1)c2coc(c3ccc(C1)cc3)c2C(═O)NCCOc4ccc(C1)cc4C1
OC(═O)Cc1cccc(c1)c2coc(c3ccc(cc3)C(F)(F)F)c2C(═O)NCCOc4ccc(C1)cc4C1
Cc1ccc(cc1)c2occ(c3cccc(CC(═O)O)c3)c2C(═O)NCCOc4ccc(C1)cc4C1
COc1cccc(c1)c2coc(c3ccc(CC(═O)O)cc3C1)c2C(═O)NCCOc4ccc(C1)cc4C1
COc1ccc(cc1)c2coc(c3ccc(CC(═O)O)cc3C1)c2C(═O)NCCOc4ccc(C1)cc4C1
OC(═O)Cc1ccc(c(C1)c1)c2occ(c3cccc(c3)C(F)(F)F)c2C(═O)NCCOc4ccc(C1)cc4C1
OC(═O)Cc1ccc(c(C1)c1)c2occ(c3ccc(F)cc3)c2C(═O)NCCOc4ccc(C1)cc4C1
OC(═O)Cc1ccc(c(C1)c1)c2occ(c3ccc(C1)cc3)c2C(═O)NCCOc4ccc(C1)cc4C1
OC(═O)Cc1ccc(c(C1)c1)c2occ(c3ccc(cc3)C(F)(F)F)c2C(═O)NCCOc4ccc(C1)cc4C1
Cc1ccc(cc1)c2coc(c3ccc(CC(═O)O)cc3C1)c2C(═O)NCCOc4ccc(C1)cc4C1
COc1cccc(c1)c2occ(c3ccc(CC(═O)O)cc3C1)c2C(═O)NCCOc4ccc(C1)cc4C1
COc1ccc(cc1)c2occ(c3ccc(CC(═O)O)cc3C1)c2C(═O)NCCOc4ccc(C1)cc4C1
OC(═O)Cc1ccc(c(C1)c1)c2coc(c3cccc(c3)C(F)(F)F)c2C(═O)NCCOc4ccc(C1)cc4C1
OC(═O)Cc1ccc(c(C1)c1)c2coc(c3ccc(F)cc3)c2C(═O)NCCOc4ccc(C1)cc4C1
OC(═O)Cc1ccc(c(C1)c1)c2coc(c3ccc(C1)cc3)c2C(═O)NCCOc4ccc(C1)cc4C1
OC(═O)Cc1ccc(c(C1)c1)c2coc(c3ccc(cc3)C(F)(F)F)c2C(═O)NCCOc4ccc(C1)cc4C1
Cc1ccc(cc1)c2occ(c3ccc(CC(═O)O)cc3C1)c2C(═O)NCCOc4ccc(C1)cc4C1
COc1cccc(c1)c2ccc(c3cccc(CC(═O)O)c3)n2C(═O)NCCOc4ccc(C1)cc4C1
COc1cccc(c1)c2c[nH]c(c3cccc(CC(═O)O)c3)c2C(═O)NCCOc4ccc(C1)cc4C1
COc1ccc(cc1)c2ccc(c3cccc(CC(═O)O)c3)n2C(═O)NCCOc4ccc(C1)cc4C1
COc1ccc(cc1)c2c[nH]c(c3cccc(CC(═O)O)c3)c2C(═O)NCCOc4ccc(C1)cc4C1
OC(═O)Cc1cccc(c1)c2ccc(c3cccc(c3)C(F)(F)F)n2C(═O)NCCOc4ccc(C1)cc4C1
OC(═O)Cc1cccc(c1)c2[nH]cc(c3cccc(c3)C(F)(F)F)c2C(═O)NCCOc4ccc(C1)cc4C1
OC(═O)Cc1cccc(c1)c2ccc(c3ccc(F)cc3)n2C(═O)NCCOc4ccc(C1)cc4C1
OC(═O)Cc1cccc(c1)c2[nH]cc(c3ccc(F)cc3)c2C(═O)NCCOc4ccc(C1)cc4C1
OC(═O)Cc1cccc(c1)c2ccc(c3ccc(C1)cc3)n2C(═O)NCCOc4ccc(C1)cc4C1
OC(═O)Cc1cccc(c1)c2[nH]cc(c3ccc(C1)cc3)c2C(═O)NCCOc4ccc(C1)cc4C1
OC(═O)Cc1cccc(c1)c2ccc(c3ccc(cc3)C(F)(F)F)n2C(═O)NCCOc4ccc(C1)cc4C1
OC(═O)Cc1cccc(c1)c2[nH]cc(c3ccc(cc3)C(F)(F)F)c2C(═O)NCCOc4ccc(C1)cc4C1
Cc1ccc(cc1)c2ccc(c3cccc(CC(═O)O)c3)n2C(═O)NCCOc4ccc(C1)cc4C1
Cc1ccc(cc1)c2c[nH]c(c3cccc(CC(═O)O)c3)c2C(═O)NCCOc4ccc(C1)cc4C1
COc1cccc(c1)c2ccn(c3cccc(CC(═O)O)c3)c2C(═O)NCCOc4ccc(C1)cc4C1
COc1ccc(cc1)c2ccn(c3cccc(CC(═O)O)c3)c2C(═O)NCCOc4ccc(C1)cc4C1
OC(═O)Cc1cccc(c1)n2ccc(c3cccc(c3)C(F)(F)F)c2C(═O)NCCOc4ccc(C1)cc4C1
OC(═O)Cc1cccc(c1)n2ccc(c3ccc(F)cc3)c2C(═O)NCCOc4ccc(C1)cc4C1
OC(═O)Cc1cccc(c1)n2ccc(c3ccc(C1)cc3)c2C(═O)NCCOc4ccc(C1)cc4C1
OC(═O)Cc1cccc(c1)n2ccc(c3ccc(cc3)C(F)(F)F)c2C(═O)NCCOc4ccc(C1)cc4C1
Cc1ccc(cc1)c2ccn(c3cccc(CC(═O)O)c3)c2C(═O)NCCOc4ccc(C1)cc4C1
COc1cccc(c1)c2[nH]cc(c3cccc(CC(═O)O)c3)c2C(═O)NCCOc4ccc(C1)cc4C1
COc1cccc(c1)n2ccc(c3cccc(CC(═O)O)c3)c2C(═O)NCCOc4ccc(C1)cc4C1
COc1ccc(cc1)c2[nH]cc(c3cccc(CC(═O)O)c3)c2C(═O)NCCOc4ccc(C1)cc4C1
COc1ccc(cc1)n2ccc(c3cccc(CC(═O)O)c3)c2C(═O)NCCOc4ccc(C1)cc4C1
OC(═O)Cc1cccc(c1)c2c[nH]c(c3cccc(c3)C(F)(F)F)c2C(═O)NCCOc4ccc(C1)cc4C1
OC(═O)Cc1cccc(c1)c2ccn(c3cccc(c3)C(F)(F)F)c2C(═O)NCCOc4ccc(C1)cc4C1
OC(═O)Cc1cccc(c1)c2c[nH]c(c3ccc(F)cc3)c2C(═O)NCCOc4ccc(C1)cc4C1
OC(═O)Cc1cccc(c1)c2ccn(c3ccc(F)cc3)c2C(═O)NCCOc4ccc(C1)cc4C1
OC(═O)Cc1cccc(c1)c2c[nH]c(c3ccc(C1)cc3)c2C(═O)NCCOc4ccc(C1)cc4C1
OC(═O)Cc1cccc(c1)c2ccn(c3ccc(C1)cc3)c2C(═O)NCCOc4ccc(C1)cc4C1
OC(═O)Cc1cccc(c1)c2c[nH]c(c3ccc(cc3)C(F)(F)F)c2C(═O)NCCOc4ccc(C1)cc4C1
OC(═O)Cc1cccc(c1)c2ccn(c3ccc(cc3)C(F)(F)F)c2C(═O)NCCOc4ccc(C1)cc4C1
Cc1ccc(cc1)c2[nH]cc(c3cccc(CC(═O)O)c3)c2C(═O)NCCOc4ccc(C1)cc4C1
Cc1ccc(cc1)n2ccc(c3cccc(CC(═O)O)c3)c2C(═O)NCCOc4ccc(C1)cc4C1
COc1cccc(c1)c2ccc(c3ccc(CC(═O)O)cc3C1)n2C(═O)NCCOc4ccc(C1)cc4C1
COc1cccc(c1)c2c[nH]c(c3ccc(CC(═O)O)cc3C1)c2C(═O)NCCOc4ccc(C1)cc4C1
COc1ccc(cc1)c2ccc(c3ccc(CC(═O)O)cc3C1)n2C(═O)NCCOc4ccc(C1)cc4C1
COc1ccc(cc1)c2c[nH]c(c3ccc(CC(═O)O)cc3C1)c2C(═O)NCCOc4ccc(C1)cc4C1
OC(═O)Cc1ccc(c(C1)c1)c2ccc(c3cccc(c3)C(F)(F)F)n2C(═O)NCCOc4ccc(C1)cc4C1
OC(═O)Cc1ccc(c(C1)c1)c2[nH]cc(c3cccc(c3)C(F)(F)F)c2C(═O)NCCOc4ccc(C1)cc4C1
OC(═O)Cc1ccc(c(C1)c1)c2ccc(c3ccc(F)cc3)n2C(═O)NCCOc4ccc(C1)cc4C1
OC(═O)Cc1ccc(c(C1)c1)c2[nH]cc(c3ccc(F)cc3)c2C(═O)NCCOc4ccc(C1)cc4C1
OC(═O)Cc1ccc(c(C1)c1)c2ccc(c3ccc(C1)cc3)n2C(═O)NCCOc4ccc(C1)cc4C1
OC(═O)Cc1ccc(c(C1)c1)c2[nH]cc(c3ccc(C1)cc3)c2C(═O)NCCOc4ccc(C1)cc4C1
OC(═O)Cc1ccc(c(C1)c1)c2ccc(c3ccc(cc3)C(F)(F)F)n2C(═O)NCCOc4ccc(C1)cc4C1
OC(═O)Cc1ccc(c(C1)c1)c2[nH]cc(c3ccc(cc3)C(F)(F)F)c2C(═O)NCCOc4ccc(C1)cc4C1
Cc1ccc(cc1)c2ccc(c3ccc(CC(═O)O)cc3C1)n2C(═O)NCCOc4ccc(C1)cc4C1
Cc1ccc(cc1)c2c[nH]c(c3ccc(CC(═O)O)cc3C1)c2C(═O)NCCOc4ccc(C1)cc4C1
COc1cccc(c1)c2ccn(c3ccc(CC(═O)O)cc3C1)c2C(═O)NCCOc4ccc(C1)cc4C1
COc1ccc(cc1)c2ccn(c3ccc(CC(═O)O)cc3C1)c2C(═O)NCCOc4ccc(C1)cc4C1
OC(═O)Cc1ccc(c(C1)c1)n2ccc(c3cccc(c3)C(F)(F)F)c2C(═O)NCCOc4ccc(C1)cc4C1
OC(═O)Cc1ccc(c(C1)c1)n2ccc(c3ccc(F)cc3)c2C(═O)NCCOc4ccc(C1)cc4C1
OC(═O)Cc1ccc(c(C1)c1)n2ccc(c3ccc(C1)cc3)c2C(═O)NCCOc4ccc(C1)cc4C1
OC(═O)Cc1ccc(c(C1)c1)n2ccc(c3ccc(cc3)C(F)(F)F)c2C(═O)NCCOc4ccc(C1)cc4C1
Cc1ccc(cc1)c2ccn(c3ccc(CC(═O)O)cc3C1)c2C(═O)NCCOc4ccc(C1)cc4C1
COc1cccc(c1)c2[nH]cc(c3ccc(CC(═O)O)cc3C1)c2C(═O)NCCOc4ccc(C1)cc4C1
COc1cccc(c1)n2ccc(c3ccc(CC(═O)O)cc3C1)c2C(═O)NCCOc4ccc(C1)cc4C1
COc1ccc(cc1)c2[nH]cc(c3ccc(CC(═O)O)cc3C1)c2C(═O)NCCOc4ccc(C1)cc4C1
COc1ccc(cc1)n2ccc(c3ccc(CC(═O)O)cc3C1)c2C(═O)NCCOc4ccc(C1)cc4C1
OC(═O)Cc1ccc(c(C1)c1)c2c[nH]c(c3cccc(c3)C(F)(F)F)c2C(═O)NCCOc4ccc(C1)cc4C1
OC(═O)Cc1ccc(c(C1)c1)c2ccn(c3cccc(c3)C(F)(F)F)c2C(═O)NCCOc4ccc(C1)cc4C1
OC(═O)Cc1ccc(c(C1)c1)c2c[nH]c(c3ccc(F)cc3)c2C(═O)NCCOc4ccc(C1)cc4C1
OC(═O)Cc1ccc(c(C1)c1)c2ccn(c3ccc(F)cc3)c2C(═O)NCCOc4ccc(C1)cc4C1
OC(═O)Cc1ccc(c(C1)c1)c2c[nH]c(c3ccc(C1)cc3)c2C(═O)NCCOc4ccc(C1)cc4C1
OC(═O)Cc1ccc(c(C1)c1)c2ccn(c3ccc(C1)cc3)c2C(═O)NCCOc4ccc(C1)cc4C1
OC(═O)Cc1ccc(c(C1)c1)c2c[nH]c(c3ccc(cc3)C(F)(F)F)c2C(═O)NCCOc4ccc(C1)cc4C1
OC(═O)Cc1ccc(c(C1)c1)c2ccn(c3ccc(cc3)C(F)(F)F)c2C(═O)NCCOc4ccc(C1)cc4C1
Cc1ccc(cc1)c2[nH]cc(c3ccc(CC(═O)O)cc3C1)c2C(═O)NCCOc4ccc(C1)cc4C1
Cc1ccc(cc1)n2ccc(c3ccc(CC(═O)O)cc3C1)c2C(═O)NCCOc4ccc(C1)cc4C1
COc1cccc(c1)c2n[nH]c(c3cccc(CC(═O)O)c3)c2C(═O)NCCOc4ccc(C1)cc4C1
COc1ccc(cc1)c2n[nH]c(c3cccc(CC(═O)O)c3)c2C(═O)NCCOc4ccc(C1)cc4C1
OC(═O)Cc1cccc(c1)c2[nH]nc(c3cccc(c3)C(F)(F)F)c2C(═O)NCCOc4ccc(C1)cc4C1
OC(═O)Cc1cccc(c1)c2[nH]nc(c3ccc(F)cc3)c2C(═O)NCCOc4ccc(C1)cc4C1
OC(═O)Cc1cccc(c1)c2[nH]nc(c3ccc(C1)cc3)c2C(═O)NCCOc4ccc(C1)cc4C1
OC(═O)Cc1cccc(c1)c2[nH]nc(c3ccc(cc3)C(F)(F)F)c2C(═O)NCCOc4ccc(C1)cc4C1
Cc1ccc(cc1)c2n[nH]c(c3cccc(CC(═O)O)c3)c2C(═O)NCCOc4ccc(C1)cc4C1
COc1cccc(c1)c2cnn(c3cccc(CC(═O)O)c3)c2C(═O)NCCOc4ccc(C1)cc4C1
COc1ccc(cc1)c2cnn(c3cccc(CC(═O)O)c3)c2C(═O)NCCOc4ccc(C1)cc4C1
OC(═O)Cc1cccc(c1)n2ncc(c3cccc(c3)C(F)(F)F)c2C(═O)NCCOc4ccc(C1)cc4C1
OC(═O)Cc1cccc(c1)n2ncc(c3ccc(F)cc3)c2C(═O)NCCOc4ccc(C1)cc4C1
OC(═O)Cc1cccc(c1)n2ncc(c3ccc(C1)cc3)c2C(═O)NCCOc4ccc(C1)cc4C1
OC(═O)Cc1cccc(c1)n2ncc(c3ccc(cc3)C(F)(F)F)c2C(═O)NCCOc4ccc(C1)cc4C1
Cc1ccc(cc1)c2cnn(c3cccc(CC(═O)O)c3)c2C(═O)NCCOc4ccc(C1)cc4C1
COc1cccc(c1)c2[nH]nc(c3cccc(CC(═O)O)c3)c2C(═O)NCCOc4ccc(C1)cc4C1
COc1ccc(cc1)c2[nH]nc(c3cccc(CC(═O)O)c3)c2C(═O)NCCOc4ccc(C1)cc4C1
OC(═O)Cc1cccc(c1)c2n[nH]c(c3cccc(c3)C(F)(F)F)c2C(═O)NCCOc4ccc(C1)cc4C1
OC(═O)Cc1cccc(c1)c2n[nH]c(c3ccc(F)cc3)c2C(═O)NCCOc4ccc(C1)cc4C1
OC(═O)Cc1cccc(c1)c2n[nH]c(c3ccc(C1)cc3)c2C(═O)NCCOc4ccc(C1)cc4C1
OC(═O)Cc1cccc(c1)c2n[nH]c(c3ccc(cc3)C(F)(F)F)c2C(═O)NCCOc4ccc(C1)cc4C1
Cc1ccc(cc1)c2[nH]nc(c3cccc(CC(═O)O)c3)c2C(═O)NCCOc4ccc(C1)cc4C1
COc1cccc(c1)n2ncc(c3cccc(CC(═O)O)c3)c2C(═O)NCCOc4ccc(C1)cc4C1
COc1ccc(cc1)n2ncc(c3cccc(CC(═O)O)c3)c2C(═O)NCCOc4ccc(C1)cc4C1
OC(═O)Cc1cccc(c1)c2cnn(c3cccc(c3)C(F)(F)F)c2C(═O)NCCOc4ccc(C1)cc4C1
OC(═O)Cc1cccc(c1)c2cnn(c3ccc(F)cc3)c2C(═O)NCCOc4ccc(C1)cc4C1
OC(═O)Cc1cccc(c1)c2cnn(c3ccc(C1)cc3)c2C(═O)NCCOc4ccc(C1)cc4C1
OC(═O)Cc1cccc(c1)c2cnn(c3ccc(cc3)C(F)(F)F)c2C(═O)NCCOc4ccc(C1)cc4C1
Cc1ccc(cc1)n2ncc(c3cccc(CC(═O)O)c3)c2C(═O)NCCOc4ccc(C1)cc4C1
COc1cccc(c1)c2n[nH]c(c3ccc(CC(═O)O)cc3C1)c2C(═O)NCCOc4ccc(C1)cc4C1
COc1ccc(cc1)c2n[nH]c(c3ccc(CC(═O)O)cc3C1)c2C(═O)NCCOc4ccc(C1)cc4C1
OC(═O)Cc1ccc(c(C1)c1)c2[nH]nc(c3cccc(c3)C(F)(F)F)c2C(═O)NCCOc4ccc(C1)cc4C1
OC(═O)Cc1ccc(c(C1)c1)c2[nH]nc(c3ccc(F)cc3)c2C(═O)NCCOc4ccc(C1)cc4C1
OC(═O)Cc1ccc(c(C1)c1)c2[nH]nc(c3ccc(C1)cc3)c2C(═O)NCCOc4ccc(C1)cc4C1
OC(═O)Cc1ccc(c(C1)c1)c2[nH]nc(c3ccc(cc3)C(F)(F)F)c2C(═O)NCCOc4ccc(C1)cc4C1
Cc1ccc(cc1)c2n[nH]c(c3ccc(CC(═O)O)cc3C1)c2C(═O)NCCOc4ccc(C1)cc4C1
COc1cccc(c1)c2cnn(c3ccc(CC(═O)O)cc3C1)c2C(═O)NCCOc4ccc(C1)cc4C1
COc1ccc(cc1)c2cnn(c3ccc(CC(═O)O)cc3C1)c2C(═O)NCCOc4ccc(C1)cc4C1
OC(═O)Cc1ccc(c(C1)c1)n2ncc(c3cccc(c3)C(F)(F)F)c2C(═O)NCCOc4ccc(C1)cc4C1
OC(═O)Cc1ccc(c(C1)c1)n2ncc(c3ccc(F)cc3)c2C(═O)NCCOc4ccc(C1)cc4C1
OC(═O)Cc1ccc(c(C1)c1)n2ncc(c3ccc(C1)cc3)c2C(═O)NCCOc4ccc(C1)cc4C1
OC(═O)Cc1ccc(c(C1)c1)n2ncc(c3ccc(cc3)C(F)(F)F)c2C(═O)NCCOc4ccc(C1)cc4C1
Cc1ccc(cc1)c2cnn(c3ccc(CC(═O)O)cc3C1)c2C(═O)NCCOc4ccc(C1)cc4C1
COc1cccc(c1)c2[nH]nc(c3ccc(CC(═O)O)cc3C1)c2C(═O)NCCOc4ccc(C1)cc4C1
COc1ccc(cc1)c2[nH]nc(c3ccc(CC(═O)O)cc3C1)c2C(═O)NCCOc4ccc(C1)cc4C1
OC(═O)Cc1ccc(c(C1)c1)c2n[nH]c(c3cccc(c3)C(F)(F)F)c2C(═O)NCCOc4ccc(C1)cc4C1
OC(═O)Cc1ccc(c(C1)c1)c2n[nH]c(c3ccc(F)cc3)c2C(═O)NCCOc4ccc(C1)cc4C1
OC(═O)Cc1ccc(c(C1)c1)c2n[nH]c(c3ccc(C1)cc3)c2C(═O)NCCOc4ccc(C1)cc4C1
OC(═O)Cc1ccc(c(C1)c1)c2n[nH]c(c3ccc(cc3)C(F)(F)F)c2C(═O)NCCOc4ccc(C1)cc4C1
Cc1ccc(cc1)c2[nH]nc(c3ccc(CC(═O)O)cc3C1)c2C(═O)NCCOc4ccc(C1)cc4C1
COc1cccc(c1)n2ncc(c3ccc(CC(═O)O)cc3C1)c2C(═O)NCCOc4ccc(C1)cc4C1
COc1ccc(cc1)n2ncc(c3ccc(CC(═O)O)cc3C1)c2C(═O)NCCOc4ccc(C1)cc4C1
OC(═O)Cc1ccc(c(C1)c1)c2cnn(c3cccc(c3)C(F)(F)F)c2C(═O)NCCOc4ccc(C1)cc4C1
OC(═O)Cc1ccc(c(C1)c1)c2cnn(c3ccc(F)cc3)c2C(═O)NCCOc4ccc(C1)cc4C1
OC(═O)Cc1ccc(c(C1)c1)c2cnn(c3ccc(C1)cc3)c2C(═O)NCCOc4ccc(C1)cc4C1
OC(═O)Cc1ccc(c(C1)c1)c2cnn(c3ccc(cc3)C(F)(F)F)c2C(═O)NCCOc4ccc(C1)cc4C1
Cc1ccc(cc1)n2ncc(c3ccc(CC(═O)O)cc3C1)c2C(═O)NCCOc4ccc(C1)cc4C1
COc1cccc(c1)c2ncc(c3cccc(CC(═O)O)c3)n2C(═O)NCCOc4ccc(C1)cc4C1
COc1ccc(cc1)c2ncc(c3cccc(CC(═O)O)c3)n2C(═O)NCCOc4ccc(C1)cc4C1
OC(═O)Cc1cccc(c1)c2cnc(c3cccc(c3)C(F)(F)F)n2C(═O)NCCOc4ccc(C1)cc4C1
OC(═O)Cc1cccc(c1)c2cnc(c3ccc(F)cc3)n2C(═O)NCCOc4ccc(C1)cc4C1
OC(═O)Cc1cccc(c1)c2cnc(c3ccc(C1)cc3)n2C(═O)NCCOc4ccc(C1)cc4C1
OC(═O)Cc1cccc(c1)c2cnc(c3ccc(cc3)C(F)(F)F)n2C(═O)NCCOc4ccc(C1)cc4C1
Cc1ccc(cc1)c2ncc(c3cccc(CC(═O)O)c3)n2C(═O)NCCOc4ccc(C1)cc4C1
COc1cccc(c1)c2ncn(c3cccc(CC(═O)O)c3)c2C(═O)NCCOc4ccc(C1)cc4C1
COc1ccc(cc1)c2ncn(c3cccc(CC(═O)O)c3)c2C(═O)NCCOc4ccc(C1)cc4C1
OC(═O)Cc1cccc(c1)n2cnc(c3cccc(c3)C(F)(F)F)c2C(═O)NCCOc4ccc(C1)cc4C1
OC(═O)Cc1cccc(c1)n2cnc(c3ccc(F)cc3)c2C(═O)NCCOc4ccc(C1)cc4C1
OC(═O)Cc1cccc(c1)n2cnc(c3ccc(C1)cc3)c2C(═O)NCCOc4ccc(C1)cc4C1
OC(═O)Cc1cccc(c1)n2cnc(c3ccc(cc3)C(F)(F)F)c2C(═O)NCCOc4ccc(C1)cc4C1
Cc1ccc(cc1)c2ncn(c3cccc(CC(═O)O)c3)c2C(═O)NCCOc4ccc(C1)cc4C1
COc1cccc(c1)c2cnc(c3cccc(CC(═O)O)c3)n2C(═O)NCCOc4ccc(C1)cc4C1
COc1ccc(cc1)c2cnc(c3cccc(CC(═O)O)c3)n2C(═O)NCCOc4ccc(C1)cc4C1
OC(═O)Cc1cccc(c1)c2ncc(c3cccc(c3)C(F)(F)F)n2C(═O)NCCOc4ccc(C1)cc4C1
OC(═O)Cc1cccc(c1)c2ncc(c3ccc(F)cc3)n2C(═O)NCCOc4ccc(C1)cc4C1
OC(═O)Cc1cccc(c1)c2ncc(c3ccc(C1)cc3)n2C(═O)NCCOc4ccc(C1)cc4C1
OC(═O)Cc1cccc(c1)c2ncc(c3ccc(cc3)C(F)(F)F)n2C(═O)NCCOc4ccc(C1)cc4C1
Cc1ccc(cc1)c2cnc(c3cccc(CC(═O)O)c3)n2C(═O)NCCOc4ccc(C1)cc4C1
COc1cccc(c1)n2cnc(c3cccc(CC(═O)O)c3)c2C(═O)NCCOc4ccc(C1)cc4C1
COc1ccc(cc1)n2cnc(c3cccc(CC(═O)O)c3)c2C(═O)NCCOc4ccc(C1)cc4C1
OC(═O)Cc1cccc(c1)c2ncn(c3cccc(c3)C(F)(F)F)c2C(═O)NCCOc4ccc(C1)cc4C1
OC(═O)Cc1cccc(c1)c2ncn(c3ccc(F)cc3)c2C(═O)NCCOc4ccc(C1)cc4C1
OC(═O)Cc1cccc(c1)c2ncn(c3ccc(C1)cc3)c2C(═O)NCCOc4ccc(C1)cc4C1
OC(═O)Cc1cccc(c1)c2ncn(c3ccc(cc3)C(F)(F)F)c2C(═O)NCCOc4ccc(C1)cc4C1
Cc1ccc(cc1)n2cnc(c3cccc(CC(═O)O)c3)c2C(═O)NCCOc4ccc(C1)cc4C1
COc1cccc(c1)c2ncc(c3ccc(CC(═O)O)cc3C1)n2C(═O)NCCOc4ccc(C1)cc4C1
COc1ccc(cc1)c2ncc(c3ccc(CC(═O)O)cc3C1)n2C(═O)NCCOc4ccc(C1)cc4C1
OC(═O)Cc1ccc(c(C1)c1)c2cnc(c3cccc(c3)C(F)(F)F)n2C(═O)NCCOc4ccc(C1)cc4C1
OC(═O)Cc1ccc(c(C1)c1)c2cnc(c3ccc(F)cc3)n2C(═O)NCCOc4ccc(C1)cc4C1
OC(═O)Cc1ccc(c(C1)c1)c2cnc(c3ccc(C1)cc3)n2C(═O)NCCOc4ccc(C1)cc4C1
OC(═O)Cc1ccc(c(C1)c1)c2cnc(c3ccc(cc3)C(F)(F)F)n2C(═O)NCCOc4ccc(C1)cc4C1
Cc1ccc(cc1)c2ncc(c3ccc(CC(═O)O)cc3C1)n2C(═O)NCCOc4ccc(C1)cc4C1
COc1cccc(c1)c2ncn(c3ccc(CC(═O)O)cc3C1)c2C(═O)NCCOc4ccc(C1)cc4C1
COc1ccc(cc1)c2ncn(c3ccc(CC(═O)O)cc3C1)c2C(═O)NCCOc4ccc(C1)cc4C1
OC(═O)Cc1ccc(c(C1)c1)n2cnc(c3cccc(c3)C(F)(F)F)c2C(═O)NCCOc4ccc(C1)cc4C1
OC(═O)Cc1ccc(c(C1)c1)n2cnc(c3ccc(F)cc3)c2C(═O)NCCOc4ccc(C1)cc4C1
OC(═O)Cc1ccc(c(C1)c1)n2cnc(c3ccc(C1)cc3)c2C(═O)NCCOc4ccc(C1)cc4C1
OC(═O)Cc1ccc(c(C1)c1)n2cnc(c3ccc(cc3)C(F)(F)F)c2C(═O)NCCOc4ccc(C1)cc4C1
Cc1ccc(cc1)c2ncn(c3ccc(CC(═O)O)cc3C1)c2C(═O)NCCOc4ccc(C1)cc4C1
COc1cccc(c1)c2cnc(c3ccc(CC(═O)O)cc3C1)n2C(═O)NCCOc4ccc(C1)cc4C1
COc1ccc(cc1)c2cnc(c3ccc(CC(═O)O)cc3C1)n2C(═O)NCCOc4ccc(C1)cc4C1
OC(═O)Cc1ccc(c(C1)c1)c2ncc(c3cccc(c3)C(F)(F)F)n2C(═O)NCCOc4ccc(C1)cc4C1
OC(═O)Cc1ccc(c(C1)c1)c2ncc(c3ccc(F)cc3)n2C(═O)NCCOc4ccc(C1)cc4C1
OC(═O)Cc1ccc(c(C1)c1)c2ncc(c3ccc(C1)cc3)n2C(═O)NCCOc4ccc(C1)cc4C1
OC(═O)Cc1ccc(c(C1)c1)c2ncc(c3ccc(cc3)C(F)(F)F)n2C(═O)NCCOc4ccc(C1)cc4C1
Cc1ccc(cc1)c2cnc(c3ccc(CC(═O)O)cc3C1)n2C(═O)NCCOc4ccc(C1)cc4C1
COc1cccc(c1)n2cnc(c3ccc(CC(═O)O)cc3C1)c2C(═O)NCCOc4ccc(C1)cc4C1
COc1ccc(cc1)n2cnc(c3ccc(CC(═O)O)cc3C1)c2C(═O)NCCOc4ccc(C1)cc4C1
OC(═O)Cc1ccc(c(C1)c1)c2ncn(c3cccc(c3)C(F)(F)F)c2C(═O)NCCOc4ccc(C1)cc4C1
OC(═O)Cc1ccc(c(C1)c1)c2ncn(c3ccc(F)cc3)c2C(═O)NCCOc4ccc(C1)cc4C1
OC(═O)Cc1ccc(c(C1)c1)c2ncn(c3ccc(C1)cc3)c2C(═O)NCCOc4ccc(C1)cc4C1
OC(═O)Cc1ccc(c(C1)c1)c2ncn(c3ccc(cc3)C(F)(F)F)c2C(═O)NCCOc4ccc(C1)cc4C1
Cc1ccc(cc1)n2cnc(c3ccc(CC(═O)O)cc3C1)c2C(═O)NCCOc4ccc(C1)cc4C1
COc1cccc(c1)c2nnn(c3cccc(CC(═O)O)c3)c2C(═O)NCCOc4ccc(C1)cc4C1
COc1ccc(cc1)c2nnn(c3cccc(CC(═O)O)c3)c2C(═O)NCCOc4ccc(C1)cc4C1
OC(═O)Cc1cccc(c1)n2nnc(c3cccc(c3)C(F)(F)F)c2C(═O)NCCOc4ccc(C1)cc4C1
OC(═O)Cc1cccc(c1)n2nnc(c3ccc(F)cc3)c2C(═O)NCCOc4ccc(C1)cc4C1
OC(═O)Cc1cccc(c1)n2nnc(c3ccc(C1)cc3)c2C(═O)NCCOc4ccc(C1)cc4C1
OC(═O)Cc1cccc(c1)n2nnc(c3ccc(cc3)C(F)(F)F)c2C(═O)NCCOc4ccc(C1)cc4C1
Cc1ccc(cc1)c2nnn(c3cccc(CC(═O)O)c3)c2C(═O)NCCOc4ccc(C1)cc4C1
COc1cccc(c1)n2nnc(c3cccc(CC(═O)O)c3)c2C(═O)NCCOc4ccc(C1)cc4C1
COc1ccc(cc1)n2nnc(c3cccc(CC(═O)O)c3)c2C(═O)NCCOc4ccc(C1)cc4C1
OC(═O)Cc1cccc(c1)c2nnn(c3cccc(c3)C(F)(F)F)c2C(═O)NCCOc4ccc(C1)cc4C1
OC(═O)Cc1cccc(c1)c2nnn(c3ccc(F)cc3)c2C(═O)NCCOc4ccc(C1)cc4C1
OC(═O)Cc1cccc(c1)c2nnn(c3ccc(C1)cc3)c2C(═O)NCCOc4ccc(C1)cc4C1
OC(═O)Cc1cccc(c1)c2nnn(c3ccc(cc3)C(F)(F)F)c2C(═O)NCCOc4ccc(C1)cc4C1
Cc1ccc(cc1)n2nnc(c3cccc(CC(═O)O)c3)c2C(═O)NCCOc4ccc(C1)cc4C1
COc1cccc(c1)c2nnn(c3ccc(CC(═O)O)cc3C1)c2C(═O)NCCOc4ccc(C1)cc4C1
COc1ccc(cc1)c2nnn(c3ccc(CC(═O)O)cc3C1)c2C(═O)NCCOc4ccc(C1)cc4C1
OC(═O)Cc1ccc(c(C1)c1)n2nnc(c3cccc(c3)C(F)(F)F)c2C(═O)NCCOc4ccc(C1)cc4C1
OC(═O)Cc1ccc(c(C1)c1)n2nnc(c3ccc(F)cc3)c2C(═O)NCCOc4ccc(C1)cc4C1
OC(═O)Cc1ccc(c(C1)c1)n2nnc(c3ccc(C1)cc3)c2C(═O)NCCOc4ccc(C1)cc4C1
OC(═O)Cc1ccc(c(C1)c1)n2nnc(c3ccc(cc3)C(F)(F)F)c2C(═O)NCCOc4ccc(C1)cc4C1
Cc1ccc(cc1)c2nnn(c3ccc(CC(═O)O)cc3C1)c2C(═O)NCCOc4ccc(C1)cc4C1
COc1cccc(c1)n2nnc(c3ccc(CC(═O)O)cc3C1)c2C(═O)NCCOc4ccc(C1)cc4C1
COc1ccc(cc1)n2nnc(c3ccc(CC(═O)O)cc3C1)c2C(═O)NCCOc4ccc(C1)cc4C1
OC(═O)Cc1ccc(c(C1)c1)c2nnn(c3cccc(c3)C(F)(F)F)c2C(═O)NCCOc4ccc(C1)cc4C1
OC(═O)Cc1ccc(c(C1)c1)c2nnn(c3ccc(F)cc3)c2C(═O)NCCOc4ccc(C1)cc4C1
OC(═O)Cc1ccc(c(C1)c1)c2nnn(c3ccc(C1)cc3)c2C(═O)NCCOc4ccc(C1)cc4C1
OC(═O)Cc1ccc(c(C1)c1)c2nnn(c3ccc(cc3)C(F)(F)F)c2C(═O)NCCOc4ccc(C1)cc4C1
Cc1ccc(cc1)n2nnc(c3ccc(CC(═O)O)cc3C1)c2C(═O)NCCOc4ccc(C1)cc4C1
COc1cccc(c1)c2nnc(c3cccc(CC(═O)O)c3)n2C(═O)NCCOc4ccc(C1)cc4C1
COc1ccc(cc1)c2nnc(c3cccc(CC(═O)O)c3)n2C(═O)NCCOc4ccc(C1)cc4C1
OC(═O)Cc1cccc(c1)c2nnc(c3cccc(c3)C(F)(F)F)n2C(═O)NCCOc4ccc(C1)cc4C1
OC(═O)Cc1cccc(c1)c2nnc(c3ccc(F)cc3)n2C(═O)NCCOc4ccc(C1)cc4C1
OC(═O)Cc1cccc(c1)c2nnc(c3ccc(C1)cc3)n2C(═O)NCCOc4ccc(C1)cc4C1
OC(═O)Cc1cccc(c1)c2nnc(c3ccc(cc3)C(F)(F)F)n2C(═O)NCCOc4ccc(C1)cc4C1
Cc1ccc(cc1)c2nnc(c3cccc(CC(═O)O)c3)n2C(═O)NCCOc4ccc(C1)cc4C1
COc1cccc(c1)c2nnc(c3ccc(CC(═O)O)cc3C1)n2C(═O)NCCOc4ccc(C1)cc4C1
COc1ccc(cc1)c2nnc(c3ccc(CC(═O)O)cc3C1)n2C(═O)NCCOc4ccc(C1)cc4C1
OC(═O)Cc1ccc(c(C1)c1)c2nnc(c3cccc(c3)C(F)(F)F)n2C(═O)NCCOc4ccc(C1)cc4C1
OC(═O)Cc1ccc(c(C1)c1)c2nnc(c3ccc(F)cc3)n2C(═O)NCCOc4ccc(C1)cc4C1
OC(═O)Cc1ccc(c(C1)c1)c2nnc(c3ccc(C1)cc3)n2C(═O)NCCOc4ccc(C1)cc4C1
OC(═O)Cc1ccc(c(C1)c1)c2nnc(c3ccc(cc3)C(F)(F)F)n2C(═O)NCCOc4ccc(C1)cc4C1
Cc1ccc(cc1)c2nnc(c3ccc(CC(═O)O)cc3C1)n2C(═O)NCCOc4ccc(C1)cc4C1
Cc1ccc(cc1OCC(═O)O)c2nsc(c3cccc(c3)C(F)(F)F)c2C(═O)NCCOc4ccc(C1)cc4C1
COc1cccc(c1)c2snc(c3ccc4CC(Cc4c3)C(═O)O)c2C(═O)NCCOc5ccc(C1)cc5C1
OC(═O)C1CCc2ccc(cc12)c3nsc(c4ccc(F)cc4)c3C(═O)NCCOc5ccc(C1)cc5C1
Cc1ccc(cc1)c2snc(c3ccc4CCN(CC(═O)O)c4c3)c2C(═O)NCCOc5ccc(C1)cc5C1
COc1ccc(cc1)c2snc(c3cccc4c(CC(═O)O)csc34)c2C(═O)NCCOc5ccc(C1)cc5C1
OC(═O)C1Cc2cc(ccc2O1)c3nsc(c4ccc(F)cc4)c3C(═O)NCCOc5ccc(C1)cc5C1
OC(═O)C1CCc2cccc(c2C1)c3nsc(c4ccc(cc4)C(F)(F)F)c3C(═O)NCCOc5ccc(C1)cc5C1
COc1cccc(c1)c2snc(c3cccc4CCC(CC(═O)O)Oc34)c2C(═O)NCCOc5ccc(C1)cc5C1
Cc1ccc(cc1)c2csc(c3ccc(C)c(OCC(═O)O)c3)c2C(═O)NCCOc4ccc(C1)cc4C1
OC(═O)C1Cc2cccc(c2C1)c3scc(c4ccc(F)cc4)c3C(═O)NCCOc5ccc(C1)cc5C1
COc1ccc(cc1)c2csc(c3ccc4c(ccn4CC(═O)O)c3)c2C(═O)NCCOc5ccc(C1)cc5C1
OC(═O)CC1Cc2cccc(c2C1)c3csc(c4cccc(c4)C(F)(F)F)c3C(═O)NCCOc5ccc(C1)cc5C1
COc1cccc(c1)c2csc(c3ccc4C(CC(═O)O)CCc4c3)c2C(═O)NCCOc5ccc(C1)cc5C1
OC(═O)C1Cc2cccc(c2O1)c3scc(c4ccc(cc4)C(F)(F)F)c3C(═O)NCCOc5ccc(C1)cc5C1
COc1cccc(c1)c2scc(c3cccc4CCC(Oc34)C(═O)O)c2C(═O)NCCOc5ccc(C1)cc5C1
OC(═O)CC1CCc2cc(ccc2O1)c3scc(c4ccc(F)cc4)c3C(═O)NCCOc5ccc(C1)cc5C1
Cc1ccc(cc1)c2occ(c3cccc4c(CC(═O)O)csc34)c2C(═O)NCCOc5ccc(C1)cc5C1
Cc1ccc(cc1OCC(═O)O)c2ccc(c3ccc(cc3)C(F)(F)F)n2C(═O)NCCOc4ccc(C1)cc4C1
Cc1ccc(cc1OCC(═O)O)c2c[nH]c(c3ccc(cc3)C(F)(F)F)c2C(═O)NCCOc4ccc(C1)cc4C1
Cc1ccc(cc1OCC(═O)O)c2ccn(c3ccc(cc3)C(F)(F)F)c2C(═O)NCCOc4ccc(C1)cc4C1
COc1cccc(c1)c2ccc(c3cccc4CC(Cc34)C(═O)O)n2C(═O)NCCOc5ccc(C1)cc5C1
COc1cccc(c1)c2ccn(c3cccc4CC(Cc34)C(═O)O)c2C(═O)NCCOc5ccc(C1)cc5C1
COc1cccc(c1)c2[nH]cc(c3cccc4CC(Cc34)C(═O)O)c2C(═O)NCCOc5ccc(C1)cc5C1
OC(═O)C1CCc2ccc(cc12)c3[nH]cc(c4ccc(cc4)C(F)(F)F)c3C(═O)NCCOc5ccc(C1)cc5C1
OC(═O)C1CCc2ccc(cc12)n3ccc(c4ccc(F)cc4)c3C(═O)NCCOc5ccc(C1)cc5C1
OC(═O)Cn1ccc2cc(ccc12)c3[nH]cc(c4cccc(c4)C(F)(F)F)c3C(═O)NCCOc5ccc(C1)cc5C1
OC(═O)Cn1ccc2cc(ccc12)c3ccc(c4ccc(cc4)C(F)(F)F)n3C(═O)NCCOc5ccc(C1)cc5C1
OC(═O)Cn1ccc2cc(ccc12)n3ccc(c4cccc(c4)C(F)(F)F)c3C(═O)NCCOc5ccc(C1)cc5C1
Cc1ccc(cc1)c2c[nH]c(c3cccc4CC(CC(═O)O)Cc34)c2C(═O)NCCOc5ccc(C1)cc5C1
Cc1ccc(cc1)c2ccc(c3ccc4C(CC(═O)O)CCc4c3)n2C(═O)NCCOc5ccc(C1)cc5C1
OC(═O)CN1CCc2ccc(cc12)c3ccc(c4ccc(F)cc4)n3C(═O)NCCOc5ccc(C1)cc5C1
Cc1ccc(cc1)c2ccn(c3ccc4CCN(CC(═O)O)c4c3)c2C(═O)NCCOc5ccc(C1)cc5C1
OC(═O)CN1CCc2ccc(cc12)c3c[nH]c(c4ccc(C1)cc4)c3C(═O)NCCOc5ccc(C1)cc5C1
COc1cccc(c1)c2c[nH]c(c3cccc4c(CC(═O)O)csc34)c2C(═O)NCCOc5ccc(C1)cc5C1
Cc1ccc(cc1)c2ccn(c3cccc4c(CC(═O)O)csc34)c2C(═O)NCCOc5ccc(C1)cc5C1
OC(═O)CC1Cc2cccc(c2O1)c3ccc(c4cccc(c4)C(F)(F)F)n3C(═O)NCCOc5ccc(C1)cc5C1
OC(═O)CC1Cc2cccc(c2O1)n3ccc(c4cccc(c4)C(F)(F)F)c3C(═O)NCCOc5ccc(C1)cc5C1
COc1cccc(c1)n2ccc(c3cccc4CCC(Oc34)C(═O)O)c2C(═O)NCCOc5ccc(C1)cc5C1
OC(═O)Cc1csc2ccc(cc12)c3[nH]cc(c4cccc(c4)C(F)(F)F)c3C(═O)NCCOc5ccc(C1)cc5C1
Cc1ccc(cc1)c2ccc(c3ccc4scc(CC(═O)O)c4c3)n2C(═O)NCCOc5ccc(C1)cc5C1
COc1ccc(cc1)c2[nH]cc(c3ccc4OC(Cc4c3)C(═O)O)c2C(═O)NCCOc5ccc(C1)cc5C1
COc1ccc(cc1)c2ccc(c3ccc4OC(CCc4c3)C(═O)O)n2C(═O)NCCOc5ccc(C1)cc5C1
OC(═O)C1CCc2cccc(c2C1)c3ccn(c4cccc(c4)C(F)(F)F)c3C(═O)NCCOc5ccc(C1)cc5C1
OC(═O)CC1CCc2cccc(c2O1)c3c[nH]c(c4ccc(cc4)C(F)(F)F)c3C(═O)NCCOc5ccc(C1)cc5C1
OC(═O)CC1CCc2cc(ccc2O1)n3ccc(c4ccc(F)cc4)c3C(═O)NCCOc5ccc(C1)cc5C1
COc1ccc(cc1)c2n[nH]c(c3ccc(C)c(OCC(═O)O)c3)c2C(═O)NCCOc4ccc(C1)cc4C1
Cc1ccc(cc1OCC(═O)O)n2ncc(c3ccc(C1)cc3)c2C(═O)NCCOc4ccc(C1)cc4C1
Cc1ccc(cc1)c2n[nH]c(c3cccc4CC(Cc34)C(═O)O)c2C(═O)NCCOc5ccc(C1)cc5C1
Cc1ccc(cc1)c2[nH]nc(c3ccc4c(ccn4CC(═O)O)c3)c2C(═O)NCCOc5ccc(C1)cc5C1
COc1cccc(c1)n2ncc(c3ccc4c(ccn4CC(═O)O)c3)c2C(═O)NCCOc5ccc(C1)cc5C1
OC(═O)CC1Cc2cccc(c2C1)c3cnn(c4cccc(c4)C(F)(F)F)c3C(═O)NCCOc5ccc(C1)cc5C1
OC(═O)CC1CCc2cc(ccc12)c3[nH]nc(c4ccc(cc4)C(F)(F)F)c3C(═O)NCCOc5ccc(C1)cc5C1
COc1ccc(cc1)c2cnn(c3ccc4C(CC(═O)O)CCc4c3)c2C(═O)NCCOc5ccc(C1)cc5C1
OC(═O)CN1CCc2ccc(cc12)c3cnn(c4ccc(cc4)C(F)(F)F)c3C(═O)NCCOc5ccc(C1)cc5C1
COc1cccc(c1)n2ncc(c3cccc4c(CC(═O)O)csc34)c2C(═O)NCCOc5ccc(C1)cc5C1
COc1cccc(c1)c2cnn(c3cccc4CC(Oc34)C(═O)O)c2C(═O)NCCOc5ccc(C1)cc5C1
OC(═O)C1CCc2cccc(c2O1)c3[nH]nc(c4cccc(c4)C(F)(F)F)c3C(═O)NCCOc5ccc(C1)cc5C1
Cc1ccc(cc1)c2[nH]nc(c3ccc4scc(CC(═O)O)c4c3)c2C(═O)NCCOc5ccc(C1)cc5C1
OC(═O)Cc1csc2ccc(cc12)c3cnn(c4ccc(cc4)C(F)(F)F)c3C(═O)NCCOc5ccc(C1)cc5C1
COc1ccc(cc1)c2[nH]nc(c3ccc4OC(CC(═O)O)Cc4c3)c2C(═O)NCCOc5ccc(C1)cc5C1
OC(═O)C1CCc2cc(ccc2O1)n3ncc(c4cccc(c4)C(F)(F)F)c3C(═O)NCCOc5ccc(C1)cc5C1
OC(═O)C1CCc2cccc(c2C1)n3ncc(c4ccc(F)cc4)c3C(═O)NCCOc5ccc(C1)cc5C1
Cc1ccc(cc1)n2ncc(c3cccc4CCC(CC(═O)O)Oc34)c2C(═O)NCCOc5ccc(C1)cc5C1
COc1ccc(cc1)c2ncc(c3ccc(C)c(OCC(═O)O)c3)n2C(═O)NCCOc4ccc(C1)cc4C1
COc1cccc(c1)n2cnc(c3ccc(C)c(OCC(═O)O)c3)c2C(═O)NCCOc4ccc(C1)cc4C1
OC(═O)C1Cc2cccc(c2C1)c3cnc(c4cccc(c4)C(F)(F)F)n3C(═O)NCCOc5ccc(C1)cc5C1
OC(═O)C1Cc2cccc(c2C1)n3cnc(c4cccc(c4)C(F)(F)F)c3C(═O)NCCOc5ccc(C1)cc5C1
OC(═O)C1CCc2ccc(cc12)c3ncc(c4ccc(F)cc4)n3C(═O)NCCOc5ccc(C1)cc5C1
COc1cccc(c1)c2ncc(c3ccc4c(ccn4CC(═O)O)c3)n2C(═O)NCCOc5ccc(C1)cc5C1
OC(═O)Cn1ccc2cc(ccc12)n3cnc(c4ccc(C1)cc4)c3C(═O)NCCOc5ccc(C1)cc5C1
Cc1ccc(cc1)c2cnc(c3cccc4CC(CC(═O)O)Cc34)n2C(═O)NCCOc5ccc(C1)cc5C1
Cc1ccc(cc1)n2cnc(c3cccc4CC(CC(═O)O)Cc34)c2C(═O)NCCOc5ccc(C1)cc5C1
COc1ccc(cc1)c2ncn(c3ccc4C(CC(═O)O)CCc4c3)c2C(═O)NCCOc5ccc(C1)cc5C1
OC(═O)CN1CCc2ccc(cc12)n3cnc(c4ccc(F)cc4)c3C(═O)NCCOc5ccc(C1)cc5C1
OC(═O)CN1CCc2ccc(cc12)c3ncc(c4ccc(cc4)C(F)(F)F)n3C(═O)NCCOc5ccc(C1)cc5C1
OC(═O)Cc1csc2c(cccc12)c3cnc(c4ccc(F)cc4)n3C(═O)NCCOc5ccc(C1)cc5C1
OC(═O)Cc1csc2c(cccc12)c3ncn(c4cccc(c4)C(F)(F)F)c3C(═O)NCCOc5ccc(C1)cc5C1
Cc1ccc(cc1)c2ncc(c3cccc4CC(CC(═O)O)Oc34)n2C(═O)NCCOc5ccc(C1)cc5C1
OC(═O)CC1Cc2cccc(c2O1)c3ncn(c4cccc(c4)C(F)(F)F)c3C(═O)NCCOc5ccc(C1)cc5C1
OC(═O)C1CCc2cccc(c2O1)n3cnc(c4ccc(C1)cc4)c3C(═O)NCCOc5ccc(C1)cc5C1
OC(═O)C1CCc2cccc(c2O1)c3ncc(c4cccc(c4)C(F)(F)F)n3C(═O)NCCOc5ccc(C1)cc5C1
COc1ccc(cc1)c2ncn(c3ccc4OC(Cc4c3)C(═O)O)c2C(═O)NCCOc5ccc(C1)cc5C1
OC(═O)C1Cc2cc(ccc2O1)c3ncc(c4ccc(F)cc4)n3C(═O)NCCOc5ccc(C1)cc5C1
Cc1ccc(cc1OCC(═O)O)n2nnc(c3cccc(c3)C(F)(F)F)c2C(═O)NCCOc4ccc(C1)cc4C1
Cc1ccc(cc1)c2nnn(c3cccc4CC(Cc34)C(═O)O)c2C(═O)NCCOc5ccc(C1)cc5C1
OC(═O)C1CCc2ccc(cc12)n3nnc(c4ccc(cc4)C(F)(F)F)c3C(═O)NCCOc5ccc(C1)cc5C1
OC(═O)Cn1ccc2cc(ccc12)c3nnn(c4ccc(cc4)C(F)(F)F)c3C(═O)NCCOc5ccc(C1)cc5C1
COc1ccc(cc1)n2nnc(c3cccc4CC(CC(═O)O)Cc34)c2C(═O)NCCOc5ccc(C1)cc5C1
COc1ccc(cc1)c2nnn(c3ccc4CCN(CC(═O)O)c4c3)c2C(═O)NCCOc5ccc(C1)cc5C1
OC(═O)Cc1csc2c(cccc12)n3nnc(c4ccc(C1)cc4)c3C(═O)NCCOc5ccc(C1)cc5C1
OC(═O)CC1Cc2cccc(c2O1)c3nnn(c4ccc(F)cc4)c3C(═O)NCCOc5ccc(C1)cc5C1
OC(═O)C1CCc2cccc(c2O1)n3nnc(c4cccc(c4)C(F)(F)F)c3C(═O)NCCOc5ccc(C1)cc5C1
Cc1ccc(cc1)c2nnc(c3ccc(C)c(OCC(═O)O)c3)n2C(═O)NCCOc4ccc(C1)cc4C1
OC(═O)Cn1ccc2cc(ccc12)c3nnc(c4ccc(F)cc4)n3C(═O)NCCOc5ccc(C1)cc5C1
COc1cccc(c1)c2nnc(c3ccc4CC(CC(═O)O)Cc4c3)n2C(═O)NCCOc5ccc(C1)cc5C1
OC(═O)CC1CCc2cc(ccc12)c3nnc(c4ccc(cc4)C(F)(F)F)n3C(═O)NCCOc5ccc(C1)cc5C1
COc1ccc(cc1)c2nnc(c3ccc4CCN(CC(═O)O)c4c3)n2C(═O)NCCOc5ccc(C1)cc5C1
OC(═O)C1Cc2cccc(c2O1)c3nnc(c4ccc(cc4)C(F)(F)F)n3C(═O)NCCOc5ccc(C1)cc5C1
OC(═O)Cc1csc2ccc(cc12)c3nnc(c4cccc(c4)C(F)(F)F)n3C(═O)NCCOc5ccc(C1)cc5C1
OC(═O)CC1CCc2cc(ccc2O1)c3nnc(c4ccc(F)cc4)n3C(═O)NCCOc5ccc(C1)cc5C1
Cc1ccc(cc1OCC(═O)O)c2nsc(C(═O)NCCOc3ccc(C1)cc3C1)c2c4cccc(c4)C(F)(F)F
OC(═O)C1CCc2ccc(cc12)c3cc(C(═O)NCCOc4ccc(C1)cc4C1)c(s3)c5ccc(cc5)C(F)(F)F
COc1ccc(cc1)c2sc(cc2C(═O)NCCOc3ccc(C1)cc3C1)c4ccc5c(ccn5CC(═O)O)c4
Cc1ccc(cc1)c2cc(c3cccc4CC(CC(═O)O)Cc34)c(s2)C(═O)NCCOc5ccc(C1)cc5C1
OC(═O)C1CCc2ccc(cc2C1)c3csc(C(═O)NCCOc4ccc(C1)cc4C1)c3c5ccc(cc5)C(F)(F)F
COc1cccc(c1)c2coc(C(═O)NCCOc3ccc(C1)cc3C1)c2c4cccc5c(CC(═O)O)csc45
OC(═O)CC1Cc2cccc(c2O1)c3occ(C(═O)NCCOc4ccc(C1)cc4C1)c3c5ccc(F)cc5
COc1ccc(cc1)c2cn(cc2C(═O)NCCOc3ccc(C1)cc3C1)c4ccc(C)c(OCC(═O)O)c4
OC(═O)C1Cc2cccc(c2C1)c3ccc(C(═O)NCCOc4ccc(C1)cc4C1)n3c5cccc(c5)C(F)(F)F
Cc1ccc(cc1)c2c(c[nH]c2c3cccc4CC(Cc34)C(═O)O)C(═O)NCCOc5ccc(C1)cc5C1
COc1ccc(cc1)c2cc(c3ccc4CC(Cc4c3)C(═O)O)n(c2)C(═O)NCCOc5ccc(C1)cc5C1
OC(═O)Cn1ccc2cc(ccc12)n3cc(cc3C(═O)NCCOc4ccc(C1)cc4C1)c5cccc(c5)C(F)(F)F
OC(═O)CC1Cc2cccc(c2C1)c3cn(cc3C(═O)NCCOc4ccc(C1)cc4C1)c5ccc(cc5)C(F)(F)F
Cc1ccc(cc1)c2cn(cc2c3cccc4CC(CC(═O)O)Cc34)C(═O)NCCOc5ccc(C1)cc5C1
COc1ccc(cc1)c2c(c[nH]c2C(═O)NCCOc3ccc(C1)cc3C1)c4ccc5C(CC(═O)O)CCc5c4
COc1cccc(c1)c2[nH]c(cc2C(═O)NCCOc3ccc(C1)cc3C1)c4ccc5CCN(CC(═O)O)c5c4
Cc1ccc(cc1)c2cc(cn2c3ccc4CCN(CC(═O)O)c4c3)C(═O)NCCOc5ccc(C1)cc5C1
OC(═O)Cc1csc2c(cccc12)c3cc(C(═O)NCCOc4ccc(C1)cc4C1)c([nH]3)c5ccc(F)cc5
OC(═O)C1Cc2cccc(c2O1)c3cn(cc3c4ccc(cc4)C(F)(F)F)C(═O)NCCOc5ccc(C1)cc5C1
Cc1ccc(cc1)c2cc(C(═O)NCCOc3ccc(C1)cc3C1)n(c2)c4cccc5CCC(Oc45)C(═O)O
OC(═O)Cc1csc2ccc(cc12)c3cc(cn3c4ccc(cc4)C(F)(F)F)C(═O)NCCOc5ccc(C1)cc5C1
OC(═O)C1Cc2cc(ccc2O1)c3cc(c4ccc(C1)cc4)c([nH]3)C(═O)NCCOc5ccc(C1)cc5C1
Cc1ccc(cc1)c2ccn(C(═O)NCCOc3ccc(C1)cc3C1)c2c4ccc5OC(CC(═O)O)Cc5c4
OC(═O)C1CCc2cccc(c2C1)n3ccc(C(═O)NCCOc4ccc(C1)cc4C1)c3c5ccc(C1)cc5
COc1cccc(c1)c2cc(cn2c3ccc4OC(CC(═O)O)CCc4c3)C(═O)NCCOc5ccc(C1)cc5C1
COc1ccc(cc1)c2cc(c3ccc(C)c(OCC(═O)O)c3)n(n2)C(═O)NCCOc4ccc(C1)cc4C1
Cc1ccc(cc1)n2cc(C(═O)NCCOc3ccc(C1)cc3C1)c(n2)c4ccc(C)c(OCC(═O)O)c4
COc1ccc(cc1)c2cn(nc2C(═O)NCCOc3ccc(C1)cc3C1)c4cccc5CC(Cc45)C(═O)O
OC(═O)C1Cc2ccc(cc2C1)c3n[nH]c(C(═O)NCCOc4ccc(C1)cc4C1)c3c5ccc(F)cc5
OC(═O)CC1Cc2cccc(c2C1)c3c(cnn3C(═O)NCCOc4ccc(C1)cc4C1)c5cccc(c5)C(F)(F)F
Cc1ccc(cc1)c2c(cnn2c3ccc4C(CC(═O)O)CCc4c3)C(═O)NCCOc5ccc(C1)cc5C1
OC(═O)CN1CCc2ccc(cc12)n3nc(cc3c4ccc(cc4)C(F)(F)F)C(═O)NCCOc5ccc(C1)cc5C1
OC(═O)Cc1csc2c(cccc12)c3nn(cc3c4cccc(c4)C(F)(F)F)C(═O)NCCOc5ccc(C1)cc5C1
COc1cccc(c1)c2c(cnn2C(═O)NCCOc3ccc(C1)cc3C1)c4cccc5CC(Oc45)C(═O)O
OC(═O)C1CCc2cccc(c2O1)c3nn(cc3C(═O)NCCOc4ccc(C1)cc4C1)c5cccc(c5)C(F)(F)F
COc1cccc(c1)c2[nH]nc(C(═O)NCCOc3ccc(C1)cc3C1)c2c4cccc5CCC(Oc45)C(═O)O
OC(═O)Cc1csc2ccc(cc12)c3[nH]nc(C(═O)NCCOc4ccc(C1)cc4C1)c3c5ccc(C1)cc5
COc1cccc(c1)n2ncc(C(═O)NCCOc3ccc(C1)cc3C1)c2c4ccc5OC(Cc5c4)C(═O)O
Cc1ccc(cc1)n2nc(cc2C(═O)NCCOc3ccc(C1)cc3C1)c4ccc5OC(CC(═O)O)Cc5c4
OC(═O)C1CCc2cc(ccc2O1)c3cn(nc3c4ccc(F)cc4)C(═O)NCCOc5ccc(C1)cc5C1
OC(═O)CC1CCc2cccc(c2O1)n3cc(c4cccc(c4)C(F)(F)F)c(n3)C(═O)NCCOc5ccc(C1)cc5C1
Cc1ccc(cc1)c2nc([nH]c2c3ccc(C)c(OCC(═O)O)c3)C(═O)NCCOc4ccc(C1)cc4C1
COc1ccc(cc1)c2cn(c3ccc4CC(Cc4c3)C(═O)O)c(n2)C(═O)NCCOc5ccc(C1)cc5C1
Cc1ccc(cc1)n2c(cnc2c3ccc4CC(Cc4c3)C(═O)O)C(═O)NCCOc5ccc(C1)cc5C1
COc1cccc(c1)c2c(ncn2c3ccc4CCC(C(═O)O)c4c3)C(═O)NCCOc5ccc(C1)cc5C1
OC(═O)Cn1ccc2cc(ccc12)c3c(ncn3C(═O)NCCOc4ccc(C1)cc4C1)c5cccc(c5)C(F)(F)F
OC(═O)CC1CCc2cc(ccc12)c3c(ncn3C(═O)NCCOc4ccc(C1)cc4C1)c5ccc(F)cc5
OC(═O)CC1CCc2cc(ccc12)n3cc(nc3c4ccc(cc4)C(F)(F)F)C(═O)NCCOc5ccc(C1)cc5C1
Cc1ccc(cc1)c2nc(cn2C(═O)NCCOc3ccc(C1)cc3C1)c4ccc5CCN(CC(═O)O)c5c4
Cc1ccc(cc1)c2ncn(C(═O)NCCOc3ccc(C1)cc3C1)c2c4cccc5c(CC(═O)O)csc45
OC(═O)Cc1csc2c(cccc12)n3c(cnc3c4ccc(cc4)C(F)(F)F)C(═O)NCCOc5ccc(C1)cc5C1
COc1cccc(c1)n2cc(nc2c3cccc4CC(Oc34)C(═O)O)C(═O)NCCOc5ccc(C1)cc5C1
OC(═O)C1Cc2cc(ccc2O1)c3cnc(C(═O)NCCOc4ccc(C1)cc4C1)n3c5ccc(cc5)C(F)(F)F
COc1cccc(c1)c2nc(c3cccc4CCC(Cc34)C(═O)O)c([nH]2)C(═O)NCCOc5ccc(C1)cc5C1
COc1cccc(c1)c2ncc(C(═O)NCCOc3ccc(C1)cc3C1)n2c4cccc5CCC(CC(═O)O)Oc45
OC(═O)CC1CCc2cccc(c2O1)c3nc(C(═O)NCCOc4ccc(C1)cc4C1)c([nH]3)c5cccc(c5)C(F)(F)F
OC(═O)C1Cc2ccc(cc2C1)c3nn(nc3c4ccc(cc4)C(F)(F)F)C(═O)NCCOc5ccc(C1)cc5C1
Cc1ccc(cc1)n2nc(C(═O)NCCOc3ccc(C1)cc3C1)c(n2)c4ccc5c(ccn5CC(═O)O)c4
OC(═O)CC1Cc2ccc(cc2C1)c3nn(nc3C(═O)NCCOc4ccc(C1)cc4C1)c5ccc(cc5)C(F)(F)F
COc1cccc(c1)c2nn(nc2C(═O)NCCOc3ccc(C1)cc3C1)c4cccc5CC(Oc45)C(═O)O
Cc1ccc(cc1)c2nn(nc2c3cccc4CCC(CC(═O)O)Oc34)C(═O)NCCOc5ccc(C1)cc5C1
Cc1ccc(cc1)c2c(nnn2C(═O)NCCOc3ccc(C1)cc3C1)c4ccc5CCC(C(═O)O)c5c4
Cc1ccc(cc1)c2c(nnn2c3cccc4c(CC(═O)O)csc34)C(═O)NCCOc5ccc(C1)cc5C1
COc1ccc(cc1)c2nnn(C(═O)NCCOc3ccc(C1)cc3C1)c2c4ccc5scc(CC(═O)O)c5c4
COc1cccc(c1)n2nnc(C(═O)NCCOc3ccc(C1)cc3C1)c2c4cccc5CCC(CC(═O)O)Oc45
Cc1ccc(cc1OCC(═O)O)n2nc(nc2C(═O)NCCOc3ccc(C1)cc3C1)c4ccc(cc4)C(F)(F)F
Cc1ccc(cc1)c2nc(nn2C(═O)NCCOc3ccc(C1)cc3C1)c4ccc5c(ccn5CC(═O)O)c4
OC(═O)Cn1ccc2cc(ccc12)n3nc(nc3c4ccc(F)cc4)C(═O)NCCOc5ccc(C1)cc5C1
COc1cccc(c1)c2nc(nn2c3ccc4CC(CC(═O)O)Cc4c3)C(═O)NCCOc5ccc(C1)cc5C1
COc1cccc(c1)c2nc(C(═O)NCCOc3ccc(C1)cc3C1)n(n2)c4ccc5scc(CC(═O)O)c5c4
OC(═O)CC1Cc2cc(ccc2O1)c3nc(nn3C(═O)NCCOc4ccc(C1)cc4C1)c5cccc(c5)C(F)(F)F
Cc1ccc(cc1OCC(═O)O)c2cnc(C(═O)NCCOc3ccc(C1)cc3C1)c(c2)c4ccc(F)cc4
COc1ccc(cc1)c2nccc(c3ccc4CC(CC(═O)O)Cc4c3)c2C(═O)NCCOc5ccc(C1)cc5C1
OC(═O)CN1CCc2ccc(cc12)c3cc(c4cccc(c4)C(F)(F)F)c(cn3)C(═O)NCCOc5ccc(C1)cc5C1
OC(═O)C1Cc2cccc(c2O1)c3ccc(nc3C(═O)NCCOc4ccc(C1)cc4C1)c5ccc(cc5)C(F)(F)F
COc1ccc(cc1)c2cncc(C(═O)NCCOc3ccc(C1)cc3C1)c2c4ccc5OC(CCc5c4)C(═O)O
Cc1ccc(cc1)c2ccc(C(═O)NCCOc3ccc(C1)cc3C1)c(n2)c4ccc5OC(CC(═O)O)CCc5c4
Cc1ccc(cc1)c2cnnc(C(═O)NCCOc3ccc(C1)cc3C1)c2c4ccc5CCN(CC(═O)O)c5c4
Cc1ccc(cc1)c2nnc(cc2C(═O)NCCOc3ccc(C1)cc3C1)c4cccc5CC(Oc45)C(═O)O
COc1cccc(c1)c2nncc(c3cccc4CCC(Cc34)C(═O)O)c2C(═O)NCCOc5ccc(C1)cc5C1
OC(═O)C1Cc2cccc(c2C1)c3ncc(C(═O)NCCOc4ccc(C1)cc4C1)c(n3)c5ccc(cc5)C(F)(F)F
OC(═O)Cn1ccc2cc(ccc12)c3ncc(c4ccc(F)cc4)c(n3)C(═O)NCCOc5ccc(C1)cc5C1
OC(═O)C1CCc2cccc(c2O1)c3cc(nc(n3)c4ccc(C1)cc4)C(═O)NCCOc5ccc(C1)cc5C1
Cc1ccc(cc1)c2nc(ncc2c3cccc4CCC(Oc34)C(═O)O)C(═O)NCCOc5ccc(C1)cc5C1
Cc1ccc(cc1)c2cnc(nc2C(═O)NCCOc3ccc(C1)cc3C1)c4ccc5OC(Cc5c4)C(═O)O
COc1ccc(cc1)c2c(ncnc2c3ccc4OC(CC(═O)O)Cc4c3)C(═O)NCCOc5ccc(C1)cc5C1
Cc1ccc(cc1)c2nnc(nc2C(═O)NCCOc3ccc(C1)cc3C1)c4ccc5CCC(C(═O)O)c5c4
OC(═O)CN1CCc2ccc(cc12)c3nc(nnc3c4ccc(F)cc4)C(═O)NCCOc5ccc(C1)cc5C1
Cc1ccc(cc1)c2cccc(c3ccc(C)c(OCC(═O)O)c3)c2C(═O)NCCOc4ccc(C1)cc4C1
COc1cccc(c1)c2c(cccc2c3cccc4CC(Cc34)C(═O)O)C(═O)NCCOc5ccc(C1)cc5C1
OC(═O)C1Cc2ccc(cc2C1)c3c(cccc3c4ccc(cc4)C(F)(F)F)C(═O)NCCOc5ccc(C1)cc5C1
COc1cccc(c1)c2ccc(C(═O)NCCOc3ccc(C1)cc3C1)c(c2)c4ccc5CCC(C(═O)O)c5c4
Cc1ccc(cc1)c2ccc(C(═O)NCCOc3ccc(C1)cc3C1)c(c2)c4ccc5CC(CC(═O)O)Cc5c4
OC(═O)CC1CCc2cc(ccc12)c3ccc(cc3c4cccc(c4)C(F)(F)F)C(═O)NCCOc5ccc(C1)cc5C1
OC(═O)CN1CCc2ccc(cc12)c3cc(cc(c3)c4ccc(C1)cc4)C(═O)NCCOc5ccc(C1)cc5C1
COc1ccc(cc1)c2cccc(C(═O)NCCOc3ccc(C1)cc3C1)c2c4cccc5CC(Oc45)C(═O)O
OC(═O)C1CCc2cccc(c2O1)c3ccc(C(═O)NCCOc4ccc(C1)cc4C1)c(c3)c5cccc(c5)C(F)(F)F
OC(═O)CC1Cc2cc(ccc2O1)c3c(cccc3c4ccc(C1)cc4)C(═O)NCCOc5ccc(C1)cc5C1
OC(═O)C1Cc2cccc(c2C1)n3c(nnc3c4cccc(c4)C(F)(F)F)C(═O)NCCOc5ccc(C1)cc5C1
OC(═O)C1CCc2cc(ccc2O1)n3c(nnc3c4ccc(F)cc4)C(═O)NCCOc5ccc(C1)cc5C1
Cc1ccc(cc1)c2c(snc2c3cccc(CC(═O)O)c3)C(═O)NCCOc4ccc(C1)cc4C1
COc1cccc(c1)c2cc(sc2C(═O)NCCOc3ccc(C1)cc3C1)c4ccc(CC(═O)O)cc4C1
Cc1ccc(cc1)c2c(csc2c3ccc(CC(═O)O)cc3C1)C(═O)NCCOc4ccc(C1)cc4C1
COc1cccc(c1)c2cc(c3cccc(CC(═O)O)c3)n(c2)C(═O)NCCOc4ccc(C1)cc4C1
COc1ccc(cc1)c2cc([nH]c2C(═O)NCCOc3ccc(C1)cc3C1)c4cccc(CC(═O)O)c4
OC(═O)Cc1cccc(c1)c2[nH]cc(C(═O)NCCOc3ccc(C1)cc3C1)c2c4cccc(c4)C(F)(F)F
Cc1ccc(cc1)n2cc(cc2c3cccc(CC(═O)O)c3)C(═O)NCCOc4ccc(C1)cc4C1
OC(═O)Cc1cccc(c1)c2ccn(C(═O)NCCOc3ccc(C1)cc3C1)c2c4cccc(c4)C(F)(F)F
Cc1ccc(cc1)c2cn(cc2c3cccc(CC(═O)O)c3)C(═O)NCCOc4ccc(C1)cc4C1
OC(═O)Cc1ccc(c(C1)c1)c2ccc(C(═O)NCCOc3ccc(C1)cc3C1)n2c4cccc(c4)C(F)(F)F
OC(═O)Cc1ccc(c(C1)c1)c2cc(C(═O)NCCOc3ccc(C1)cc3C1)c([nH]2)c4ccc(F)cc4
OC(═O)Cc1ccc(c(C1)c1)c2[nH]c(cc2c3ccc(F)cc3)C(═O)NCCOc4ccc(C1)cc4C1
OC(═O)Cc1ccc(c(C1)c1)c2c(ccn2c3ccc(cc3)C(F)(F)F)C(═O)NCCOc4ccc(C1)cc4C1
OC(═O)Cc1ccc(c(C1)c1)c2cc(cn2C(═O)NCCOc3ccc(C1)cc3C1)c4ccc(cc4)C(F)(F)F
COc1cccc(c1)c2c(ccn2c3ccc(CC(═O)O)cc3C1)C(═O)NCCOc4ccc(C1)cc4C1
COc1ccc(cc1)c2ccc(C(═O)NCCOc3ccc(C1)cc3C1)n2c4ccc(CC(═O)O)cc4C1
OC(═O)Cc1ccc(c(C1)c1)n2cc(C(═O)NCCOc3ccc(C1)cc3C1)c(c2)c4ccc(cc4)C(F)(F)F
Cc1ccc(cc1)c2cc(C(═O)NCCOc3ccc(C1)cc3C1)n(c2)c4ccc(CC(═O)O)cc4C1
COc1cccc(c1)n2cc(C(═O)NCCOc3ccc(C1)cc3C1)c(c2)c4ccc(CC(═O)O)cc4C1
COc1ccc(cc1)c2c(ccn2C(═O)NCCOc3ccc(C1)cc3C1)c4ccc(CC(═O)O)cc4C1
COc1ccc(cc1)c2[nH]cc(C(═O)NCCOc3ccc(C1)cc3C1)c2c4ccc(CC(═O)O)cc4C1
OC(═O)Cc1ccc(c(C1)c1)c2c[nH]c(C(═O)NCCOc3ccc(C1)cc3C1)c2c4cccc(c4)C(F)(F)F
OC(═O)Cc1cccc(c1)c2cc(nn2c3cccc(c3)C(F)(F)F)C(═O)NCCOc4ccc(C1)cc4C1
COc1cccc(c1)c2nn(cc2C(═O)NCCOc3ccc(C1)cc3C1)c4cccc(CC(═O)O)c4
COc1cccc(c1)c2n[nH]c(C(═O)NCCOc3ccc(C1)cc3C1)c2c4cccc(CC(═O)O)c4
OC(═O)Cc1cccc(c1)c2c(n[nH]c2c3cccc(c3)C(F)(F)F)C(═O)NCCOc4ccc(C1)cc4C1
OC(═O)Cc1cccc(c1)c2cnn(C(═O)NCCOc3ccc(C1)cc3C1)c2c4ccc(cc4)C(F)(F)F
COc1cccc(c1)n2ncc(C(═O)NCCOc3ccc(C1)cc3C1)c2c4ccc(CC(═O)O)cc4C1
COc1cccc(c1)c2cc(c3ccc(CC(═O)O)cc3C1)n(n2)C(═O)NCCOc4ccc(C1)cc4C1
Cc1ccc(cc1)n2nc(cc2c3ccc(CC(═O)O)cc3C1)C(═O)NCCOc4ccc(C1)cc4C1
OC(═O)Cc1ccc(c(C1)c1)n2ncc(C(═O)NCCOc3ccc(C1)cc3C1)c2c4ccc(cc4)C(F)(F)F
Cc1ccc(cc1)c2cc(C(═O)NCCOc3ccc(C1)cc3C1)n(n2)c4ccc(CC(═O)O)cc4C1
OC(═O)Cc1ccc(c(C1)c1)c2n[nH]c(C(═O)NCCOc3ccc(C1)cc3C1)c2c4ccc(cc4)C(F)(F)F
COc1ccc(cc1)c2[nH]nc(C(═O)NCCOc3ccc(C1)cc3C1)c2c4ccc(CC(═O)O)cc4C1
OC(═O)Cc1ccc(c(C1)c1)c2cn(nc2C(═O)NCCOc3ccc(C1)cc3C1)c4ccc(F)cc4
Cc1ccc(cc1)c2nn(cc2c3ccc(CC(═O)O)cc3C1)C(═O)NCCOc4ccc(C1)cc4C1
COc1cccc(c1)c2nc([nH]c2c3cccc(CC(═O)O)c3)C(═O)NCCOc4ccc(C1)cc4C1
OC(═O)Cc1cccc(c1)n2c(cnc2C(═O)NCCOc3ccc(C1)cc3C1)c4cccc(c4)C(F)(F)F
OC(═O)Cc1cccc(c1)n2c(cnc2c3ccc(C1)cc3)C(═O)NCCOc4ccc(C1)cc4C1
OC(═O)Cc1cccc(c1)c2nc(cn2C(═O)NCCOc3ccc(C1)cc3C1)c4ccc(F)cc4
COc1cccc(c1)n2c(cnc2C(═O)NCCOc3ccc(C1)cc3C1)c4ccc(CC(═O)O)cc4C1
OC(═O)Cc1ccc(c(C1)c1)c2[nH]c(nc2C(═O)NCCOc3ccc(C1)cc3C1)c4cccc(c4)C(F)(F)F
Cc1ccc(cc1)n2cnc(C(═O)NCCOc3ccc(C1)cc3C1)c2c4ccc(CC(═O)O)cc4C1
Cc1ccc(cc1)c2ncn(C(═O)NCCOc3ccc(C1)cc3C1)c2c4ccc(CC(═O)O)cc4C1
OC(═O)Cc1ccc(c(C1)c1)n2cnc(C(═O)NCCOc3ccc(C1)cc3C1)c2c4cccc(c4)C(F)(F)F
OC(═O)Cc1ccc(c(C1)c1)n2cc(nc2c3ccc(cc3)C(F)(F)F)C(═O)NCCOc4ccc(C1)cc4C1
Cc1ccc(cc1)c2cn(c3ccc(CC(═O)O)cc3C1)c(n2)C(═O)NCCOc4ccc(C1)cc4C1
COc1cccc(c1)n2cc(nc2c3ccc(CC(═O)O)cc3C1)C(═O)NCCOc4ccc(C1)cc4C1
Cc1ccc(cc1)c2nc([nH]c2C(═O)NCCOc3ccc(C1)cc3C1)c4ccc(CC(═O)O)cc4C1
COc1cccc(c1)c2nn(nc2C(═O)NCCOc3ccc(C1)cc3C1)c4ccc(CC(═O)O)cc4C1
OC(═O)Cc1ccc(c(C1)c1)c2nn(nc2c3ccc(F)cc3)C(═O)NCCOc4ccc(C1)cc4C1
OC(═O)Cc1ccc(c(C1)c1)c2nn(nc2C(═O)NCCOc3ccc(C1)cc3C1)c4ccc(cc4)C(F)(F)F
Cc1ccc(cc1)c2nnn(C(═O)NCCOc3ccc(C1)cc3C1)c2c4cccc(CC(═O)O)c4
OC(═O)Cc1ccc(c(C1)c1)n2nnc(C(═O)NCCOc3ccc(C1)cc3C1)c2c4ccc(cc4)C(F)(F)F
COc1cccc(c1)n2nnc(C(═O)NCCOc3ccc(C1)cc3C1)c2c4ccc(CC(═O)O)cc4C1
COc1cccc(c1)n2nc(nc2C(═O)NCCOc3ccc(C1)cc3C1)c4cccc(CC(═O)O)c4
Cc1ccc(cc1)c2nc(nn2C(═O)NCCOc3ccc(C1)cc3C1)c4ccc(CC(═O)O)cc4C1
OC(═O)Cc1ccc(c(C1)c1)c2nc(nn2c3ccc(cc3)C(F)(F)F)C(═O)NCCOc4ccc(C1)cc4C1
COc1cccc(c1)c2nc(nn2c3ccc(CC(═O)O)cc3C1)C(═O)NCCOc4ccc(C1)cc4C1
OC(═O)Cc1cccc(c1)c2nc(ccc2c3cccc(c3)C(F)(F)F)C(═O)NCCOc4ccc(C1)cc4C1
OC(═O)Cc1cccc(c1)c2ccc(C(═O)NCCOc3ccc(C1)cc3C1)c(n2)c4ccc(F)cc4
COc1cccc(c1)c2c(cncc2c3cccc(CC(═O)O)c3)C(═O)NCCOc4ccc(C1)cc4C1
COc1ccc(cc1)c2nccc(C(═O)NCCOc3ccc(C1)cc3C1)c2c4cccc(CC(═O)O)c4
OC(═O)Cc1cccc(c1)c2cc(cnc2C(═O)NCCOc3ccc(C1)cc3C1)c4ccc(F)cc4
OC(═O)Cc1ccc(c(C1)c1)c2ncc(cc2c3cccc(c3)C(F)(F)F)C(═O)NCCOc4ccc(C1)cc4C1
COc1ccc(cc1)c2ncc(cc2C(═O)NCCOc3ccc(C1)cc3C1)c4ccc(CC(═O)O)cc4C1
COc1ccc(cc1)c2nc(ccc2c3ccc(CC(═O)O)cc3C1)C(═O)NCCOc4ccc(C1)cc4C1
OC(═O)Cc1ccc(c(C1)c1)c2cnc(cc2C(═O)NCCOc3ccc(C1)cc3C1)c4cccc(c4)C(F)(F)F
OC(═O)Cc1ccc(c(C1)c1)c2cncc(C(═O)NCCOc3ccc(C1)cc3C1)c2c4ccc(cc4)C(F)(F)F
Cc1ccc(cc1)c2cc(cc(n2)C(═O)NCCOc3ccc(C1)cc3C1)c4ccc(CC(═O)O)cc4C1
Cc1ccc(cc1)c2c(ccnc2C(═O)NCCOc3ccc(C1)cc3C1)c4ccc(CC(═O)O)cc4C1
COc1cccc(c1)c2nnc(cc2C(═O)NCCOc3ccc(C1)cc3C1)c4cccc(CC(═O)O)c4
COc1cccc(c1)c2c(cnnc2c3ccc(CC(═O)O)cc3C1)C(═O)NCCOc4ccc(C1)cc4C1
COc1cccc(c1)c2nnc(cc2c3ccc(CC(═O)O)cc3C1)C(═O)NCCOc4ccc(C1)cc4C1
COc1cccc(c1)c2c(cnnc2C(═O)NCCOc3ccc(C1)cc3C1)c4ccc(CC(═O)O)cc4C1
OC(═O)Cc1ccc(c(C1)c1)c2cnnc(c3ccc(C1)cc3)c2C(═O)NCCOc4ccc(C1)cc4C1
OC(═O)Cc1ccc(c(C1)c1)c2cc(nnc2C(═O)NCCOc3ccc(C1)cc3C1)c4ccc(cc4)C(F)(F)F
COc1cccc(c1)c2nc(cnc2C(═O)NCCOc3ccc(C1)cc3C1)c4cccc(CC(═O)O)c4
Cc1ccc(cc1)c2ncc(nc2c3cccc(CC(═O)O)c3)C(═O)NCCOc4ccc(C1)cc4C1
OC(═O)Cc1ccc(c(C1)c1)c2ncc(nc2C(═O)NCCOc3ccc(C1)cc3C1)c4ccc(F)cc4
COc1ccc(cc1)c2nc(cc(n2)c3cccc(CC(═O)O)c3)C(═O)NCCOc4ccc(C1)cc4C1
OC(═O)Cc1cccc(c1)c2nc(ncc2C(═O)NCCOc3ccc(C1)cc3C1)c4cccc(c4)C(F)(F)F
Cc1ccc(cc1)c2cc(nc(n2)C(═O)NCCOc3ccc(C1)cc3C1)c4cccc(CC(═O)O)c4
COc1ccc(cc1)c2ncc(C(═O)NCCOc3ccc(C1)cc3C1)c(n2)c4ccc(CC(═O)O)cc4C1
COc1ccc(cc1)c2cnc(nc2c3ccc(CC(═O)O)cc3C1)C(═O)NCCOc4ccc(C1)cc4C1
OC(═O)Cc1ccc(c(C1)c1)c2ncnc(c3ccc(cc3)C(F)(F)F)c2C(═O)NCCOc4ccc(C1)cc4C1
OC(═O)Cc1ccc(c(C1)c1)c2cnc(nc2C(═O)NCCOc3ccc(C1)cc3C1)c4ccc(F)cc4
OC(═O)Cc1ccc(c(C1)c1)c2c(ncnc2c3ccc(C1)cc3)C(═O)NCCOc4ccc(C1)cc4C1
COc1ccc(cc1)c2nnc(c3ccc(CC(═O)O)cc3C1)c(n2)C(═O)NCCOc4ccc(C1)cc4C1
OC(═O)Cc1ccc(c(C1)c1)c2nnc(nc2c3cccc(c3)C(F)(F)F)C(═O)NCCOc4ccc(C1)cc4C1
OC(═O)Cc1ccc(c(C1)c1)c2nnc(C(═O)NCCOc3ccc(C1)cc3C1)c(n2)c4cccc(c4)C(F)(F)F
COc1ccc(cc1)c2cccc(c3cccc(CC(═O)O)c3)c2C(═O)NCCOc4ccc(C1)cc4C1
OC(═O)Cc1cccc(c1)c2cccc(C(═O)NCCOc3ccc(C1)cc3C1)c2c4cccc(c4)C(F)(F)F
OC(═O)Cc1cccc(c1)c2cc(cc(c2)c3ccc(C1)cc3)C(═O)NCCOc4ccc(C1)cc4C1
Cc1ccc(cc1)c2c(cccc2c3cccc(CC(═O)O)c3)C(═O)NCCOc4ccc(C1)cc4C1
COc1ccc(cc1)c2cc(ccc2c3ccc(CC(═O)O)cc3C1)C(═O)NCCOc4ccc(C1)cc4C1
OC(═O)Cc1ccc(c(C1)c1)c2cc(ccc2C(═O)NCCOc3ccc(C1)cc3C1)c4ccc(C1)cc4
OC(═O)Cc1ccc(c(C1)c1)c2cc(ccc2C(═O)NCCOc3ccc(C1)cc3C1)c4ccc(cc4)C(F)(F)F
OC(═O)Cc1ccc(c(C1)c1)n2c(nnc2c3cccc(c3)C(F)(F)F)C(═O)NCCOc4ccc(C1)cc4C1
COc1ccc(cc1)n2c(nnc2c3ccc(CC(═O)O)cc3C1)C(═O)NCCOc4ccc(C1)cc4C1

The activity of the compounds of the present invention as PPAR modulators may be illustrated by using the following assay. The compounds listed above, which have not yet been made and/or tested, are predicted to have activity in this assay.

### Biological Activity Assay

Transcriptional Assay

Transfection assays are used to assess the ability of compounds of the invention to modulate the transcriptional activity of the PPARs. Briefly, expression vectors for chimeric proteins containing the DNA binding domain of yeast GAL4 fused to the ligand-binding domain (LED) of either PPARα, PPARγ or PPARδ are introduced via transient transfection into mammalian cells, together with a reporter plasmid where the luciferase gene is under the control of a GAL4 binding site. Upon exposure to a PPAR modulator, PPAR transcriptional activity varies, and this can be monitored by changes in luciferase levels. If transfected cells are exposed to a PPAR agonist, PPAR-dependent transcriptional activity increases and luciferase levels rise.

293T human embryonic kidney cells (8×106) are seeded in a 175 cm2 flask a day prior to me start of the experiment in 10% FBS, 1% Penicillin/Streptomycin/Fungizome, DMEM Media. The cells are harvested by washing with PBS (30 ml) and then dissociating using trypsin (0.05%; 3 ml). The trypsin is inactivated by the addition of assay media (DMEM, CA-dextran fetal bovine serum (5%). The cells are spun down and resuspended to 170,000 cells/ml. A Transfection mixture of GAL4-PPAR LBD expression plasmid (1 μg), UAS-luciferase reporter plasmid (1 μg), Fugene (3:1 ratio; 6 μL) and serum-free media (200 mL) was prepared and incubated for 15-40 minutes at room temperature. Transfection mixtures are added to the cells to give 0.16M cells/mL, and cells (50 μL/well) are then plated into 384 white, solid-bottom, TC-treated plates. The cells are further incubated at 37° C., 5.0% C02 for 5-7 hours. A 12-point series of dilutions (3 fold serial dilutions) are prepared for each test compound in DMSO with a starting compound concentration of 10 μM. Test compound (500 nl) is added to each well of cells in the assay plate and the cells are incubated at 37° C., 5.0% CO₂ for 18-24 hours. The cell lysis/luciferase assay buffer, Bright-Glo™ (25%; 25 μL; Promega), is added to each well. After a further incubation for 5 minutes at room temperature, the luciferase activity is measured.

Raw luminescence values are normalized by dividing them by the value of the DMSO control present on each plate. Normalized data is analyzed and dose-response curves are fitted using Prizm graph fitting program. EC₅₀ is defined as the concentration at which the compound elicits a response that is halfway between the maximum and minimum values. Relative efficacy (or percent efficacy) is calculated by comparison of the response elicited by the compound with the maximum value obtained for a reference PPAR modulator.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula (I): 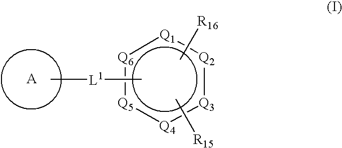
Or a salt, ester, or prodrug thereof, wherein:
A is selected from the group consisting of aryl, heteroaryl, cycloalkyl, and heterocycloalkyl, any of which may be optionally substituted;
L¹ is selected from the group consisting of —X—, —XOX—, —XS(O)₀₋₂X— and —XS(O)₀₋₂XO—, —XOXO—, —XN(R¹)X—, —XS(O)₂N(R¹)X—, —XC(O)N(R¹)X—, —X(CF₂)₁₋₃X—, —XC(═O)X—, —XC(═O)OX—, —XN(R¹)C(═O)X—, —XN(R¹)C(═O)N(R²)X—, —XN(R¹)SO₂N(R²)X—, —XN(R¹)C(═O)OX, —XP(═O)(OR¹)X—, —XP(═O)(NR¹)X—, —XP(═S)(OR¹)X—, —XP(═S)(NR¹)X—, and —XS(═O)(═NR¹)X—;
R¹ and R² are hydrogen or are independently selected from the group consisting of lower alkyl, lower alkoxy, lower perhaloalkyl, lower alkenyl, lower alkynyl, lower heteroalkyl, lower alkoxyalkyl, alkylamino, alkylaminoalkyl, alkylcarbonyl, amido, aminoalkyl, aryl, arylalkyl, arylalkenyl, arylalkynyl, lower cycloalkyl, lower cycloalkyl alkyl, lower haloalkyl, heteroaryl, heteroarylalkyl, heteroarylalkenyl, and heterocycloalkyl, any of which may be optionally substituted; or R¹ and R², together with the atoms to which they are attached, may be joined to form an optionally substituted heterocycloalkyl or optionally substituted cycloalkyl moiety;
X is a bond, or is selected from lower alkyl and lower alkenyl, either of which may be optionally substituted;
Q¹-Q⁶ are independently selected from the group consisting of a bond, C, NR³, S, and O;
R³ is hydrogen or is selected from the group consisting of lower alkyl, lower alkenyl, aryl, arylalkyl, lower cycloalkyl, lower cycloalkyl alkyl, lower haloalkyl, heteroaryl, heteroarylalkyl, heterocycloalkyl, R¹⁵, and R¹⁶, any of which may be optionally substituted;
R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted;
Y is selected from the group consisting of lower alkyl, lower alkenyl, lower alkynyl, —C(O)N(R¹)— and —OX—, any of which may be optionally substituted;
R¹⁸ is selected from the group consisting of —XOXOR⁵, —XOR⁵, —XC(O)OR⁵, —XC(O)NR⁴R⁴, —XC(O)NR⁴OR⁴, —XC(O)OXOR⁵, —XC(O)XOR⁵, —XC(O)N(R⁴)XOR⁵, —XC(O)N(R⁴)(R⁵) and —XC(O)N(R⁴)XR⁵; or R¹⁸ may be further selected from the group consisting of lower cycloalkyl, heterocycloalkyl, aryl and heteroaryl, any of which may be further optionally substituted with —XS(O)₀₋₂R¹, —XS(O)₀₋₂XR¹⁹, —XN(R¹)(R²), —XN(R¹)S(O)₀₋₂R¹, —XN(R¹)C(O)R², —XC(O)N(R¹)(R²), —XN(R¹)(R¹⁹), —XC(O)N(R¹)(R¹⁹), —XC(O)R¹⁹, —XC(O)OR¹, —XN(R¹)XR¹⁹ and —XOXR;
R⁴ and R⁵ are hydrogen or are independently selected from the group consisting of lower alkyl, lower cycloalkyl, heterocycloalkyl, aryl and heteroaryl, any of which may be optionally substituted; or R⁴ and R⁵, together with the atom to which they are attached, may be joined to form an optionally substituted heterocycloalkyl or optionally substituted heteroaryl moiety; and
R¹⁹ is selected from the group consisting of lower cycloalkyl, heterocycloalkyl, aryl and heteroaryl, any of which may be optionally substituted.

2. The compound as recited in claim 1:
wherein the cyclic moiety represented by the group: 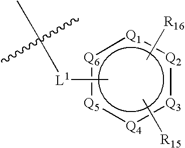
is independently selected from the heteroaromatic and aromatic groups consisting of:

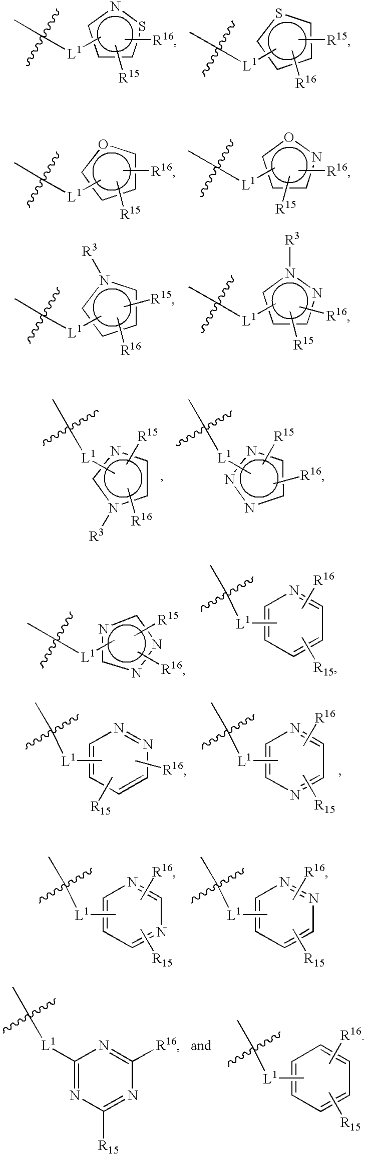

3. The compound as recited in claim 1:
wherein:
A has a structural formula selected from the group consisting of: 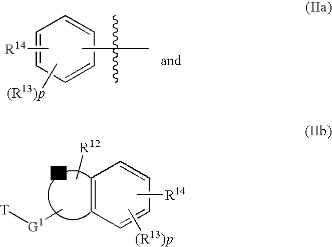
B is an optionally substituted five- to eight-membered carbocycle or heterocycle;
T is selected from the group consisting of hydrogen, —C(O)OH, —C(O)OR¹, —C(O)NH₂, —C(O)N(R¹)(R²), tetrazolyl-, and thiazolidine-2,4-dione-5-yl-;
G¹ is selected from the group consisting of —(CR¹R²)ₙ—, -Z(CR¹R²)ₙ—, —(CR¹R²)ₙZ -, and —(CR¹R²)ᵣZ(CR¹R²)ₛ—;
Z is O, S or NR³;
n is an integer from 0 to 2;
r and s are independently integers from 0 to 1;
R¹² is selected from the group consisting of hydrogen, halogen, hydroxy, optionally substituted lower alkyl optionally substituted lower cycloalkyl, optionally substituted heteroalkyl, optionally substituted cycloheteroalkyl, optionally substituted lower alkynyl, lower perhaloalkyl, lower perhaloalkoxy, optionally substituted lower alkoxy, nitro, cyano, and NH₂;
R¹³ is selected from hydrogen and the group consisting of halogen, lower alkyl lower alkoxy, lower halo alkyl lower haloalkoxy, aryl, heteroaryl, lower cycloalkyl and heterocycloalkyl, any of which may be optionally substituted;
p is an integer from 0 to 3; and
R¹⁴ is selected from the group consisting of hydrogen, —XOXC(O)OR¹, —XC(O)OH, —XC(O)OR¹, —XC(O)NH₂, —C(O)N(R¹)(R²), —X-tetrazole, and —X-[(thiazolidine-2,4-dione)-5-yl]-.

4. The compound as recited in claim 3 having the structural Formula III: 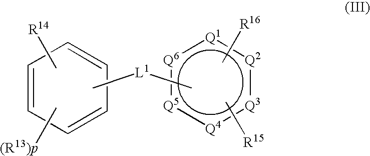
Or a salt, ester, or prodrug thereof, wherein:
L¹ is selected from the group consisting of —X—, —XOX—, —XS(O)₀₋₂X—, —XS(O)₀₋₂XO—, —XOXO—, —XS(O)₂N(R¹)X—, —XC(O)N(R¹)X—, —X(CF₂)₁₋₃X—, —XN(R¹)C(═O)X—, —XN(R¹)C(═O)N(R²)X—, —XN(R¹)SO₂N(R²)X—, and —XN(R¹)C(═O)OX—;
R¹ and R² are hydrogen or are independently selected from the group consisting of lower alkyl, lower alkoxy, lower alkenyl, lower alkynyl, lower heteroalkyl, lower alkoxyalkyl, alkylaminoalkyl, alkylcarbonyl, aryl, arylalkyl, lower cycloalkyl, lower cycloalkyl alkyl, lower haloalkyl, heteroaryl, heteroarylalkyl, and heterocycloalkyl, any of which may be optionally substituted; or R¹ and R², together with the atoms to which they are attached, may be joined to form an optionally substituted heterocycloalkyl or optionally substituted cycloalkyl moiety;
X is a bond, or is selected from lower alkyl and lower alkenyl, either of which may be optionally substituted;
Q¹-Q⁶ are independently selected from the group consisting of a bond, C, NR³, S, and O;
R¹³ is selected from hydrogen and from the group consisting of halogen, lower alkyl, lower alkoxy, lower haloalkyl, lower haloalkoxy, lower cycloalkyl and heterocycloalkyl, any of which may be optionally substituted;
p is an integer from 0 to 3;
R¹⁴ is selected from the group consisting of hydrogen, —XOXC(O)OR¹, —XC(O)OH, —XC(O)OR¹, —X-tetrazole, and —X-[(thiazolidine-2,4-dione)-5-yl]-;
R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted;
Y is selected from the group consisting of lower alkyl, lower alkenyl, lower alkynyl, —C(O)N(R¹)— and —OX—, any of which may be optionally substituted;
R¹⁸ is selected from the group consisting of —XOXOR⁵, —XOR⁵, —XC(O)OR⁵, —XC(O)NR⁴R⁴, —XC(O)NR⁴OR⁴, —XC(O)OXOR⁵, —XC(O)XOR⁵, —XC(O)N(R⁴)XOR⁵, —XC(O)N(R⁴)(R⁵) and —XC(O)N(R⁴)XR⁵; or R¹⁸ may be further selected from the group consisting of lower cycloalkyl, heterocycloalkyl, aryl and heteroaryl, any of which may be further optionally substituted with —XS(O)₀₋₂R¹, —XS(O)₀₋₂XR¹⁹, —XN(R¹)(R²), —XN(R¹)S(O)₀₋₂R¹, —XN(R¹)C(O)R², —XC(O)N(R¹)(R²), —XN(R¹)(R¹⁹), —XC(O)N(R¹)(R¹⁹), —XC(O)R¹⁹, —XC(O)OR¹, —XN(R¹)XR¹⁹ and —XOXR;
R⁴ and R⁵ are selected from hydrogen or are independently selected from a group consisting of lower alkyl, lower cycloalkyl, heterocycloalkyl, aryl and heteroaryl, any of which may be optionally substituted; or R⁴ and R⁵, together with the atom to which they are attached, may be joined to form an optionally substituted heterocycloalkyl or optionally substituted heteroaryl moiety; and
R¹⁹ is selected from the group consisting of lower cycloalkyl, heterocycloalkyl, aryl and heteroaryl, any of which may be optionally substituted.

5. The compound as recited in claim 3 having the structural Formula IV: 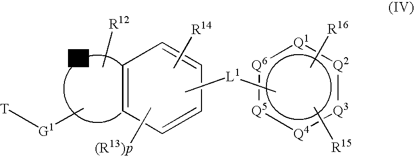
wherein:
B is an optionally substituted five- to eight-membered carbocycle or heterocycle;
T is selected from the group consisting of hydrogen, —C(O)OH, —C(O)OR¹, —C(O)NH₂, —C(O)N(R¹)(R²), tetrazolyl-, and thiazolidine-2,4-dione-5-yl-;
G¹ is selected from the group consisting of —(CR¹R²)ₙ—, -Z(CR¹R²)ₙ—, —(CR¹R²)ₙZ-, and —(CR¹R²)ᵣZ(CR¹R²)ₛ—;
R¹ and R² are hydrogen or are independently selected from the group consisting of lower alkyl, lower alkoxy, lower alkenyl, lower alkynyl, lower heteroalkyl, lower alkoxyalkyl, alkylaminoalkyl, alkylcarbonyl, aryl, arylalkyl, lower cycloalkyl, lower cycloalkyl alkyl, lower haloalkyl, heteroaryl, heteroarylalkyl, and heterocycloalkyl, any of which may be optionally substituted; or R¹ and R², together with the atoms to which they are attached, may be joined to form an optionally substituted heterocycloalkyl or optionally substituted cycloalkyl moiety;
n is an integer from 0 to 2;
r and s are independently integers from 0 to 1;
Z is O, S or NR³;
R³ is hydrogen or is selected from the group consisting of lower alkyl, lower alkenyl, aryl, arylalkyl, lower cycloalkyl, lower cycloalkyl alkyl, lower haloalkyl, heteroaryl, heteroarylalkyl, heterocycloalkyl, R¹⁵ and R¹⁶, any of which may be optionally substituted;
L¹ is selected from the group consisting of —X—, —XOX—, —XS(O)₀₋₂X—, —XS(O)₀₋₂XO—, —XOXO—, —XS(O)₂N(R¹)X—, —XC(O)N(R¹)X—, —X(CF₂)₁₋₃X—, —XN(R¹)C(═O)X—, —XN(R¹)C(═O)N(R²)X—, —XN(R¹)SO₂N(R²)X—, and —XN(R¹)C(═O)OX—;
X is a bond, or is selected from lower alkyl and lower alkenyl, either of which may be optionally substituted;
Q¹-Q⁶ are independently selected from the group consisting of a bond, C, NR³, S, and O;
R¹² is selected from the group consisting of hydrogen, halogen, optionally substituted lower alkyl, optionally substituted lower cycloalkyl, optionally substituted cycloheteroalkyl, lower perhaloalkyl, lower perhaloalkoxy, optionally substituted lower alkoxy, nitro, and cyano;
R¹³ is selected from hydrogen and the group consisting of halogen, lower alkyl, lower alkoxy, lower haloalkyl, lower haloalkoxy, lower cycloalkyl and heterocycloalkyl, any of which may be optionally substituted;
p is an integer from 0 to 3;
R¹⁴ is selected from the group consisting of hydrogen, —XOXC(O)OR¹, —XC(O)OH, —XC(O)OR¹, —X-tetrazole, and —X-[(thiazolidine-2,4-dione)-5-yl]-;
R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted;
Y is selected from the group consisting of lower alkyl, lower alkenyl, lower alkynyl, —C(O)N(R¹)— and —OX—, any of which may be optionally substituted;
R¹⁸ is selected from the group consisting of —XOXOR⁵, —XOR⁵, —XC(O)OR⁵, —XC(O)NR⁴R⁴, —XC(O)NR⁴OR⁴, —XC(O)OXOR⁵, —XC(O)XOR⁵, —XC(O)N(R⁴)XOR⁵, —XC(O)N(R⁴)(R⁵) and —XC(O)N(R⁴)XR⁵; or R¹⁸ may be further selected from the group consisting of lower cycloalkyl, heterocycloalkyl, aryl and heteroaryl, any of which may be further optionally substituted with —XS(O)₀₋₂R¹, —XS(O)₀₋₂XR¹⁹, —XN(R¹)(R²), —XN(R¹)S(O)₀₋₂R¹, —XN(R¹)C(O)R², —XC(O)N(R¹)(R²), —XN(R¹)(R¹⁹), —XC(O)N(R¹)(R¹⁹), —XC(O)R¹⁹, —XC(O)OR¹, —XN(R¹)XR¹⁹ and XOXR;
R⁴ and R⁵ are independently selected from hydrogen or are selected from a group consisting of lower alkyl, lower cycloalkyl, heterocycloalkyl, aryl and heteroaryl, any of which may be optionally substituted; or R⁴ and R⁵, together with the atom to which they are attached, may be joined to form an optionally substituted heterocycloalkyl or optionally substituted heteroaryl moiety; and
R¹⁹ is selected from the group consisting of lower cycloalkyl, heterocycloalkyl, aryl and heteroaryl, any of which may be optionally substituted.

6. The compound as recited in claim 4 wherein:
L¹ is selected from the group consisting of —X—, —XOX—, —XOXO—, —XS(O)₀₋₂X—, and —XS(O)₀₋₂XO—
X is a bond or optionally substituted alkyl;
R¹³ is selected from the group consisting of hydrogen, optionally substituted lower alkyl, optionally substituted lower alkoxy and halogen;
R¹⁴ is selected from the group consisting of hydrogen, —XOXC(O)OR¹ and —XC(O)OR¹;
R¹ is selected from hydrogen and optionally substituted lower alkyl; and
R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted.

7. The compound as recited in claim 6 having the structural Formula IIIa: 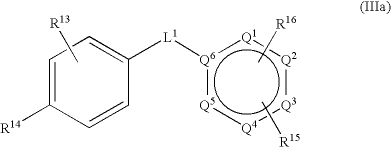
wherein:
L¹ is selected from the group consisting of —S(O)₀₋₂(CH₂)₁₋₄O—, —O(CH₂)₁₋₄S(O)₀₋₂—, —CH₂S(O)₀₋₂—, —S(O)₀₋₂CH₂—, —S(O)₀₋₂—, —O(CH₂)₁₋₄O—, —CH₂O— and —OCH₂—;
R¹³ is selected from is selected from the group consisting of hydrogen, optionally substituted lower alkyl, optionally substituted lower alkoxy and halogen;
R¹⁴ is selected from is selected from the group consisting of hydrogen, —OCH₂C(O)OH, —CH₂C(O)OH, —OC(CH₃)₂C(O)OH, —CH═CH—C(O)OH, —(CH₂)₂C(O)OH, and —OCH₂-tetrazolyl;
R¹⁵ and R¹⁶ are independently selected from —R¹⁸ and —YR¹⁸; or R¹⁵ and R¹⁶ together with the atoms to which R¹⁵ and R¹⁶ are attached form 4,5-dihydro-naphtho[1,2-d]naphthal-2-yl, 4H-chromeno[4,3-d]naphthal-2-yl, 5,6-dihydro-4H-3-thia-1-aza-benzo[e]azulen-2-yl, benzthiazolyl, benzoxazolyl and 1-oxa-3-aza-cyclopenta[a]naphthalene-2-yl, any of which may be optionally substituted;
Y is selected from the group consisting of optionally substituted lower alkyl, optionally substituted lower alkenyl, —C(O)NH— and —O(CH₂)₁₋₃—; and
R¹⁸ is selected from the group consisting of —XOR⁴, —XC(O)OR⁴, —XC(O)OXOR⁴, —XC(O)N(R⁴)XOR⁵, —XC(O)N(R⁴)(R⁵), —XC(O)N(R⁴)XR⁵, phenyl, biphenyl, cyclohexyl, naphthyl, benzo[1,3]dioxol-5-yl, benzo[b]furanyl, pyridinyl, pyrimidinyl, dibenzo-furan-2-yl, furanyl, benzo[b]thiophene, thiophenyl, phenoxathiin-4-yl, benzoxazolyl, 3-oxo-3,4-dihydro-2H-benzo[1,4]oxazin-6-yl, 2-oxo-2,3-dihydro-benzooxazol-6-yl, 2,3-dihydro-benzo[1,4]dioxin-6-yl, benzoxazolyl, 3,4-dihydro-2H-benzo[b][1,4]-dioxepin-7-yl and quinolinyl; any of which may be optionally substituted.

8. The compound as recited in claim 7 having structural Formula (IIa) selected from the group consisting of: 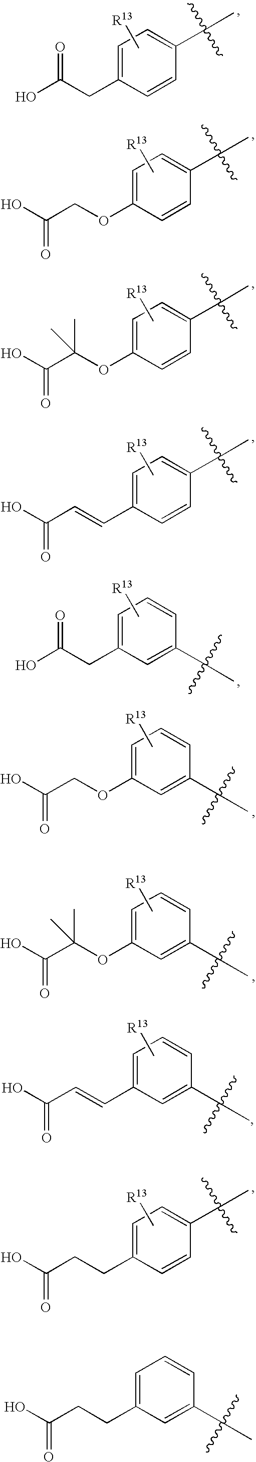 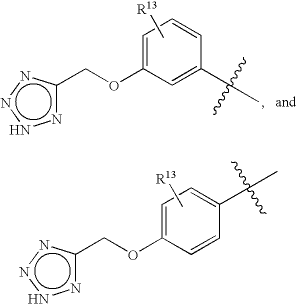
wherein:
R¹³ is selected from the group consisting of hydrogen, halogen, lower alkyl and lower alkoxy.

9. The compound as recited in claim 8 having the structural Formula V: 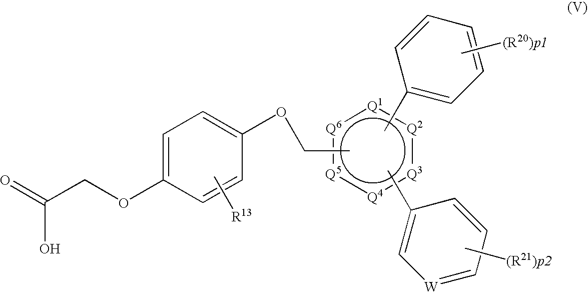
wherein:
W is selected from N and CH;
R¹³ is selected from the group consisting of hydrogen, optionally substituted lower alkyl optionally substituted lower alkoxy, and halogen;
R²⁰ is selected from trifluoromethyl and trifluoromethoxy;
R²¹ is selected from isopropyloxy and methoxy; and
p1 and p2 are independently selected from an integer from 0 to 2.

10. A compound as recited in claim 9 having the structural Formula selected from the group consisting of: 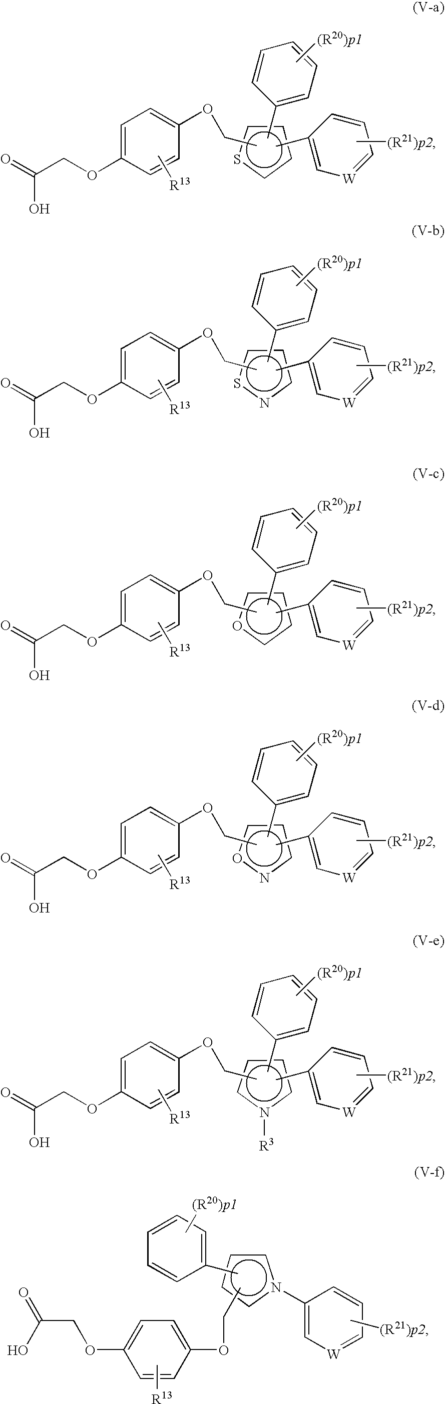 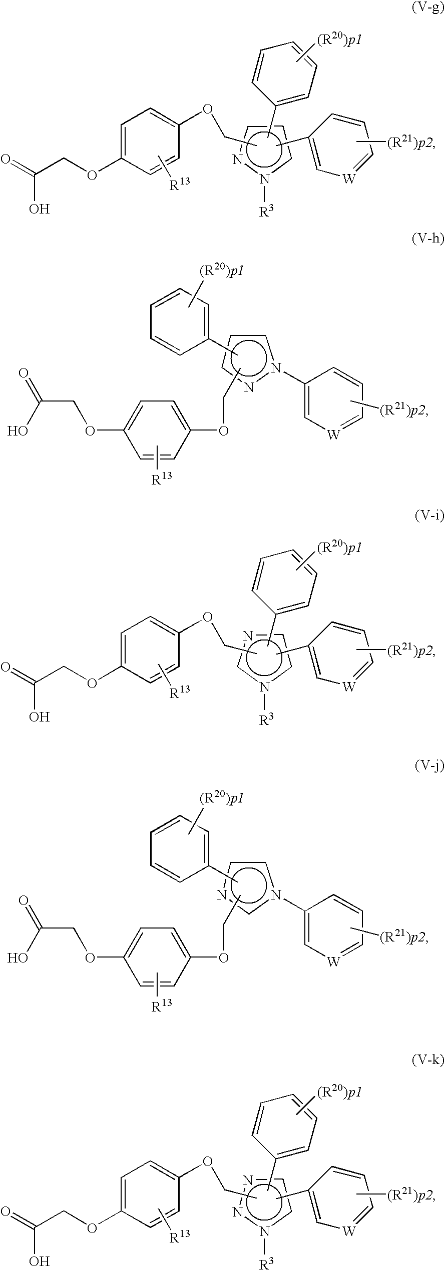 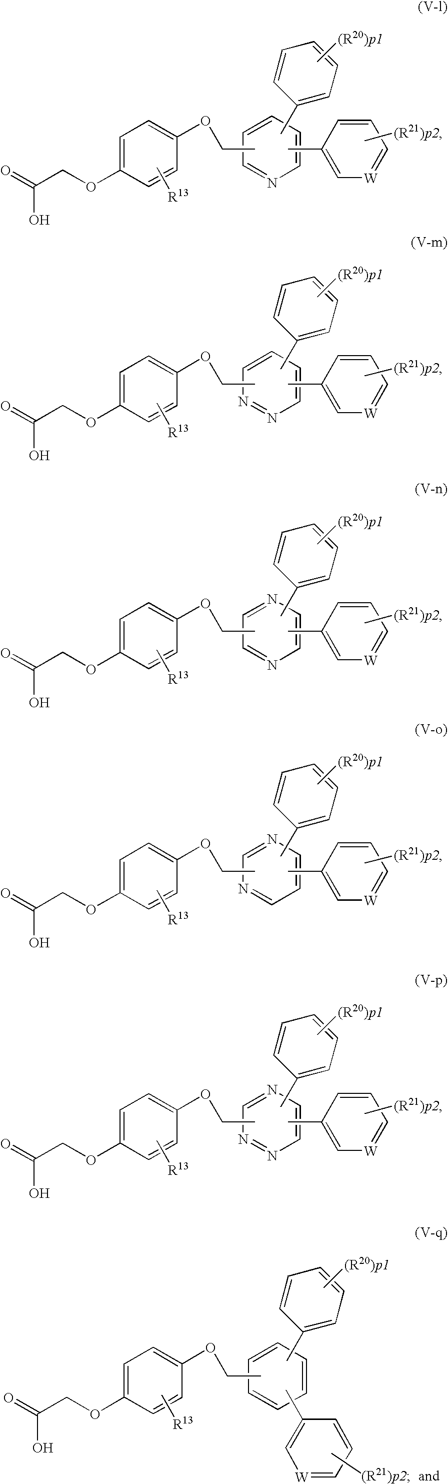
wherein the (R²⁰)ₚ₁-aryl and (R²¹)ₚ₂-(hetero)aryl moieties are attached to two contiguous central ring atoms.

11. The compound as recited in claim 6 having the structural Formula (VI): 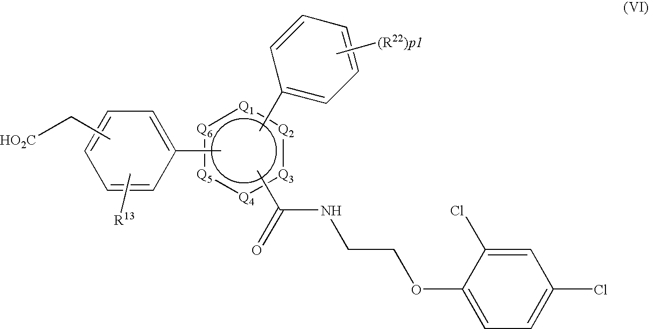
wherein:
R¹³ is selected from the group consisting of hydrogen, optionally substituted lower alkyl optionally substituted lower alkoxy and halogen;
R²² is selected from the group consisting of hydrogen, methoxy, isopropyloxy, trifluoromethoxy, fluoro, chloro, trifluoromethyl, methyl, and nitro; and
p1 is an integer from 0 to 2.

12. A compound as recited in claim 11 having the structural Formula selected from the group consisting of: 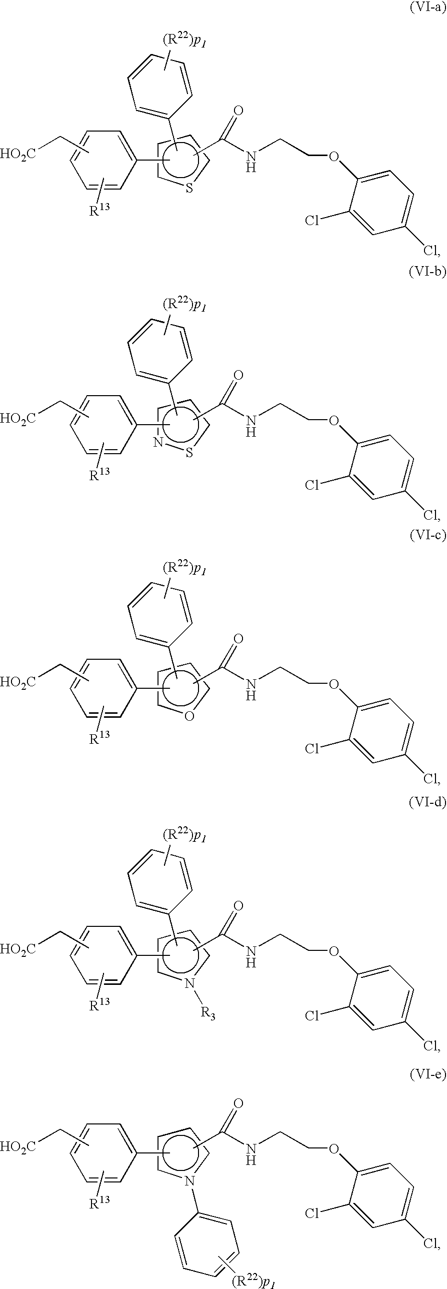 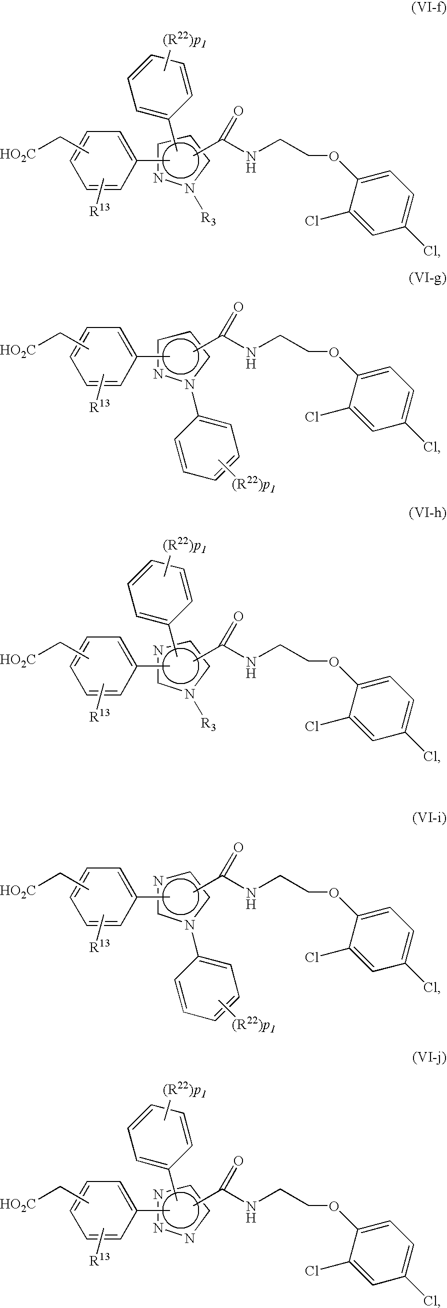 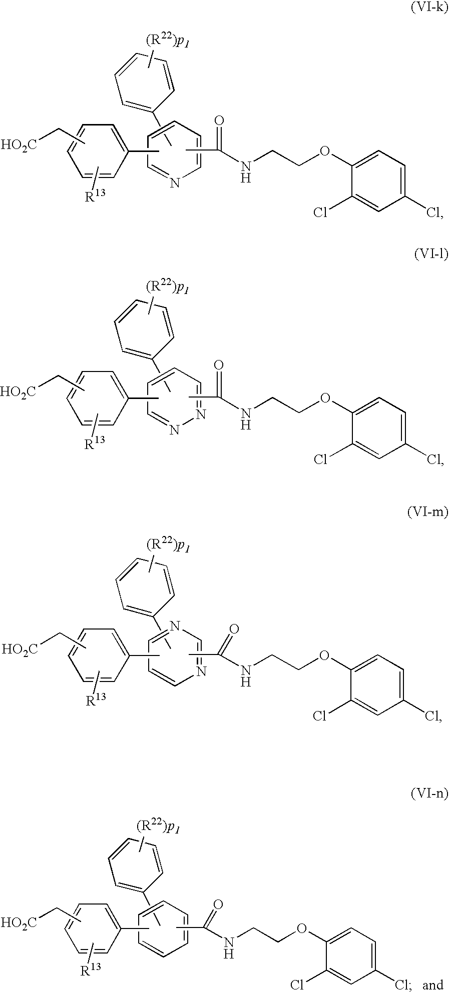
wherein the three substituents are attached to three contiguous ring atoms of the central ring moiety.

13. The compound as recited in claim 5 wherein:
T is selected from the group consisting of —C(O)OH, —C(O)OR¹, and tetrazolyl;
G¹ is selected from the group consisting of —(CR¹R²)ₙ—, —(CR¹R²)ₙZ-, and —(CR¹R²)ᵣZ(CR¹R²)ₛ—;
R¹ and R² are hydrogen or optionally substituted lower alkyl;
Z is O, S or NR³;
R³ is hydrogen or is selected from the group consisting of lower alkyl, R¹⁵, and R¹⁶, any of which may be optionally substituted;
L¹ is selected from the group consisting of —X—, —XOX—, —XS(O)₀₋₂X— and —XS(O)₀₋₂XO—;
R¹² is selected from the group consisting of hydrogen, halogen, optionally substituted lower alkyl, lower perhaloalkyl, lower perhaloalkoxy, optionally substituted lower alkoxy, nitro, and cyano;
R¹³ is selected from the group consisting of hydrogen, optionally substituted lower alkyl, optionally substituted lower alkoxy and halogen;
R¹⁴ is selected from the group consisting of hydrogen, —XOXC(O)OR¹ and XC(O)OR¹; and
R¹⁵ and R¹⁶ are independently selected from —R¹⁸ and —YR¹⁸; or when bonded to two contiguous atoms, together with the atoms to which they are attached, form a fused bicyclic or tricyclic heteroaryl, either of which may be optionally substituted.

14. The compound as recited in claim 13 wherein:
T is —C(O)OH, and tetrazolyl;
G¹ is —(CR¹R²)ₙ;
L¹ is selected from the group consisting of a bond, —S(O)₀₋₂(CH₂)₁₋₄O—, —O(CH₂)₁₋₄S(O)₀₋₂—, —CH₂S(O)₀₋₂—, —S(O)₀₋₂CH₂—, —S(O)₀₋₂—, —O(CH₂)₁₋₄O—, —CH₂O— and OCH₂—;
R¹⁴ is hydrogen;
R¹⁵ and R¹⁶ are independently selected from —R¹⁸ and —YR¹⁸; or R¹⁵ and R¹⁶ together with the atoms to which R¹⁵ and R¹⁶ are attached form 4,5-dihydro-naphtho[1,2-d]naphthal-2-yl, 4H-chromeno[4,3-d]naphthal-2-yl, 5,6-dihydro-4H-3-thia-1-aza-benzo[e]azulen-2-yl, benzthiazolyl, benzoxazolyl and 1-oxa-3-aza-cyclopenta[a]naphthalene-2-yl, any of which may be optionally substituted;
Y is selected from the group consisting of optionally substituted lower alkyl, optionally substituted lower alkenyl, —C(O)NH— and —O(CH₂)₁₋₃—; and
R¹⁸ is selected from the group consisting of —XOR⁴, —XC(O)OR⁴, —XC(O)OXOR⁴, —XC(O)N(R⁴)XOR⁵, —XC(O)N(R⁴)(R⁵), —XC(O)N(R⁴)XR⁵, phenyl, biphenyl, cyclohexyl, naphthyl, benzo[1,3]dioxol-5-yl, benzo[b]furanyl, pyridinyl, pyrimidinyl, dibenzo-furan-2-yl, furanyl, benzo[b]thiophene, thiophenyl, phenoxathiin-4-yl, benzoxazolyl, 3-oxo-3,4-dihydro-2H-benzo[1,4]oxazin-6-yl, 2-oxo-2,3-dihydro-benzooxazol-6-yl, 2,3-dihydro-benzo[1,4]dioxin-6-yl, benzoxazolyl, 3,4-dihydro-2H-benzo[b][1,4]-dioxepin-7-yl and quinolinyl; any of which may be optionally substituted.

15. The compound as recited in claim 14 having structural Formula (IIb) selected from the group consisting of:

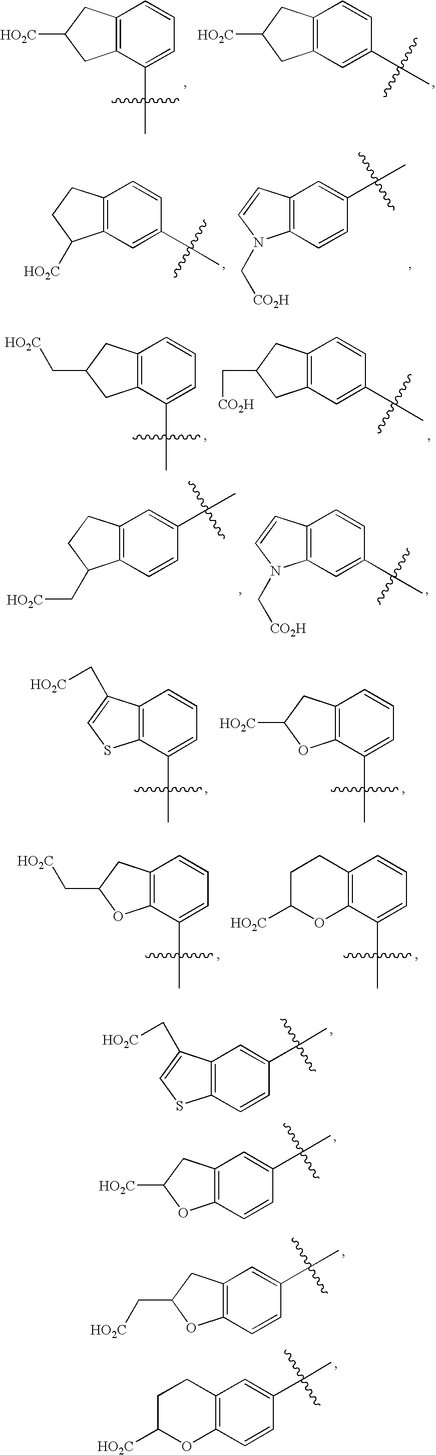

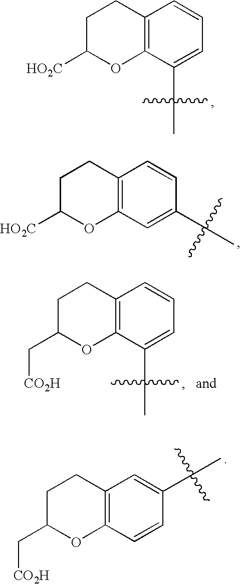

16. The compound as recited in claim 15 having the structural Formula VII: 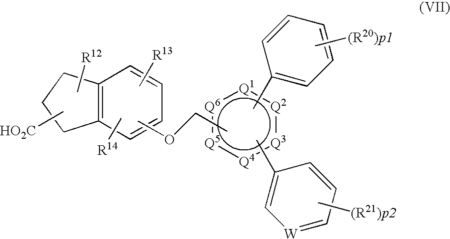
wherein:
W is selected from N and CH;
R¹², R¹³ and R¹⁴ are independently selected from the group consisting of hydrogen, optionally substituted lower alkyl, optionally substituted lower alkoxy and halogen;
R²⁰ is selected from trifluoromethyl and trifluoromethoxy;
R²¹ is selected from isopropyloxy and methoxy; and
p1 and p2 are independently selected from an integer from 0 to 2.

17. A compound as recited in claim 16 having the structural Formula selected from the group consisting of: 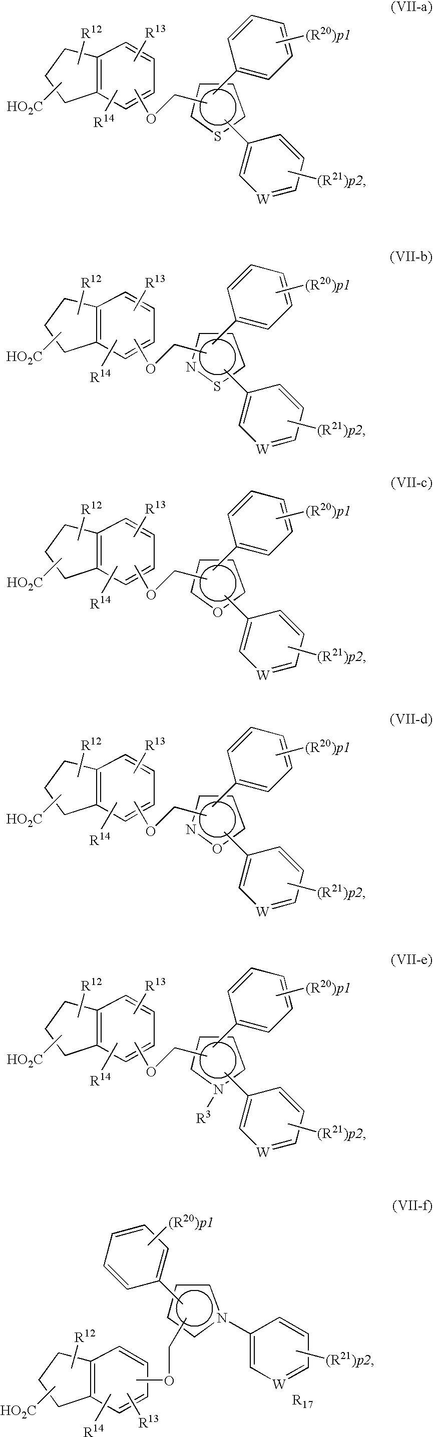 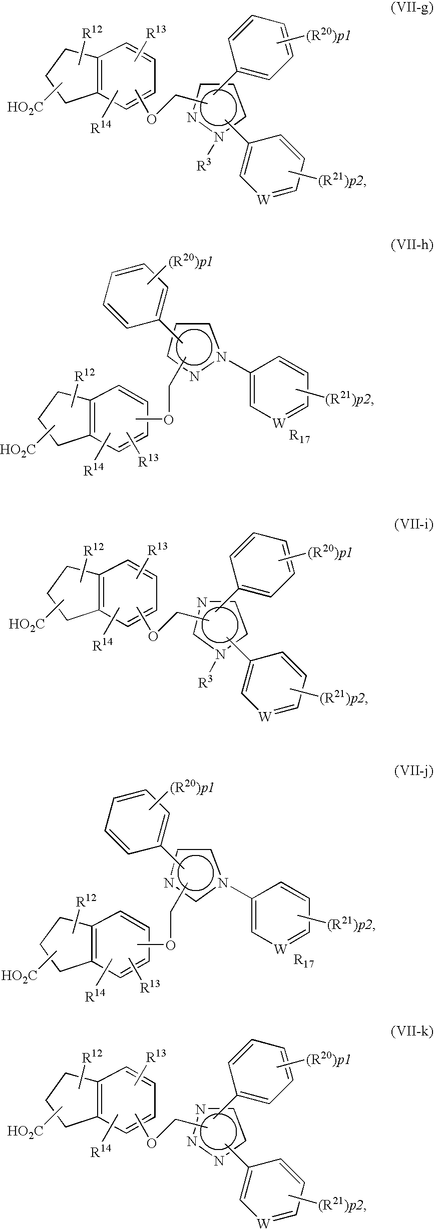 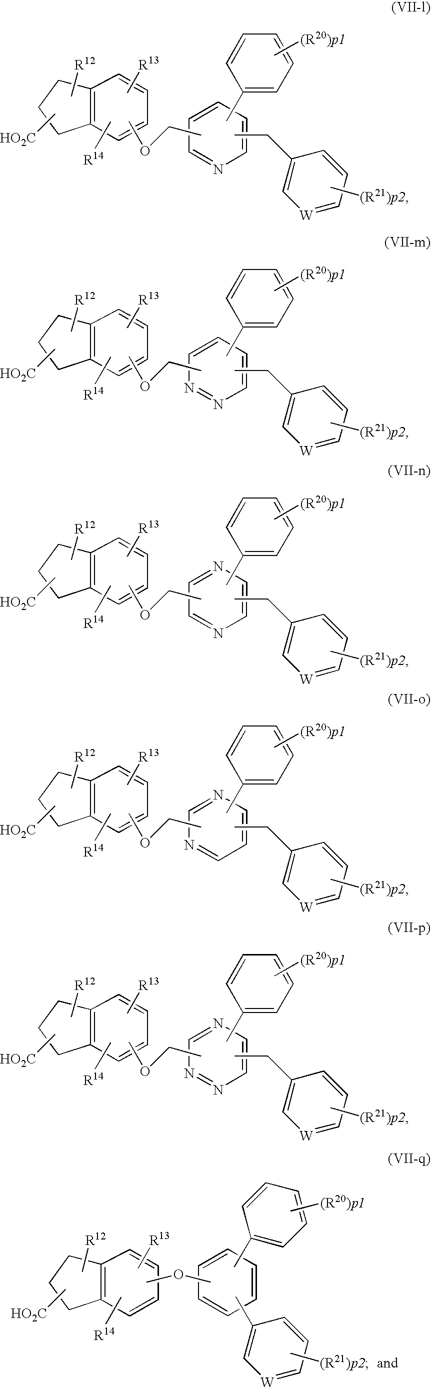
wherein the (R²⁰)ₚ₁-aryl and (R²¹)ₚ₂-(hetero)aryl moieties are attached to two contiguous central ring atoms.

18. The compound as recited in claim 15 having the structural Formula (VIII): 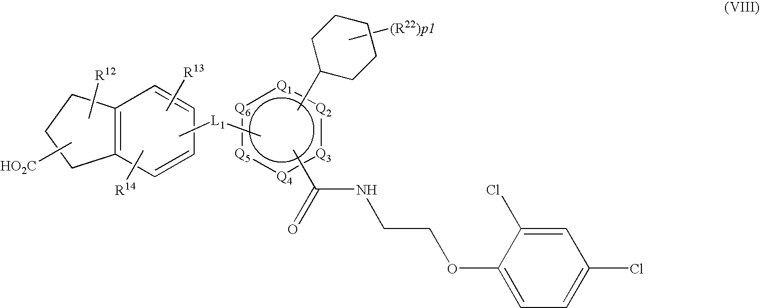
wherein:
L¹ from the group consisting of a bond, —CH₂O—, and —OCH₂—;
R¹², R¹³ and R¹⁴ are independently selected from the group consisting of hydrogen, lower alkyl, lower alkoxy and halogen;
R²² is selected from the group consisting of hydrogen, methoxy, isopropyloxy, trifluoromethoxy, fluoro, chloro, trifluoromethyl, methyl, and nitro; and
p1 is an integer from 0 to 2.

19. A compound as recited in claim 18 having the structural Formula selected from the group consisting of: 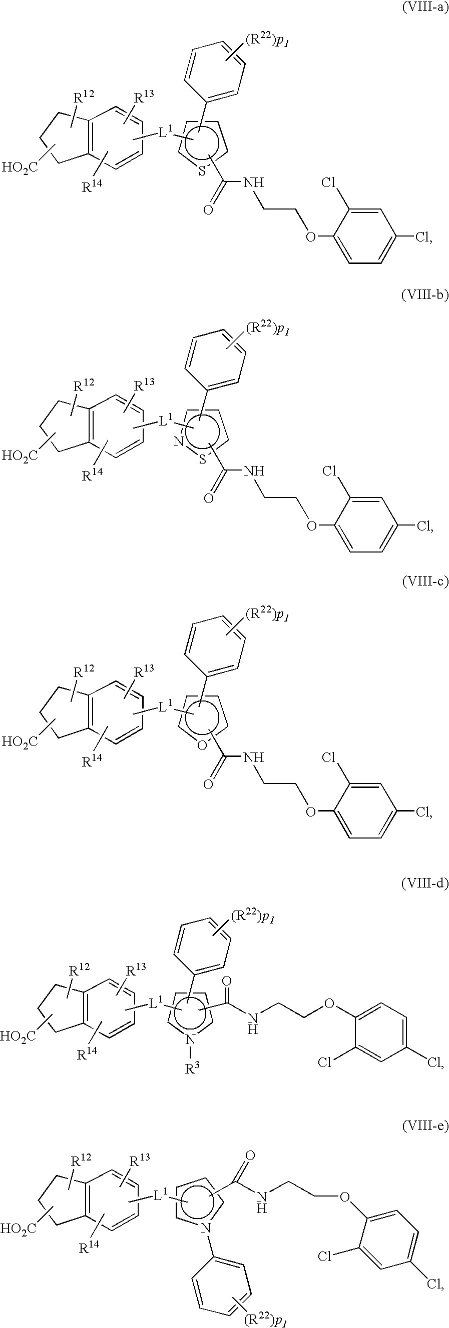 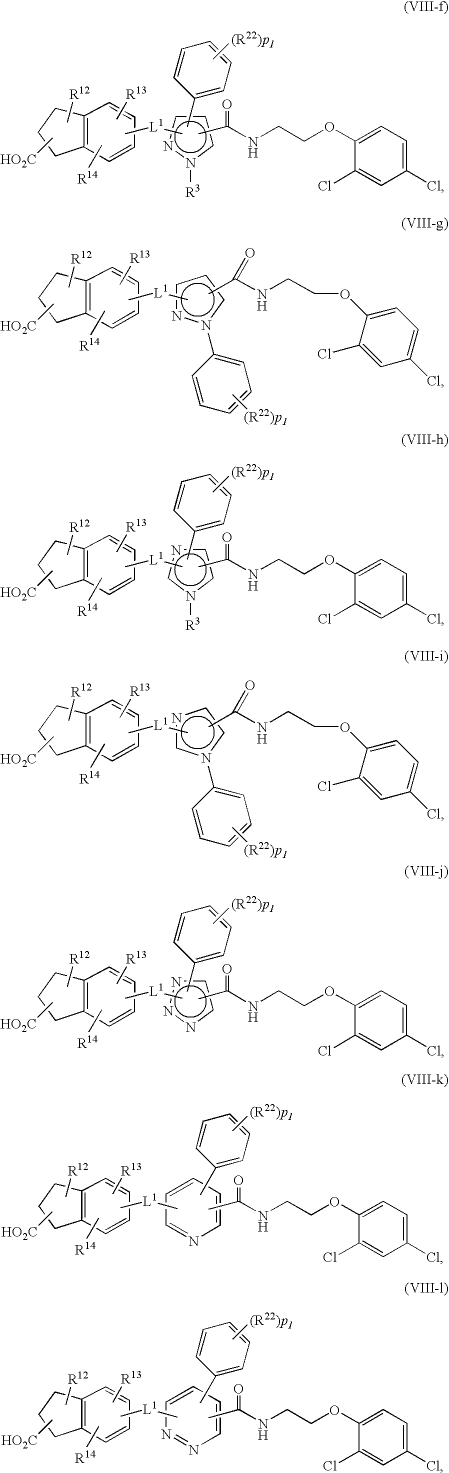 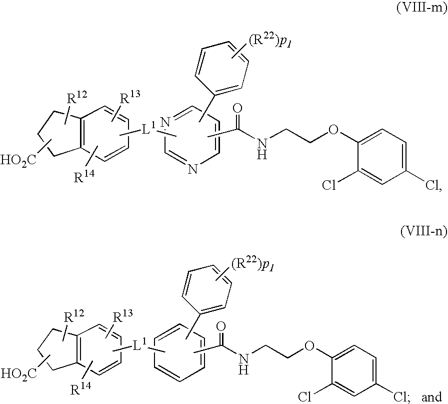
wherein the three substituents are attached to three contiguous ring atoms of the central ring moiety.

20. A method for treating a disease or disorder in an animal in which modulation of PPARδ activity can prevent, inhibit or ameliorate the pathology and/or symptomology of the disease, which method comprises administering to the animal a therapeutically effective amount of a compound of claim 1.

21. The method of claim 20 in which the disease or disorder is selected from the treatment of prophylaxis, dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, cachexia, inflammation, arthritis, cancer, anorexia, anorexia nervosa, bulimia, Alzheimer's disease, skin disorders, respiratory diseases, ophthalmic disorders, irritable bowel diseases, ulcerative colitis, Crohn's disease, type-1 diabetes, type-2 diabetes and Syndrome X.

22. The method of claim 20 in which the disease or disorder is selected from HIV wasting syndrome, long term critical illness, decreased muscle mass and/or muscle strength, decreased lean body mass, maintenance of muscle strength and function in the elderly, diminished muscle endurance and muscle function, and frailty in the elderly.

23. The use of a compound of claim 1 in the manufacture of a medicament for treating a disease in an animal in which PPARδ activity contributes to the pathology and/or symptomology of the disease.

24. A pharmaceutical composition comprising a therapeutically effective amount of a compound of claim 1 in combination with one or more pharmaceutically acceptable excipients. A pharmaceutical combination, especially a pharmaceutical composition, comprising: 1) a compound of claim 1 or a pharmaceutical acceptable salt thereof; and 2) at least one active ingredient selected from:
a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; dipeptidyl peptidase IV inhibitors such as DPP728, vildagliptin, MK-0431, saxagliptin, GSK23A; an AGE breaker; a thiazolidone derivative (glitazone) such as pioglitazone, rosiglitazone, or (R)-1-{4-[5-methyl-2-(4-trifluoromethylphenyl)-oxazol-4-ylmethoxy]-benzenesulfonyl}-2,3-dihydro-1H-indole-2-carboxylic acid, a non-glitazone type PPARy agonist e.g. GI-262570;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;
d) anti-hypertensive agents, e.g., loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; reninn inhibitors such as aliskiren, terlakiren, ditekiren, RO 66-1132, RO-66-1168; □-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amiodipine, bepridil, diltiazem, felodipine, nicardipine, nimodipine, nifedipine, nisoldipine and verapamil; aldosterone receptor antagonists; and aldosterone synthase inhibitors;
e) a HDL increasing compound;
f) a cholesterol absorption modulator such as Zetia® and KT6-971;
g) Apo-A1 analogues and mimetics;
h) thrombin inhibitors such as Ximelagatran;
i) aldosterone inhibitors such as anastrazole, fadrazole, epierenone;
j) inhibitors of platelet aggregation such as aspirin, clopidogrel bisulfate;
k) estrogen, testosterone, a selective estrogen receptor modulator, a selective androgen receptor modulator;
l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib or 4-Methyl-N-[3-(4-methyl-imidazol-1-yl)-5-trifluoromethylphenyl]-3-(4-pyridin-3-yl-pyrimidin-2-ylamino)-benzamide; and
m) an agent interacting with a 5-HT3 receptor and/or an agent interacting with 5-HT4 receptor such as tegaserod, tegaserod hydrogen maleate, cisapride, cilansetron; or, in each case, a pharmaceutically acceptable salt thereof; and optionally, together with a pharmaceutically acceptable carrier.
